SEQUENCE LISTING (138 sequences)

SEQ ID NO: 1 - protein, 16 aa, Artificial Sequence
  Description of Artificial Sequence Synthetic peptide
RSSQSLVHRNGNTYLH

SEQ ID NO: 2 - protein, 7 aa, Artificial Sequence
  Description of Artificial Sequence Synthetic peptide
TVSNRFS

SEQ ID NO: 3 - protein, 9 aa, Artificial Sequence
  Description of Artificial Sequence Synthetic peptide
SQSSHVPPT

SEQ ID NO: 4 - protein, 5 aa, Artificial Sequence
  Description of Artificial Sequence Synthetic peptide
NYGVN

SEQ ID NO: 5 - protein, 17 aa, Artificial Sequence
  Description of Artificial Sequence Synthetic peptide
WINPNTGEPTFDDDFKG

SEQ ID NO: 6 - protein, 11 aa, Artificial Sequence
  Description of Artificial Sequence Synthetic peptide
SRGKNEAWFAY

SEQ ID NO: 7 - protein, 10 aa, Artificial Sequence
  Description of Artificial Sequence Synthetic peptide
RASSSVSYIH

SEQ ID NO: 8 - protein, 7 aa, Artificial Sequence
  Description of Artificial Sequence Synthetic peptide
ATSNLAS

SEQ ID NO: 9 - protein, 9 aa, Artificial Sequence
  Description of Artificial Sequence Synthetic peptide
QQWTSNPPT

SEQ ID NO: 10 - protein, 5 aa, Artificial Sequence
  Description of Artificial Sequence Synthetic peptide
SYNMH

SEQ ID NO: 11 - protein, 16 aa, Artificial Sequence
  Description of Artificial Sequence Synthetic peptide
AIYPGNGTSYNQKFKG

SEQ ID NO: 12 - protein, 12 aa, Artificial Sequence
  Description of Artificial Sequence Synthetic peptide
STYYGGDWYFDV

SEQ ID NO: 13 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 16 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 17 - protein, 17 aa, Artificial Sequence
  Description of Artificial Sequence Synthetic peptide
QIEYLAKQIVDNAIQQA

SEQ ID NO: 18 - protein, 21 aa, Artificial Sequence
  Description of Artificial Sequence Synthetic peptide
CGQIEYLAKQIVDNAIQQAGC

SEQ ID NO: 19 - protein, 50 aa, Artificial Sequence
  Description of Artificial Sequence Synthetic polypeptide
SLRECELYVQKHNIQALLKDSIVQLCTARPERPMAFLREYFERLEKEEAK

SEQ ID NO: 20 - protein, 55 aa, Artificial Sequence
  Description of Artificial Sequence Synthetic polypeptide
MSCGGSLRECELYVQKHNIQALLKDSIVQLCTARPERPMAFLREYFERLEKEEAK

SEQ ID NO: 21 - protein, 23 aa, Artificial Sequence
  Description of Artificial Sequence Synthetic peptide
CGFEELAWKIAKMIWSDVFQQGC

SEQ ID NO: 22 - protein, 51 aa, Artificial Sequence
  Description of Artificial Sequence Synthetic polypeptide
SLRECELYVQKHNIQALLKDVSIVQLCTARPERPMAFLREYFEKLEKEEAK

SEQ ID NO: 23 - protein, 54 aa, Artificial Sequence
  Description of Artificial Sequence Synthetic polypeptide
SLKGCELYVQLHGIQQVLKDCIVHLCISKPERPMKFLREHFEKLEKEENRQILA

SEQ ID NO: 24 - protein, 44 aa, Artificial Sequence
  Description of Artificial Sequence Synthetic polypeptide
SHIQIPPGLTELLQGYTVEVGQQPPDLVDFAVEYFTRLREARRQ

SEQ ID NO: 25 - protein, 44 aa, Artificial Sequence
  Description of Artificial Sequence Synthetic polypeptide
SIEIPAGLTELLQGFTVEVLRHQPADLLEFALQHFTRLQQENER

SEQ ID NO: 26 - protein, 44 aa, Artificial Sequence
  Description of Artificial Sequence Synthetic consensus sequence
XXIXIPPXLXXLLXXYXVXVLXXXPPXLVXFXVXYFXXLXXXXX

SEQ ID NO: 27 - protein, 44 aa, Artificial Sequence
  Description of Artificial Sequence Synthetic polypeptide
THIQIPPGLTELLQGYTVEVLRQQPPDLVEFAVEYFTRLREARA

SEQ ID NO: 28 - protein, 44 aa, Artificial Sequence
  Description of Artificial Sequence Synthetic polypeptide
SKIQIPPGLTELLQGYTVEVLRQQPPDLVEFAVEYFTRLREARA

SEQ ID NO: 29 - protein, 44 aa, Artificial Sequence
  Description of Artificial Sequence Synthetic polypeptide
SRIQIPPGLTELLQGYTVEVLRQQPPDLVEFAVEYFTRLREARA

SEQ ID NO: 30 - protein, 44 aa, Artificial Sequence
  Description of Artificial Sequence Synthetic polypeptide
SHINIPPGLTELLQGYTVEVLRQQPPDLVEFAVEYFTRLREARA

SEQ ID NO: 31 - protein, 44 aa, Artificial Sequence
  Description of Artificial Sequence Synthetic polypeptide
SHIQIPPALTELLQGYTVEVLRQQPPDLVEFAVEYFTRLREARA

SEQ ID NO: 32 - protein, 44 aa, Artificial Sequence
  Description of Artificial Sequence Synthetic polypeptide
SHIQIPPGLSELLQGYTVEVLRQQPPDLVEFAVEYFTRLREARA

SEQ ID NO: 33 - protein, 44 aa, Artificial Sequence
  Description of Artificial Sequence Synthetic polypeptide
SHIQIPPGLTDLLQGYTVEVLRQQPPDLVEFAVEYFTRLREARA

SEQ ID NO: 34 - protein, 44 aa, Artificial Sequence
  Description of Artificial Sequence Synthetic polypeptide
SHIQIPPGLTELLNGYTVEVLRQQPPDLVEFAVEYFTRLREARA

SEQ ID NO: 35 - protein, 44 aa, Artificial Sequence
  Description of Artificial Sequence Synthetic polypeptide
SHIQIPPGLTELLQAYTVEVLRQQPPDLVEFAVEYFTRLREARA

SEQ ID NO: 36 - protein, 44 aa, Artificial Sequence
  Description of Artificial Sequence Synthetic polypeptide
SHIQIPPGLTELLQGYSVEVLRQQPPDLVEFAVEYFTRLREARA

SEQ ID NO: 37 - protein, 44 aa, Artificial Sequence
  Description of Artificial Sequence Synthetic polypeptide
SHIQIPPGLTELLQGYTVDVLRQQPPDLVEFAVEYFTRLREARA

SEQ ID NO: 38 - protein, 44 aa, Artificial Sequence
  Description of Artificial Sequence Synthetic polypeptide
SHIQIPPGLTELLQGYTVEVLKQQPPDLVEFAVEYFTRLREARA

SEQ ID NO: 39 - protein, 44 aa, Artificial Sequence
  Description of Artificial Sequence Synthetic polypeptide
SHIQIPPGLTELLQGYTVEVLRNQPPDLVEFAVEYFTRLREARA

SEQ ID NO: 40 - protein, 44 aa, Artificial Sequence
  Description of Artificial Sequence Synthetic polypeptide
SHIQIPPGLTELLQGYTVEVLRQNPPDLVEFAVEYFTRLREARA

SEQ ID NO: 41 - protein, 44 aa, Artificial Sequence
  Description of Artificial Sequence Synthetic polypeptide
SHIQIPPGLTELLQGYTVEVLRQQPPELVEFAVEYFTRLREARA

SEQ ID NO: 42 - protein, 44 aa, Artificial Sequence
  Description of Artificial Sequence Synthetic polypeptide
SHIQIPPGLTELLQGYTVEVLRQQPPDLVDFAVEYFTRLREARA

SEQ ID NO: 43 - protein, 44 aa, Artificial Sequence
  Description of Artificial Sequence Synthetic polypeptide
SHIQIPPGLTELLQGYTVEVLRQQPPDLVEFLVEYFTRLREARA

SEQ ID NO: 44 - protein, 44 aa, Artificial Sequence
  Description of Artificial Sequence Synthetic polypeptide
SHIQIPPGLTELLQGYTVEVLRQQPPDLVEFIVEYFTRLREARA

SEQ ID NO: 45 - protein, 44 aa, Artificial Sequence
  Description of Artificial Sequence Synthetic polypeptide
SHIQIPPGLTELLQGYTVEVLRQQPPDLVEFVVEYFTRLREARA

SEQ ID NO: 46 - protein, 44 aa, Artificial Sequence
  Description of Artificial Sequence Synthetic polypeptide
SHIQIPPGLTELLQGYTVEVLRQQPPDLVEFAVDYFTRLREARA

SEQ ID NO: 47 - protein, 17 aa, Artificial Sequence
  Description of Artificial Sequence Synthetic peptide
NIEYLAKQIVDNAIQQA

SEQ ID NO: 48 - protein, 17 aa, Artificial Sequence
  Description of Artificial Sequence Synthetic peptide
QLEYLAKQIVDNAIQQA

SEQ ID NO: 49 - protein, 17 aa, Artificial Sequence
  Description of Artificial Sequence Synthetic peptide
QVEYLAKQIVDNAIQQA

SEQ ID NO: 50 - protein, 17 aa, Artificial Sequence
  Description of Artificial Sequence Synthetic peptide
QIDYLAKQIVDNAIQQA

SEQ ID NO: 51 - protein, 17 aa, Artificial Sequence
  Description of Artificial Sequence Synthetic peptide
QIEFLAKQIVDNAIQQA

SEQ ID NO: 52 - protein, 17 aa, Artificial Sequence
  Description of Artificial Sequence Synthetic peptide
QIETLAKQIVDNAIQQA

SEQ ID NO: 53 - protein, 17 aa, Artificial Sequence
  Description of Artificial Sequence Synthetic peptide
QIESLAKQIVDNAIQQA

SEQ ID NO: 54 - protein, 17 aa, Artificial Sequence
  Description of Artificial Sequence Synthetic peptide
QIEYIAKQIVDNAIQQA

SEQ ID NO: 55 - protein, 17 aa, Artificial Sequence
  Description of Artificial Sequence Synthetic peptide
QIEYVAKQIVDNAIQQA

SEQ ID NO: 56 - protein, 17 aa, Artificial Sequence
  Description of Artificial Sequence Synthetic peptide
QIEYLARQIVDNAIQQA

SEQ ID NO: 57 - protein, 17 aa, Artificial Sequence
  Description of Artificial Sequence Synthetic peptide
QIEYLAKNIVDNAIQQA

SEQ ID NO: 58 - protein, 17 aa, Artificial Sequence
  Description of Artificial Sequence Synthetic peptide
QIEYLAKQIVENAIQQA

SEQ ID NO: 59 - protein, 17 aa, Artificial Sequence
  Description of Artificial Sequence Synthetic peptide
QIEYLAKQIVDQAIQQA

SEQ ID NO: 60 - protein, 17 aa, Artificial Sequence
  Description of Artificial Sequence Synthetic peptide
QIEYLAKQIVDNAINQA

SEQ ID NO: 61 - protein, 17 aa, Artificial Sequence
  Description of Artificial Sequence Synthetic peptide
QIEYLAKQIVDNAIQNA

SEQ ID NO: 62 - protein, 17 aa, Artificial Sequence
  Description of Artificial Sequence Synthetic peptide
QIEYLAKQIVDNAIQQL

SEQ ID NO: 63 - protein, 17 aa, Artificial Sequence
  Description of Artificial Sequence Synthetic peptide
QIEYLAKQIVDNAIQQI

SEQ ID NO: 64 - protein, 17 aa, Artificial Sequence
  Description of Artificial Sequence Synthetic peptide
QIEYLAKQIVDNAIQQV

SEQ ID NO: 65 - protein, 17 aa, Artificial Sequence
  Description of Artificial Sequence Synthetic consensus sequence
XXXXXAXXIVXXAIXXX

SEQ ID NO: 66 - protein, 17 aa, Artificial Sequence
  Description of Artificial Sequence Synthetic peptide
QIEYVAKQIVDYAIHQA

SEQ ID NO: 67 - protein, 17 aa, Artificial Sequence
  Description of Artificial Sequence Synthetic peptide
QIEYKAKQIVDHAIHQA

SEQ ID NO: 68 - protein, 17 aa, Artificial Sequence
  Description of Artificial Sequence Synthetic peptide
QIEYHAKQIVDHAIHQA

SEQ ID NO: 69 - protein, 17 aa, Artificial Sequence
  Description of Artificial Sequence Synthetic peptide
QIEYVAKQIVDHAIHQA

SEQ ID NO: 70 - protein, 18 aa, Artificial Sequence
  Description of Artificial Sequence Synthetic peptide
PLEYQAGLLVQNAIQQAI

SEQ ID NO: 71 - protein, 18 aa, Artificial Sequence
  Description of Artificial Sequence Synthetic peptide
LLIETASSLVKNAIQLSI

SEQ ID NO: 72 - protein, 18 aa, Artificial Sequence
  Description of Artificial Sequence Synthetic peptide
LIEEAASRIVDAVIEQVK

SEQ ID NO: 73 - protein, 18 aa, Artificial Sequence
  Description of Artificial Sequence Synthetic peptide
ALYQFADRFSELVISEAL

SEQ ID NO: 74 - protein, 17 aa, Artificial Sequence
  Description of Artificial Sequence Synthetic peptide
LEQVANQLADQIIKEAT

SEQ ID NO: 75 - protein, 17 aa, Artificial Sequence
  Description of Artificial Sequence Synthetic peptide
FEELAWKIAKMIWSDVF

SEQ ID NO: 76 - protein, 18 aa, Artificial Sequence
  Description of Artificial Sequence Synthetic peptide
ELVRLSKRLVENAVLKAV

SEQ ID NO: 77 - protein, 18 aa, Artificial Sequence
  Description of Artificial Sequence Synthetic peptide
TAEEVSARIVQVVTAEAV

SEQ ID NO: 78 - protein, 18 aa, Artificial Sequence
  Description of Artificial Sequence Synthetic peptide
QIKQAAFQLISQVILEAT

SEQ ID NO: 79 - protein, 16 aa, Artificial Sequence
  Description of Artificial Sequence Synthetic peptide
LAWKIAKMIVSDVMQQ

SEQ ID NO: 80 - protein, 24 aa, Artificial Sequence
  Description of Artificial Sequence Synthetic peptide
DLIEEAASRIVDAVIEQVKAAGAY

SEQ ID NO: 81 - protein, 18 aa, Artificial Sequence
  Description of Artificial Sequence Synthetic peptide
LEQYANQLADQIIKEATE

SEQ ID NO: 82 - protein, 20 aa, Artificial Sequence
  Description of Artificial Sequence Synthetic peptide
FEELAWKIAKMIWSDVFQQC

SEQ ID NO: 83 - protein, 17 aa, Artificial Sequence
  Description of Artificial Sequence Synthetic peptide
QIEYLAKQIPDNAIQQA

SEQ ID NO: 84 - protein, 25 aa, Artificial Sequence
  Description of Artificial Sequence Synthetic peptide
KGADLIEEAASRIVDAVIEQVKAAG

SEQ ID NO: 85 - protein, 25 aa, Artificial Sequence
  Description of Artificial Sequence Synthetic peptide
KGADLIEEAASRIPDAPIEQVKAAG

SEQ ID NO: 86 - protein, 25 aa, Artificial Sequence
  Description of Artificial Sequence Synthetic peptide
PEDAELVRLSKRLVENAVLKAVQQY

SEQ ID NO: 87 - protein, 25 aa, Artificial Sequence
  Description of Artificial Sequence Synthetic peptide
PEDAELVRTSKRLVENAVLKAVQQY

SEQ ID NO: 88 - protein, 25 aa, Artificial Sequence
  Description of Artificial Sequence Synthetic peptide
PEDAELVRLSKRDVENAVLKAVQQY

SEQ ID NO: 89 - protein, 25 aa, Artificial Sequence
  Description of Artificial Sequence Synthetic peptide
PEDAELVRLSKRLPENAVLKAVQQY

SEQ ID NO: 90 - protein, 25 aa, Artificial Sequence
  Description of Artificial Sequence Synthetic peptide
PEDAELVRLSKRLPENAPLKAVQQY

SEQ ID NO: 91 - protein, 25 aa, Artificial Sequence
  Description of Artificial Sequence Synthetic peptide
PEDAELVRLSKRLVENAVEKAVQQY

SEQ ID NO: 92 - protein, 25 aa, Artificial Sequence
  Description of Artificial Sequence Synthetic peptide
EEGLDRNEEIKRAAFQIISQVISEA

SEQ ID NO: 93 - protein, 25 aa, Artificial Sequence
  Description of Artificial Sequence Synthetic peptide
LVDDPLEYQAGLLVQNAIQQAIAEQ

SEQ ID NO: 94 - protein, 25 aa, Artificial Sequence
  Description of Artificial Sequence Synthetic peptide
QYETLLIETASSLVKNAIQLSIEQL

SEQ ID NO: 95 - protein, 25 aa, Artificial Sequence
  Description of Artificial Sequence Synthetic peptide
LEKQYQEQLEEEVAKVIVSMSIAFA

SEQ ID NO: 96 - protein, 25 aa, Artificial Sequence
  Description of Artificial Sequence Synthetic peptide
NTDEAQEELAWKIAKMIVSDIMQQA

SEQ ID NO: 97 - protein, 25 aa, Artificial Sequence
  Description of Artificial Sequence Synthetic peptide
VNLDKKAVLAEKIVAEAIEKAEREL

SEQ ID NO: 98 - protein, 25 aa, Artificial Sequence
  Description of Artificial Sequence Synthetic peptide
NGILELETKSSKLVQNIIQTAVDQF

SEQ ID NO: 99 - protein, 24 aa, Artificial Sequence
  Description of Artificial Sequence Synthetic peptide
QDKNYEDELTQVALALVEDVINYA

SEQ ID NO: 100 - protein, 25 aa, Artificial Sequence
  Description of Artificial Sequence Synthetic peptide
ETSAKDNINIEEAARFLVEKILVNH

SEQ ID NO: 101 - protein, 44 aa, Artificial Sequence
  Description of Artificial Sequence Synthetic consensus sequence
XHIXIPPGLXELLQGYTXEVLRXQPPDLVEFAXXYFXXLXEXRX

SEQ ID NO: 102 - protein, 16 aa, Artificial Sequence
  Description of Artificial Sequence Synthetic peptide
EFPKPSTPPGSSGGAP

SEQ ID NO: 103 - protein, 4 aa, Artificial Sequence
  Description of Artificial Sequence Synthetic peptide
FKYK

SEQ ID NO: 104 - DNA, 21 bp, Artificial Sequence
  Description of Artificial Sequence Synthetic oligonucleotide
aatgcggcggtggtgacagta

SEQ ID NO: 105 - DNA, 21 bp, Artificial Sequence
  Description of Artificial Sequence Synthetic oligonucleotide
aagctcagcacacagaaagac

SEQ ID NO: 106 - DNA, 21 bp, Artificial Sequence
  Description of Artificial Sequence Synthetic oligonucleotide
uaaaaucuuccugcccacctt

SEQ ID NO: 107 - DNA, 21 bp, Artificial Sequence
  Description of Artificial Sequence Synthetic oligonucleotide
ggaagcuguuggcugaaaatt

SEQ ID NO: 108 - RNA, 21 nt, Artificial Sequence
  Description of Artificial Sequence Synthetic oligonucleotide
aagaccagccucuuugcccag

SEQ ID NO: 109 - DNA, 19 bp, Artificial Sequence
  Description of Artificial Sequence Synthetic oligonucleotide
ggaccaggcagaaaacgag

SEQ ID NO: 110 - RNA, 17 nt, Artificial Sequence
  Description of Artificial Sequence Synthetic oligonucleotide
cuaucaggaugacgcgg

SEQ ID NO: 111 - RNA, 21 nt, Artificial Sequence
  Description of Artificial Sequence Synthetic oligonucleotide
ugacacaggcaggcuugacuu

SEQ ID NO: 112 - DNA, 19 bp, Artificial Sequence
  Description of Artificial Sequence Synthetic oligonucleotide
ggtgaagaagggcgtccaa

SEQ ID NO: 113 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 114 - DNA, 20 bp, Artificial Sequence
  Description of Artificial Sequence Synthetic oligonucleotide
aggtggtgttaacagcagag

SEQ ID NO: 115 - DNA, 21 bp, Artificial Sequence
  Description of Artificial Sequence Synthetic oligonucleotide
aaggtggagcaagcggtggag

SEQ ID NO: 116 - DNA, 21 bp, Artificial Sequence
  Description of Artificial Sequence Synthetic oligonucleotide
aaggagttgaaggccgacaaa

SEQ ID NO: 117 - DNA, 21 bp, Artificial Sequence
  Description of Artificial Sequence Synthetic oligonucleotide
uauggagcugcagaggaugtt

SEQ ID NO: 118 - DNA, 49 bp, Artificial Sequence
  Description of Artificial Sequence Synthetic oligonucleotide
tttgaatatctgtgctgagaacacagttctcagcacagatattcttttt

SEQ ID NO: 119 - DNA, 29 bp, Artificial Sequence
  Description of Artificial Sequence Synthetic oligonucleotide
aatgagaaaagcaaaaggtgccctgtctc

SEQ ID NO: 120 - RNA, 21 nt, Artificial Sequence
  Description of Artificial Sequence Synthetic oligonucleotide
aaucaucaucaagaaagggca

SEQ ID NO: 121 - DNA, 21 bp, Artificial Sequence
  Description of Artificial Sequence Synthetic oligonucleotide
augacugucaggauguugctt

SEQ ID NO: 122 - RNA, 21 nt, Artificial Sequence
  Description of Artificial Sequence Synthetic oligonucleotide
gaacgaauccugaagacaucu

SEQ ID NO: 123 - DNA, 29 bp, Artificial Sequence
  Description of Artificial Sequence Synthetic oligonucleotide
aagcctggctacagcaatatgcctgtctc

SEQ ID NO: 124 - DNA, 21 bp, Artificial Sequence
  Description of Artificial Sequence Synthetic oligonucleotide
ugaccaucaccgaguuuautt

SEQ ID NO: 125 - DNA, 21 bp, Artificial Sequence
  Description of Artificial Sequence Synthetic oligonucleotide
aagtcggacgcaacagagaaa

SEQ ID NO: 126 - DNA, 21 bp, Artificial Sequence
  Description of Artificial Sequence Synthetic oligonucleotide
cuaccuuucuacggacgugtt

SEQ ID NO: 127 - DNA, 21 bp, Artificial Sequence
  Description of Artificial Sequence Synthetic oligonucleotide
ctgcctaaggcggatttgaat

SEQ ID NO: 128 - DNA, 21 bp, Artificial Sequence
  Description of Artificial Sequence Synthetic oligonucleotide
ttauuccuucuucgggaaguc

SEQ ID NO: 129 - DNA, 21 bp, Artificial Sequence
  Description of Artificial Sequence Synthetic oligonucleotide
aaccttctggaacccgcccac

SEQ ID NO: 130 - DNA, 19 bp, Artificial Sequence
  Description of Artificial Sequence Synthetic oligonucleotide
gagcatcttcgagcaagaa

SEQ ID NO: 131 - DNA, 19 bp, Artificial Sequence
  Description of Artificial Sequence Synthetic oligonucleotide
catgtggcaccgtttgcct

SEQ ID NO: 132 - DNA, 21 bp, Artificial Sequence
  Description of Artificial Sequence Synthetic oligonucleotide
aactaccagaaaggtatacct

SEQ ID NO: 133 - DNA, 21 bp, Artificial Sequence
  Description of Artificial Sequence Synthetic oligonucleotide
ucacaguguccuuuauguatt

SEQ ID NO: 134 - DNA, 21 bp, Artificial Sequence
  Description of Artificial Sequence Synthetic oligonucleotide
gcaugaaccggaggcccautt

SEQ ID NO: 135 - DNA, 19 bp, Artificial Sequence
  Description of Artificial Sequence Synthetic oligonucleotide
ccggacagttccatgtata

SEQ ID NO: 136 - protein, 11 aa, Artificial Sequence
  Description of Artificial Sequence Synthetic peptide
ACSSSPSKHCG

SEQ ID NO: 137 - protein, 8 aa, Artificial Sequence
  Description of Artificial Sequence Synthetic peptide
FCIGRLCG

SEQ ID NO: 138 - protein, 12 aa, Human immunodeficiency virus
GRKKRRNRRRCG

(19) United States
(12) Patent Application Publication
(10) Pub. No.: US 2016/0120976 A1
(43) Pub. Date: May 5, 2016

(54) COMBINATION THERAPY WITH ANTI-CD74 AND ANTI-CD20 ANTIBODIES IN PATIENTS WITH RELAPSED AND REFRACTORY B-CELL NON-HODGKIN'S LYMPHOMA

(71) Applicant: Immunomedics, Inc., Morris Plains, NJ (US)

(72) Inventors: David M. Goldenberg, Mendham, NJ (US); William A. Wegener, Broomall, PA (US)

(21) Appl. No.: 14/992,528

(22) Filed: Jan. 11, 2016

Related U.S. Application Data
(63) Continuation-in-part of application No. 14/520,596, filed on Oct. 22, 2014.
Application No. 14/520,596 is a division of application No. 13/904,534, filed on May 29, 2013, now Pat. No. 8,906,378.
Application No. 13/904,534 is a division of application No. 13/209,954, filed on Aug. 15, 2011, now Pat. No. 8,475,794.
(60) Provisional application No. 62/106,995, filed on Jan. 23, 2015.
(60) Provisional application No. 61/374,751, filed on Aug. 18, 2010.
(60) Provisional application No. 61/374,772, filed on Aug. 18, 2010.
(60) Provisional application No. 61/508,871, filed on Jul. 18, 2011.

(51) Int. Cl. A61K 39/395; A61K 31/425; A61K 31/167; A61K 31/135; A61K 31/573; A61K 31/133

(52) U.S. Cl. CPC A61K 39/3955; A61K 31/573; A61K 31/133; A61K 31/167; A61K 31/135; A61K 31/425; A61K 2039/545

(57) ABSTRACT

The present invention concerns methods of treating relapsed/resistant non-Hodgkin's lymphoma using combination therapy with an anti-CD20 antibody or fragment and an anti-CD74 antibody or fragment. In preferred embodiments, the antibody combination is administered along with at least one other therapeutic agent. The combination is effective to treat indolent NHL that is resistant to or relapsed from at least one therapy for NHL, including but not limited to rituximab resistant NHL. The antibody combination may be administered to human subjects at specific dosages and dosing schedules, that are effective to treat the disease but do not induce a dose-limiting toxicity.

### CROSS-REFERENCE TO RELATED APPLICATIONS

This application claims the benefit under 35 U.S.C. 119(e) of provisional U.S. Patent Application Ser. No. 62/106,995, filed Jan. 23, 2015, which is incorporated herein by reference in its entirety. This application is a continuation-in-part of U.S. patent application Ser. No. 14/520,596, filed Oct. 22, 2014, which was a divisional of U.S. patent application Ser. No. 13/904,534 (now U.S. Pat. No. 8,906,378), filed May 29, 2013, which was a divisional of U.S. patent application Ser. No. 13/209,954 (now U.S. Pat. No. 8,475,794), filed Aug. 15, 2011, which claimed the benefit under 35 U.S.C. 119(e) of provisional U.S. Patent Application Ser. No. 61/374,751, filed Aug. 18, 2010; 61/374,772, filed Aug. 18, 2010, and 61/508,871, filed Jul. 18, 2011.

### SEQUENCE LISTING

The instant application contains a Sequence Listing which has been submitted in ASCII format via EFS-Web and is hereby incorporated by reference in its entirety. Said ASCII copy, created on Jan. 5, 2016, is named IMM355US1_SL.txt and is 56,385 bytes in size.

### FIELD OF THE INVENTION

The present invention concerns compositions and methods of use of combination therapy using at least one anti-CD74 antibody or antigen-binding fragment thereof and at least one anti-CD20 antibody or antigen-binding fragment thereof. The compositions and methods are not limiting and the anti-CD74 and anti-CD20 antibodies or fragments may be utilized in combination with one or more additional therapeutic agents. In preferred embodiments, the other therapeutic agent may be another antibody or fragment thereof against the same or a different target antigen, including but not limited to CD 19, CD20, CD21, CD22, CD23, CD37, CD40, CD40L, CD52, CD80 or HLA-DR. In other embodiments, the other therapeutic agent may be an immunomodulator, a cytotoxic agent, a drug, a toxin, an anti-angiogenic agent, a proapoptotic agent or a radionuclide. The compositions and methods are of use to treat human patients with relapsed/refractory B-cell non-Hodgkin's lymphoma (NHL), including but not limited to follicular lymphoma, diffuse large B-cell lymphoma (DLBCL), mantle cell lymphoma, lymphoplasmacytic lymphoma and marginal zone lymphoma. Preferably, the patients to be treated have an indolent form of NHL. The combination of anti-CD74 and anti-CD20 antibodies or fragments thereof is effective to treat patients who are refractory to or relapsed from previous treatment with standard therapies against NHL. Preferably, the patients to be treated are refractory to and/or relapsed from rituximab therapy. More preferably, the patients may be treated with an induction dose of anti-CD20 antibody at 200 mg/m² per week, with anti-CD74 antibody administered at a dose of 8, 16 or 20 mg/kg once or twice a week. In particularly preferred embodiments, the anti-CD20 antibody is veltuzumab (hA20) and the anti-CD74 antibody is milatuzumab (hLL1). In most preferred embodiments, the combination of anti-CD74 and anti-CD20 antibodies or fragments thereof is significantly more efficacious for treating indolent NHL than either agent administered alone or the sum of effects of the two agents administered separately.

### BACKGROUND

To address the clinical concerns of immunogenicity and suboptimal pharmacokinetics, cancer therapy with monoclonal antibodies has evolved from murine to chimeric, humanized, and fully human constructs. Parallel to these improvements have been continuing efforts to develop more effective forms of antibodies, which to date include different antibody isotypes, single-chain antibody fragments with monomeric or multimeric binding moieties, specific mutations in the Fc region to modulate effector function or circulating half-life, and bispecific antibodies of numerous designs that vary in valency, structure, and constituents (Chames et al., Br J Pharmacol 2009, 157:220-233).

Because signaling pathway redundancies can result in lack of response to a single antibody, diverse strategies to use combination therapy with antibodies that bind to different epitopes or different antigens on the same target cell have been proposed. Combinations such as anti-CD20 and anti-CD22 (Stein et al., Clin Cancer Res 2004, 10:2868-2878), anti-CD20 and anti-HLA-DR (Tobin et al., Leuk Lymphoma 2007, 48:944-956), anti-CD20 and anti-TRAIL-R1 (Maddipatla et al., Clin Cancer Res 2007, 13:4556-4564), anti-IGF-1R and anti-EGFR (Goetsche et al., Int J Cancer 2005, 113:316-328), anti-IGF-1R and anti-VEGF (Shang et al., Mol Cancer Ther 2008, 7:2599-2608), or trastuzumab and pertuzumab that target different regions of human EGFR2 (Nahta et al., Cancer Res 2004, 64:2343-2346) have been evaluated preclinically, showing enhanced or synergistic antitumor activity in vitro and in vivo.

The first clinical evidence of an apparent advantage of combining two antibodies against different cancer cell antigens involved the administration of rituximab (chimeric anti-CD20) and epratuzumab (humanized anti-CD22 antibody) in patients with non-Hodgkin lymphoma (NHL). The combination was found to enhance anti-lymphoma efficacy without a commensurate increase in toxicity, based on 3 independent clinical trials (Leonard et al., J Clin Oncol 2005, 23:5044-5051). Although these results are promising, a need exists in the field for more effective antibody-based combination therapies.

### SUMMARY

The present invention concerns improved compositions and methods of use of combination therapy with at least one anti-CD74 and at least one anti-CD20 antibody or fragment thereof. The combination may be used alone, or else with one or more other therapeutic agents. The combination therapy is of use to treat human patients with relapsed/refractory B-cell non-Hodgkin's lymphoma. Surprisingly, the combination is effective to treat patients who had previously relapsed from or shown resistance to standard therapies for NHL, such as radiation therapy, rituximab, CHOP or R-CHOP.

Many examples of anti-CD74 antibodies are known in the art and any such known antibody or fragment thereof may be utilized. In a preferred embodiment, the anti-CD74 antibody is an hLL1 antibody (also known as milatuzumab) that comprises the light chain complementarity-determining region (CDR) sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). A humanized LL1 (hLL1) anti-CD74 antibody suitable for use is disclosed in U.S. Pat. No. 7,312,318, incorporated herein by reference from Col. 35, line 1 through Col. 42, line 27 and FIG. 1 through FIG. 4. However, in alternative embodiments, other known and/or commercially available anti-CD74 antibodies may be utilized, such as LS-B1963, LS-B2594, LS-B1859, LS-B2598, LS-05525, LS-C44929, etc. (LSBio, Seattle, Wash.); LN2 (BIOLEGEND®, San Diego, Calif.); PIN.1, SPM523, LN3, CerCLIP.1 (ABCAM®, Cambridge, Mass.); At14/19, Bu45 (SEROTEC®, Raleigh, N.C.); 1D1 (ABNOVA®, Taipei City, Taiwan); 5-329 (EBIOSCIENCE®, San Diego, Calif.); and any other antagonistic anti-CD74 antibody known in the art.

The anti-CD74 antibody may be selected such that it competes with or blocks binding to CD74 of an LL1 antibody comprising the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). Alternatively, the anti-CD74 antibody may bind to the same epitope of CD74 as an LL1 antibody.

Many examples of anti-CD20 antibodies are known in the art and any such known antibody or fragment thereof may be utilized. In a preferred embodiment, the anti-CD20 antibody is an hA20 antibody (also known as veltuzumab) that comprises the light chain complementarity-determining region (CDR) sequences CDR1 (RASSSVSYIH; SEQ ID NO:7), CDR2 (ATSNLAS; SEQ ID NO:8), and CDR3 (QQWTSNPPT; SEQ ID NO:9) and the heavy chain variable region CDR sequences CDR1 (SYNMH; SEQ ID NO:10), CDR2 (AIYPGNGDTSYNQKFKG; SEQ ID NO:11), and CDR3 (STYYGGDWYFDV; SEQ ID NO:12).

A humanized anti-CD20 antibody suitable for use is disclosed in U.S. Pat. No. 7,435,803, incorporated herein by reference from Col. 36, line 4 through Col. 46, line 52 and FIGS. 1, 2, 4, 5 and 7. However, in alternative embodiments, other known and/or commercially available anti-CD20 antibodies may be utilized, such as rituximab; ofatumumab; ibritumomab; tositumomab; ocrelizumab; GA101; BCX-301; DXL 625; L26, B-Lyl, MEM-97, LT20, 2H7, AT80, B-H20 (ABCAM®, Cambridge, Mass.); HI20a, HI47, 13.6E12 (ABBIOTEC®, San Diego, Calif.); 4f11, 5c11, 7d1 (ABD SEROTEC®, Raleigh, N.C.) and any other anti-CD20 antibody known in the art.

The anti-CD20 antibody may be selected such that it competes with or blocks binding to CD20 of an hA20 antibody comprising the light chain complementarity-determining region (CDR) sequences CDR1 (RASSSVSYIH; SEQ ID NO:7), CDR2 (ATSNLAS; SEQ ID NO:8), and CDR3 (QQWTSNPPT; SEQ ID NO:9) and the heavy chain variable region CDR sequences CDR1 (SYNMH; SEQ ID NO:10), CDR2 (AIYPGNGDTSYNQKFKG; SEQ ID NO:11), and CDR3 (STYYGGDWYFDV; SEQ ID NO:12). Alternatively, the anti-CD20 antibody may bind to the same epitope of CD20 as a hA20 antibody.

The antibodies or fragments thereof may be used as naked antibodies, alone or in combination with one or more therapeutic agents. Alternatively, the antibodies or fragments may be utilized as immunoconjugates, attached to one or more therapeutic agents. (For methods of making immunoconjugates, see, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.) Therapeutic agents may be selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide molecule (e.g., an antisense molecule or a gene) or another antibody or fragment thereof.

The therapeutic agent may be selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

The therapeutic agent may comprise a radionuclide selected from the group consisting of ^{103m}Rh, ¹⁹³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³H, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo, ^{99m}Tc and ²²⁷Th.

The therapeutic agent may be an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

An immunomodulator of use may be selected from the group consisting of a cytokine, a stem cell growth factor, a lymphotoxin, a hematopoietic factor, a colony stimulating factor (CSF), an interferon (IFN), erythropoietin, thrombopoietin and combinations thereof. Exemplary immunomodulators may include IL-1, IL-2, IL-3, IL-6, IL-10, IL-12, IL-18, IL-21, interferon-a, interferon-β, interferon-γ, G-CSF, GM-CSF, and mixtures thereof.

Exemplary anti-angiogenic agents may include angiostatin, endostatin, baculostatin, canstatin, maspin, anti-VEGF binding molecules, anti-placental growth factor binding molecules, or anti-vascular growth factor binding molecules.

In certain embodiments, the antibody or fragment may comprise one or more chelating moieties, such as NOTA, DOTA, DTPA, TETA, Tscg-Cys, or Tsca-Cys. In certain embodiments, the chelating moiety may form a complex with a therapeutic or diagnostic cation, such as Group II, Group III, Group IV, Group V, transition, lanthanide or actinide metal cations, Tc, Re, Bi, Cu, As, Ag, Au, At, or Pb.

In some embodiments, the antibody or fragment thereof may be a human, chimeric, or humanized antibody or fragment thereof. A humanized antibody or fragment thereof may comprise the complementarity-determining regions (CDRs) of a murine antibody and the constant and framework (FR) region sequences of a human antibody, which may be substituted with at least one amino acid from corresponding FRs of a murine antibody. A chimeric antibody or fragment thereof may include the light and heavy chain variable regions of a murine antibody, attached to human antibody constant regions. The antibody or fragment thereof may include human constant regions of IgG1, IgG2a, IgG3, or IgG4.

Exemplary known antibodies that may be used in combination with anti-CD20/CD74 include, but are not limited to, hR1 (anti-IGF-1R), hPAM4 (anti-mucin), hA19 (anti-CD19), hIMMU31 (anti-AFP), hLL2 (anti-CD22), hMu-9 (anti-CSAp), hL243 (anti-HLA-DR), hMN-14 (anti-CEACAM5), hMN-15 (anti-CEACAM6), 29H2 (anti-CEACAM1, ABCAM®), hRS7 (anti-EGP-1) and hMN-3 (anti-CEACAM6).

In alternative embodiments the anti-CD74 and anti-CD20 antibodies or fragments may be used in combination with another antibody or fragment thereof that binds to one or more target antigens selected from the group consisting of carbonic anhydrase IX, alpha-fetoprotein, α-actinin-4, A3, antigen specific for A33 antibody, ART-4, B7, Ba 733, BAGE, BrE3-antigen, CA125, CAMEL, CAP-1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD132, CD133, CD138, CD147, CD154, CDC27, CDK-4/m, CDKN2A, CXCR4, colon-specific antigen-p (CSAp), CEA (CEACAM5), CEACAM1, CEACAM6, c-met, DAM, EGFR, EGFRvIII, EGP-1, EGP-2, ELF2-M, Ep-CAM, Flt-1, Flt-3, folate receptor, G250 antigen, GAGE, gp100, GROB, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5, MUM-1/2, MUM-3, NCA66, NCA95, NCA90, antigen specific for PAM-4 antibody, placental growth factor, p53, PLAGL2, prostatic acid phosphatase, PSA, PRAIVIE, PSMA, P1GF, IGF, IGF-1R, IL-6, RS5, RANTES, T101, SAGE, 5100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, cMET, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207). Reports on tumor associated antigens include Mizukami et al., (2005, Nature Med. 11:992-97); Hatfield et al., (2005, Curr. Cancer Drug Targets 5:229-48); Vallbohmer et al. (2005, J. Clin. Oncol. 23:3536-44); and Ren et al. (2005, Ann. Surg. 242:55-63).

In certain embodiments, the combination of anti-CD74 and anti-CD20 antibodies or fragments thereof may comprise a DOCK-AND-LOCK® (DNL®) construct (see, e.g., U.S. Pat. Nos. 7,521,056; 7,527,787; 7,534,866; 7,550,143; 7,666,400; 7,858,070; 7,871,622; 7,901,680; 7,906,118 and 7,906,121, the Examples section of each of which is incorporated herein by reference.) The DNL® technique takes advantage of the specific, high-affinity binding interaction between a dimerization and docking domain (DDD) sequence from the regulatory subunit of human cAMP-dependent protein kinase (PKA), such as human PKA RIα, RIβ, RIIα or RIIβ, and an anchor domain (AD) sequence from any of a variety of AKAP proteins. The DDD and AD peptides may be attached to any protein, peptide or other molecule. Because the DDD sequences spontaneously dimerize and bind to the AD sequence, the DNL® technique allows the formation of complexes between any selected molecules that may be attached to DDD or AD sequences. Although the standard DNL® complex comprises a trimer with two DDD-linked molecules attached to one AD-linked molecule, variations in complex structure allow the formation of dimers, trimers, tetramers, pentamers, hexamers and other multimers. In some embodiments, the DNL® complex may comprise two or more antibodies, antibody fragments or fusion proteins which bind to the same antigenic determinant or to two or more different antigens. The DNL® complex may also comprise one or more other effectors, such as a cytokine, toxin or PEG moiety. In preferred embodiments, hexavalent DNL® constructs comprise an IgG molecule covalently attached to two copies of an AD moiety, which binds to four Fab fragments, each covalently attached to a DDD moiety. By formation of disulfide bonds between the AD and DDD subunits, the entire DNL® complex is highly stable under in vivo conditions and each Fab moiety retains the binding specificity and affinity of the parent antibody.

### BRIEF DESCRIPTION OF THE DRAWINGS

The following Figures are provided to illustrate exemplary, but non-limiting, preferred embodiments of the invention.

FIG. 1A. Kaplan-Meier curve demonstrating the progression free survival (PFS) in patients receiving combined veltuzumab and milatuzumab. PFS was calculated from the start of treatment to disease progression. Patients who were still disease free were censored at the date of last disease-free follow up. Survival curves were estimated using the method of Kaplan-Meier. Estimated median with 95% confidence intervals are reported in the Table.

FIG. 1B. Kaplan-Meier curve demonstrating the overall survival in patients receiving combined veltuzumab and milatuzumab. Overall survival (OS) was determined from the start of treatment to death from any cause. Patients who were still alive were censored at the date of last follow-up. Survival curves were estimated using the method of Kaplan-Meier. Estimated median with 95% confidence intervals are reported in the Table.

FIG. 2. Semilog plots of serum veltuzumab concentration versus time at fourth infusion (week-4) for patients with evaluable PK profiles (n=22). Atypical PK profiles were observed in six patients (♦). One patient (x) developed human anti-veltuzumab antibodies (HAHA), atypical PK profile is noted.

FIG. 3 Semilog plots of serum milatuzumab concentration versus time at 1^{st} infusion for patients dosed at 20 mg/kg with evaluable PK profiles (n=17). Linear y-axis is displayed in the upper right corner.

FIG. 4A. Direct cytotoxicity induced by anti-CD20/CD74 HexAbs in NHL cell lines as determined by the MTS assay. JeKo-1, Granta-519, Mino, and Raji (5×10⁴ cells per well in 48-well plate) treated with indicated concentrations of antibodies for 4 days.

FIG. 4B. Direct cytotoxicity induced by anti-CD20/CD74 HexAbs in NHL cell lines as determined by the MTS assay. Effect of monospecific 20-(20)-(20) on JeKo-1, Granta-519 and Mino; bispecific 20-(22)-(22) on JeKo-1; and monospecific 74-(74)-(74) on Raji.

FIG. 4C. Direct cytotoxicity induced by anti-CD20/CD74 HexAbs in NHL cell lines as determined by the MTS assay. Dose-response curves showing partial inhibition of 20-(74)-(74) and 74-(20)-(20) in JeKo-1 by excess hA20 or hLL1 (10 μg/ml).

FIG. 5A. Induction of apoptosis by anti-CD20/CD74 HexAbs. JeKo-1 cells (2×10⁵ cells per well in 6-well plate) were treated with 10 nM of indicated antibodies for 48 h followed by annexin staining analysis. The two bispecific anti-CD20/CD74 HexAbs induced statistically significant apoptosis in JeKo-1 cells compared to cells treated or not treated with parental antibodies, alone or combined (P<0.033).

FIG. 5B. Induction of apoptosis by anti-CD20/CD74 HexAbs. Annexin analysis on primary samples from MCL patients treated with indicated antibodies (10 nM) for 24 to 48 h. Data are shown as Annexin⁺PI⁻ cells (early apoptosis). The two anti-CD20/CD74 HexAbs induced statistically significant early apoptosis in MCL (P<0.008) and CLL (P<0.03) compared to the untreated controls. One of the patient samples (CLL 216) did not respond to any treatment.

FIG. 5C. Induction of apoptosis by anti-CD20/CD74 HexAbs. Annexin analysis on primary samples from CLL patients treated with indicated antibodies (10 nM) for 24 to 48 h. Data are shown as Annexin⁺PI⁻ cells (early apoptosis). The two anti-CD20/CD74 HexAbs induced statistically significant early apoptosis in MCL (P<0.008) and CLL (P<0.03) compared to the untreated controls. One of the patient samples (CLL 216) did not respond to any treatment.

FIG. 5D. Induction of apoptosis by anti-CD20/CD74 HexAbs. Both anti-CD20/CD74 HexAbs induced changes in mitochondrial membrane potential (upper panel) and generated ROS (lower panel) in Granta-519.

FIG. 6A. Correlation of homotypic adhesion, actin reorganization, and lysosomal involvement to cell death evoked by the bispecific anti-CD20/CD74 HexAbs. Apoptosis induced by HexAbs was reduced significantly (P<0.025) in Jeko-1 with 2 μM of cytochalasin D (CsD), another inhibitor of actin polymerization.

FIG. 6B. Correlation of homotypic adhesion, actin reorganization, and lysosomal involvement to cell death evoked by the bispecific anti-CD20/CD74 HexAbs. Lysosomal V ATPase inhibitors, concanamycin A (Con A) and bafilomycin A1 (Bfa1), inhibited the apoptosis induced by HexAbs in JeKo-1 cells.

FIG. 7A. Activity of HexAbs in human blood ex vivo. 20-(74)-(74) and 74-(20)-(20), tested at 10 and 25 nM in JeKo-1 (upper panel) and normal B cells (lower panel). The effect of the indicated antibodies on the growth of spiked JeKo-1 cells in whole blood from a healthy volunteer was determined after 48 h. JeKo-1 cells were analyzed as CD19+ events in the monocyte gate. B cells were analyzed as CD19+ events in the lymphocyte gate. Error bars represent SD.

FIG. 7B. Activity of HexAbs in human blood ex vivo. 20-(74)-(74) tested at 0.1, 0.5 and 1 nM in Jeko-1 (upper panel) and normal B cells (lower panel). The effect of the indicated antibodies on the growth of spiked JeKo-1 cells in whole blood from a healthy volunteer was determined after 48 h. JeKo-1 cells were analyzed as CD19+ events in the monocyte gate. B cells were analyzed as CD19+ events in the lymphocyte gate. Error bars represent SD

FIG. 7C. Activity of HexAbs in human blood ex vivo. 74-(20)-(20) tested at 0.1, 0.5 and 1 nM in JeKo-1 (upper panel) and normal B cells (lower panel). The effect of the indicated antibodies on the growth of spiked JeKo-1 cells in whole blood from a healthy volunteer was determined after 48 h. JeKo-1 cells were analyzed as CD19+ events in the monocyte gate. B cells were analyzed as CD19+ events in the lymphocyte gate. Error bars represent SD.

FIG. 8. Therapeutic efficacy of HexAbs in disseminated JeKo-1 xenograft model. Seven groups of 8 mice (8-wk-old female SCID mice) each were inoculated i.v. with JeKo-1 (2.5×10⁷ cells per animal). After 7 days, three different does (i.e., 37 μg, 37 μg and 3.7 μg) of both HexAbs were administered via i.p. injections twice a week for two weeks. Control mice received saline injections. 74-(20)-(20) and 20-(74)-(74), at the 370 μg dose level, resulted in 30% and 60% increases in median survival compared to saline controls, respectively.

FIG. 9. 20-(74)-(74) Induced potent ADCC in JeKo-1 cells.

FIG. 10. Anti-CD74 antibody (milatuzumab) increases the cytotoxicity of rituximab. The Figure shows the percent of untreated control values in MTT cytotoxicity assays. Cells were incubated with the antibodies for 4 days in the presence of goat anti-human IgG as a crosslinker. The Figure shows several lines of NHL (left side), CLL (center) and ALL (right side) cell lines.

FIG. 11. Anti-CD74 antibody (milatuzumab) increases the cytotoxicity of fludarabine. The Figure shows the percent of untreated control values in MTS cytotoxicity assays. Cells were incubated with fludarabine and milatuzumab for 4 days in the presence of goat anti-human IgG as a crosslinker. The Figure shows the effects on NHL (left side) and CLL (right side) cell lines.

### DEFINITIONS

The following definitions are provided to facilitate understanding of the disclosure herein. Where a term is not specifically defined, it is used in accordance with its plain and ordinary meaning.

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody).

An “antibody fragment” is a portion of an antibody such as F(ab′)₂, F(ab)₂, Fab′, Fab, Fv, scFv, single domain antibodies (DABs or VHHs) and the like, including half-molecules of IgG4 (van der Neut Kolfschoten et al., 2007, Science 317:1554-1557). Regardless of structure, an antibody fragment binds with the same antigen that is recognized by the intact antibody. For example, an anti-CD22 antibody fragment binds with an epitope of CD22. The term “antibody fragment” also includes isolated fragments consisting of the variable regions, such as the “Fv” fragments consisting of the variable regions of the heavy and light chains, recombinant single chain polypeptide molecules in which light and heavy chain variable regions are connected by a peptide linker (“scFv proteins”), and minimal recognition units consisting of the amino acid residues that mimic the hypervariable region.

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains, including human framework region (FR) sequences. The constant domains of the antibody molecule are derived from those of a human antibody.

A “human antibody” is an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, cytokine or chemokine inhibitors, pro-apoptotic agents, tyrosine kinase inhibitors, toxins, enzymes, nucleases, hormones, immunomodulators, antisense oligonucleotides, siRNA, RNAi, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

An “immunoconjugate” is a conjugate of an antibody with an atom, molecule, or a higher-ordered structure (e.g., with a liposome), a therapeutic agent, or a diagnostic agent.

A “naked antibody” is generally an entire antibody that is not conjugated to a therapeutic agent. This is so because the Fc portion of the antibody molecule provides effector functions, such as complement fixation and ADCC (antibody dependent cell cytotoxicity) that set mechanisms into action that may result in cell lysis. However, it is possible that the Fc portion is not required for therapeutic function, with other mechanisms, such as apoptosis, coming into play. Naked antibodies include both polyclonal and monoclonal antibodies, as well as certain recombinant antibodies, such as chimeric, humanized or human antibodies.

As used herein, the term “antibody fusion protein” is a recombinantly produced antigen-binding molecule in which an antibody or antibody fragment is linked to another protein or peptide, such as the same or different antibody or antibody fragment or a DDD or AD peptide (of the DOCK-AND-LOCK® complexes described below). The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity.

A “bispecific antibody” is an antibody that can bind simultaneously to two targets which are of different structure. Bispecific antibodies (bsAb) and bispecific antibody fragments (bsFab) may have at least one arm that specifically binds to, for example, a B cell, T cell, myeloid-, plasma-, and mast-cell antigen or epitope and at least one other arm that specifically binds to a targetable conjugate that bears a therapeutic or diagnostic agent. A variety of bispecific antibodies can be produced using molecular engineering. Included herein are bispecific antibodies that target a cancer-associated antigen and also an immunotherapeutic T cell, such as CD3-T cells.

The term “direct cytotoxicity” refers to the ability of an agent to inhibit the proliferation or induce the apoptosis of a cell grown in an optimized culture medium in which only the agent and the cell are present.

Monoclonal Antibodies

The compositions, formulations and methods described herein may include monoclonal antibodies. Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)). General techniques for cloning murine immunoglobulin variable domains have been disclosed, for example, by the publication of Orlandi et al., Proc. Nat'l Acad. Sci. USA 86: 3833 (1989).

Chimeric Antibodies

A chimeric antibody is a recombinant protein that contains the variable domains including the CDRs derived from one species of animal, such as a rodent antibody, while the remainder of the antibody molecule; i.e., the constant domains, is derived from a human antibody. Techniques for constructing chimeric antibodies are well known to those of skill in the art. As an example, Leung et al., Hybridoma 13:469 (1994), disclose how they produced an LL2 chimera by combining DNA sequences encoding the Vₖ and V_{H} domains of LL2 monoclonal antibody, an anti-CD22 antibody, with respective human and IgG₁ constant region domains. This publication also provides the nucleotide sequences of the LL2 light and heavy chain variable regions, Vₖ and V_{H}, respectively.

Humanized Antibodies

A chimeric monoclonal antibody can be humanized by replacing the sequences of the murine FR in the variable domains of the chimeric antibody with one or more different human FR. Specifically, mouse CDRs are transferred from heavy and light variable chains of the mouse immunoglobulin into the corresponding variable domains of a human antibody. As simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. (See, e.g., Tempest et al., Biotechnology 9:266 (1991) and Verhoeyen et al., Science 239: 1534 (1988)). Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

Human Antibodies

A fully human antibody can be obtained from a transgenic non-human animal. (See, e.g., Mendez et al., Nature Genetics, 15: 146-156, 1997; U.S. Pat. No. 5,633,425.) Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id.). RNAs were converted to cDNAs and used to make Fab cDNA libraries using specific primers against the heavy and light chain immunoglobulin sequences (Marks et al., 1991, J Mol. Biol. 222:581-97). Library construction was performed according to Andris-Widhopf et al. (2000, In: Phage Display Laboratory Manual, Barbas et al. (eds), edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XenoMouse® (e.g., Green et al., 1999, J. Immunol. Methods 231:11-23, incorporated herein by reference) from Abgenix (Fremont, Calif.). In the XenoMouse® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. A XENOMOUSE® immunized with a target antigen will produce human antibodies by the normal immune response, which may be harvested and/or produced by standard techniques discussed above. A variety of are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

Antibody Cloning and Production

Various techniques, such as production of chimeric or humanized antibodies, may involve procedures of antibody cloning and construction. The antigen-binding V_{κ} (variable light chain) and V_{H} (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned V_{L} and V_{H} genes can be expressed in cell culture as a chimeric Ab as described by Orlandi et al., (Proc. Natl. Acad. Sci., USA, 86: 3833 (1989)). Based on the V gene sequences, a humanized antibody can then be designed and constructed as described by Leung et al. (Mol. Immunol., 32: 1413 (1995)). cDNA can be prepared from any known hybridoma line or transfected cell line producing a murine antibody by general molecular cloning techniques (Sambrook et al., Molecular Cloning, A laboratory manual, 2^{nd} Ed (1989)). The V_{κ} sequence for the antibody may be amplified using the primers VK1BACK and VK1FOR (Orlandi et al., 1989) or the extended primer set described by Leung et al. (BioTechniques, 15: 286 (1993)). The V_{H} sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for Vκ can be subcloned into a staging vector, such as a pBR327-based staging vector, VKpBR, that contains an Ig promoter, a signal peptide sequence and convenient restriction sites. PCR products for V_{H} can be subcloned into a similar staging vector, such as the pBluescript-based VHpBS. Expression cassettes containing the Vκ and V_{H} sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the Vκ and V_{H} expression cassettes can be excised and subcloned into a single expression vector, such as pdHL2, as described by Gillies et al. (J. Immunol. Methods 125:191 (1989) and also shown in Losman et al., Cancer, 80:2660 (1997)).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930 and 7,608,425; the Examples section of each of which is incorporated herein by reference). These exemplary cell lines are based on the Sp2/0 myeloma cell line, transfected with a mutant Bcl-EEE gene, exposed to methotrexate to amplify transfected gene sequences and pre-adapted to serum-free cell line for protein expression.

Antibody Allotypes

Immunogenicity of therapeutic antibodies is associated with increased risk of infusion reactions and decreased duration of therapeutic response (Baert et al., 2003, N Engl J Med 348:602-08). The extent to which therapeutic antibodies induce an immune response in the host may be determined in part by the allotype of the antibody (Stickler et al., 2011, Genes and Immunity 12:213-21). Antibody allotype is related to amino acid sequence variations at specific locations in the constant region sequences of the antibody. The allotypes of IgG antibodies containing a heavy chain γ-type constant region are designated as Gm allotypes (1976, J Immunol 117:1056-59). For the common IgG1 human antibodies, the most prevalent allotype is G1m1 (Stickler et al., 2011, Genes and Immunity 12:213-21). However, the G1m3 allotype also occurs frequently in Caucasians (Id.). It has been reported that G1m1 antibodies contain allotypic sequences that tend to induce an immune response when administered to non-G1m1 (nG1 m1) recipients, such as G1m3 patients (Id.). Non-G1m1 allotype antibodies are not as immunogenic when administered to G1m1 patients (Id.).

The human G1m1 allotype comprises the amino acids aspartic acid at Kabat position 356 and leucine at Kabat position 358 in the CH3 sequence of the heavy chain IgG1. The nG1m1 allotype comprises the amino acids glutamic acid at Kabat position 356 and methionine at Kabat position 358. Both G1m1 and nG1m1 allotypes comprise a glutamic acid residue at Kabat position 357 and the allotypes are sometimes referred to as DEL and EEM allotypes. A non-limiting example of the heavy chain constant region sequences for G1m1 and nG1m1 allotype antibodies is shown for the exemplary antibodies rituximab (SEQ ID NO:14) and veltuzumab (SEQ ID NO:13).

Veltuzumab heavy chain constant region sequence

(SEQ ID NO: 13)

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Rituximab heavy chain constant region sequence

(SEQ ID NO: 14)

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Jefferis and Lefranc (2009, mAbs 1:1-7) reviewed sequence variations characteristic of IgG allotypes and their effect on immunogenicity. They reported that the G1m3 allotype is characterized by an arginine residue at Kabat position 214, compared to a lysine residue at Kabat 214 in the G1m17 allotype. The nG1 m1,2 allotype was characterized by glutamic acid at Kabat position 356, methionine at Kabat position 358 and alanine at Kabat position 431. The G1m1,2 allotype was characterized by aspartic acid at Kabat position 356, leucine at Kabat position 358 and glycine at Kabat position 431. In addition to heavy chain constant region sequence variants, Jefferis and Lefranc (2009) reported allotypic variants in the kappa light chain constant region, with the Km1 allotype characterized by valine at Kabat position 153 and leucine at Kabat position 191, the Km1,2 allotype by alanine at Kabat position 153 and leucine at Kabat position 191, and the Km3 allotype characterized by alanine at Kabat position 153 and valine at Kabat position 191.

With regard to therapeutic antibodies, veltuzumab and rituximab are, respectively, humanized and chimeric IgG1 antibodies against CD20, of use for therapy of a wide variety of hematological malignancies and/or autoimmune diseases. Table 1 compares the allotype sequences of rituximab vs. veltuzumab. As shown in Table 1, rituximab (G1m17,1) is a DEL allotype IgG1, with an additional sequence variation at Kabat position 214 (heavy chain CH1) of lysine in rituximab vs. arginine in veltuzumab. It has been reported that veltuzumab is less immunogenic in subjects than rituximab (see, e.g., Morchhauser et al., 2009, J Clin Oncol 27:3346-53; Goldenberg et al., 2009, Blood 113:1062-70; Robak & Robak, 2011, BioDrugs 25:13-25), an effect that has been attributed to the difference between humanized and chimeric antibodies. However, the difference in allotypes between the EEM and DEL allotypes likely also accounts for the lower immunogenicity of veltuzumab.

TABLE 1
Allotypes of Rituximab vs. Veltuzumab

| | | Heavy chain position and associated allotypes | | | | | |
|---|---|---|---|---|---|---|---|
| | Complete | 214 | | 356/358 | | 431 | |
| | allotype | (allotype) | | (allotype) | | (allotype) | |
| Rituximab | G1m17,1 | K | 17 | D/L | 1 | A | — |
| Veltuzumab | G1m3 | R | 3 | E/M | — | A | — |

In order to reduce the immunogenicity of therapeutic antibodies in individuals of nG1m1 genotype, it is desirable to select the allotype of the antibody to correspond to the G1m3 allotype, characterized by arginine at Kabat 214, and the nG1 m1,2 null-allotype, characterized by glutamic acid at Kabat position 356, methionine at Kabat position 358 and alanine at Kabat position 431. Surprisingly, it was found that repeated subcutaneous administration of G1m3 antibodies over a long period of time did not result in a significant immune response. In alternative embodiments, the human IgG4 heavy chain in common with the G1m3 allotype has arginine at Kabat 214, glutamic acid at Kabat 356, methionine at Kabat 359 and alanine at Kabat 431. Since immunogenicity appears to relate at least in part to the residues at those locations, use of the human IgG4 heavy chain constant region sequence for therapeutic antibodies is also a preferred embodiment. Combinations of G1m3 IgG1 antibodies with IgG4 antibodies may also be of use for therapeutic administration.

Known Antibodies

In various embodiments, the claimed methods and compositions may utilize any of a variety of antibodies known in the art. For example, therapeutic use of anti-CD74/CD20 antibodies may be supplemented with one or more antibodies against other tumor-associated antigens. Antibodies of use may be commercially obtained from a number of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572; 856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206′ 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

Antibodies of use may bind to various known antigens expressed in B cells, including but not limited to BCL-1, BCL-2, BCL-6, CD1a, CD2, CD5, CD10, CD11b, CD11c, CD13, CD14, CD15, CD19, CD20, CD21, CD22, CD23, CD25, CD34, CD38, CD40, CD40L, CD41a, CD43, CD45, CD47, CD55, CD56, CCD57, CD59, CD64, CD71, CD79a, CD79b, CD138, CXCR4, FMC-7 and HLA-DR.

Particular antibodies that may be of use for therapy of cancer within the scope of the claimed methods and compositions include, but are not limited to, LL1 (anti-CD74), LL2 and RFB4 (anti-CD22), hL243 (anti-HLA-DR), alemtuzumab (anti-CD52), gemtuzumab (anti-CD33), ibritumomab (anti-CD20), rituximab (anti-CD20), tositumomab (anti-CD20), and GA101 (anti-CD20; obinutuzumab). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20040202666 (now abandoned); 20050271671; and 20060193865; the Examples section of each incorporated herein by reference.) Specific known antibodies of use include hA20 (U.S. Pat. No. 7,151,164), hA19 (U.S. Pat. No. 7,109,304), hLL1 (U.S. Pat. No. 7,312,318), hLL2 (U.S. Pat. No. 7,074,403), and hL243 (U.S. Pat. No. 7,612,180), the text of each recited patent or application is incorporated herein by reference with respect to the Figures and Examples sections.

Antibody Fragments

Antibody fragments which recognize specific epitopes can be generated by known techniques. The antibody fragments are antigen binding portions of an antibody, such as F(ab)₂, Fab′, Fab, Fv, scFv and the like. Other antibody fragments include, but are not limited to: the F(ab′)₂ fragments which can be produced by pepsin digestion of the antibody molecule and the Fab′ fragments, which can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity. In certain embodiments, the antibody fragment may be a fragment that is not an scFv fragment.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991).

An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

A single complementarity-determining region (CDR) is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. (See, e.g., Larrick et al., Methods: A Companion to Methods in Enzymology 2: 106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780).

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797; Hwang and Foote, 2005, Methods 36:3-10; Clark, 2000, Immunol Today 21:397-402; J Immunol 1976 117:1056-60; Ellison et al., 1982, Nucl Acids Res 13:4071-79; Stickler et al., 2011, Genes and Immunity 12:213-21).

Multispecific and Multivalent Antibodies

Methods for producing bispecific antibodies include engineered recombinant antibodies which have additional cysteine residues so that they crosslink more strongly than the more common immunoglobulin isotypes. (See, e.g., FitzGerald et al, Protein Eng. 10(10):1221-1225, (1997)). Another approach is to engineer recombinant fusion proteins linking two or more different single-chain antibody or antibody fragment segments with the needed dual specificities. (See, e.g., Coloma et al., Nature Biotech. 15:159-163, (1997)). A variety of bispecific antibodies can be produced using molecular engineering. In one form, the bispecific antibody may consist of, for example, an scFv with a single binding site for one antigen and a Fab fragment with a single binding site for a second antigen. In another form, the bispecific antibody may consist of, for example, an IgG with two binding sites for one antigen and two scFv with two binding sites for a second antigen. In alternative embodiments, multispecific and/or multivalent antibodies may be produced as DOCK-AND-LOCK® (DNL®) complexes as described below.

In certain embodiments, an anti-CD74 and anti-CD20 antibody or fragment may be administered to a patient as part of a combination of antibodies. Bispecific antibodies are preferred to administration of combinations of separate antibodies, due to cost and convenience. However, where combinations of separate antibodies provide improved safety or efficacy, the combination may be utilized. The antibodies may bind to different epitopes of the same antigen or to different antigens. Preferably, the antigens are selected from the group consisting of BCL-1, BCL-2, BCL-6, CD1a, CD2, CD5, CD10, CD11b, CD11c, CD13, CD14, CD15, CD19, CD20, CD21, CD22, CD23, CD25, CD34, CD38, CD40, CD40L, CD41a, CD43, CD45, CD47, CD55, CD56, CCD57, CD59, CD64, CD71, CD79a, CD79b, CD138, CXCR4, FMC-7 and HLA-DR.

Dock-and-Lock® (DNL®)

In preferred embodiments, a bivalent or multivalent antibody is formed as a DOCK-AND-LOCK® (DNL®) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264. Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Although the standard DNL® complex comprises a trimer with two DDD-linked molecules attached to one AD-linked molecule, variations in complex structure allow the formation of dimers, trimers, tetramers, pentamers, hexamers and other multimers. In some embodiments, the DNL® complex may comprise two or more antibodies, antibody fragments or fusion proteins which bind to the same antigenic determinant or to two or more different antigens. The DNL® complex may also comprise one or more other effectors, such as proteins, peptides, immunomodulators, cytokines, interleukins, interferons, binding proteins, peptide ligands, carrier proteins, toxins, ribonucleases such as onconase, inhibitory oligonucleotides such as siRNA, antigens or xenoantigens, polymers such as PEG, enzymes, therapeutic agents, hormones, cytotoxic agents, anti-angiogenic agents, pro-apoptotic agents or any other molecule or aggregate.

PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has a and _{R} isoforms (Scott, Pharmacol. Ther. 1991; 50:123). Thus, the four isoforms of PKA regulatory subunits are RIₐ, RIIβ, RIIα and RIIβ. The R subunits have been isolated only as stable dimers and the dimerization domain has been shown to consist of the first 44 amino-terminal residues of RIIα or RIIβ (Newlon et al., Nat. Struct. Biol. 1999; 6:222). As discussed below, similar portions of the amino acid sequences of other regulatory subunits are involved in dimerization and docking, each located at or near the N-terminal end of the regulatory subunit. Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561)

Since the first AKAP, microtubule-associated protein-2, was characterized in 1984 (Lohmann et al., Proc. Natl. Acad. Sci USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIα are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human PKA regulatory subunits and the AD of AKAP as an excellent pair of linker modules for docking any two entities, referred to hereafter as A and B, into a noncovalent complex, which could be further locked into a DNL® complex through the introduction of cysteine residues into both the DDD and AD at strategic positions to facilitate the formation of disulfide bonds. The general methodology of the approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL® constructs of different stoichiometry may be produced and used (see, e.g., U.S. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL® construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

Structure-Function Relationships in AD and DDD Moieties

For different types of DNL® constructs, different AD or DDD sequences may be utilized. Exemplary DDD and AD sequences are provided below.

DDD1

(SEQ ID NO: 15)

SHIQIPPGLTELLQGYTVEVLRQQPPDLVEFAVEYFTRLREARA
DDD2

(SEQ ID NO: 16)

CGHIQIPPGLTELLQGYTVEVLRQQPPDLVEFAVEYFTRLREARA
AD1

(SEQ ID NO: 17)

QIEYLAKQIVDNAIQQA
AD2

(SEQ ID NO: 18)

CGQIEYLAKQIVDNAIQQAGC

The skilled artisan will realize that DDD1 and DDD2 are based on the DDD sequence of the human RIIα isoform of protein kinase A. However, in alternative embodiments, the DDD and AD moieties may be based on the DDD sequence of the human Ma form of protein kinase

A and a corresponding AKAP sequence, as exemplified in DDD3, DDD3C and AD3 below.

DDD3
(SEQ ID NO: 19)
SLRECELYVQKHNIQALLKDSIVQLCTARPERPMAFLREYFERLEKEEAK
DDD3C
(SEQ ID NO: 20)
MSCGGSLRECELYVQKHNIQALLKDSIVQLCTARPERPMAFLREYFERLE
KEEAK
AD3
(SEQ ID NO: 21)
CGFEELAWKIAKMIWSDVFQQGC

In other alternative embodiments, other sequence variants of AD and/or DDD moieties may be utilized in construction of the DNL® complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 22)
SLRECELYVQKHNIQALLKDVSIVQLCTARPERPMAFLREYFEKLEKEE
AK
PKA RIβ
(SEQ ID NO: 23)
SLKGCELYVQLHGIQQVLKDCIVHLCISKPERPMKFLREHFEKLEKEENR
QILA
PKA RIIα
(SEQ ID NO: 24)
SHIQIPPGLTELLQGYTVEVGQQPPDLVDFAVEYFTRLREARRQ
PKA RIIβ
(SEQ ID NO: 25)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Carr et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

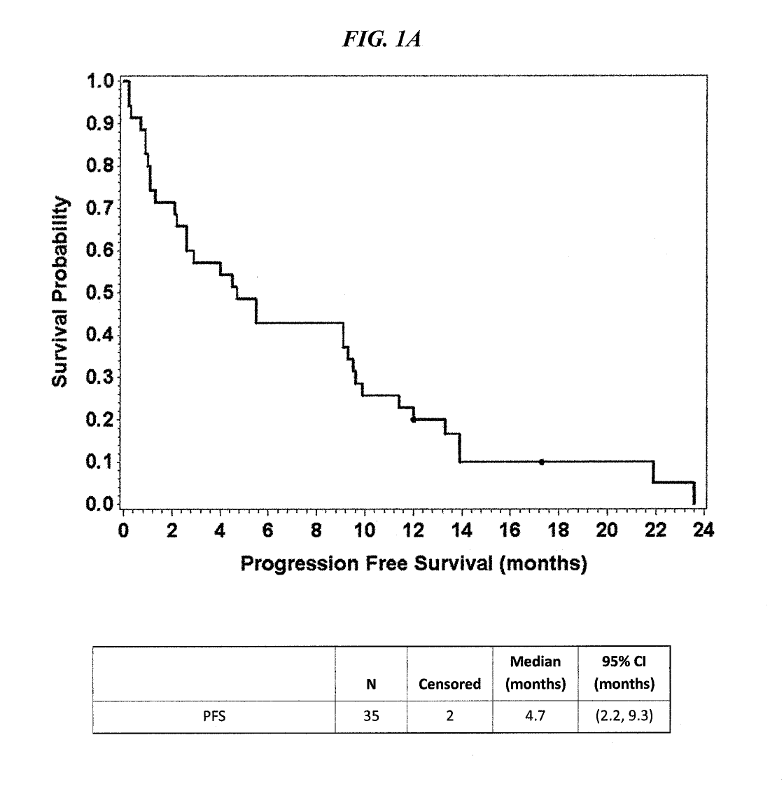

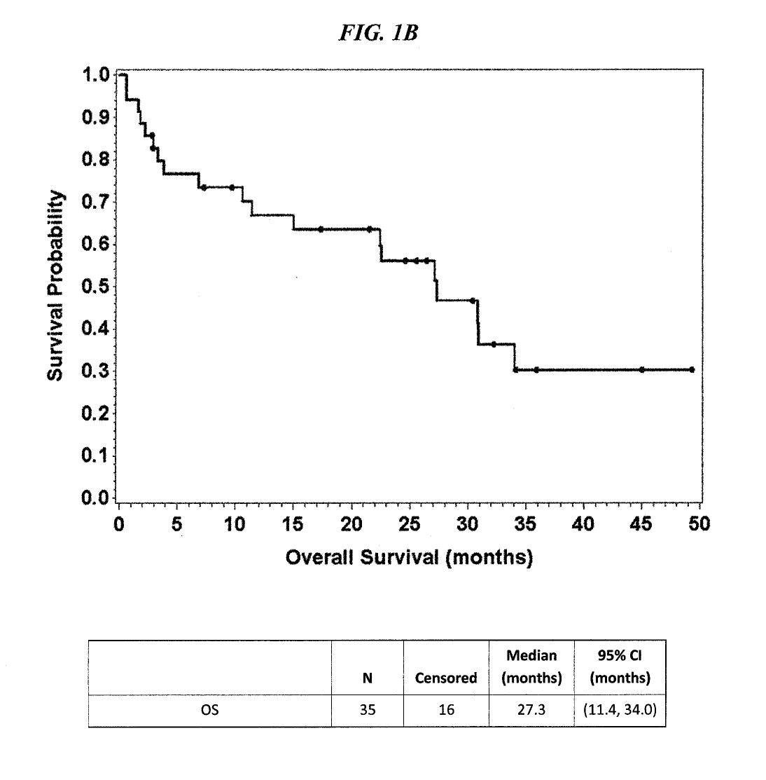

For example, Kinderman et al. (2006, Mol Cell 24:397-408) examined the crystal structure of the AD-DDD binding interaction and concluded that the human DDD sequence contained a number of conserved amino acid residues that were important in either dimer formation or AKAP binding, underlined in SEQ ID NO:15 below. (See FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

(SEQ ID NO: 15)

SHIQIPPGLTELLQGYTVEVLRQQPPDLVEFAVEYFTRLREARA

As discussed in more detail below, conservative amino acid substitutions have been characterized for each of the twenty common L-amino acids. Thus, based on the data of Kinderman (2006) and conservative amino acid substitutions, potential alternative DDD sequences based on SEQ ID NO:15 are shown in Table 2. In devising Table 2, only highly conservative amino acid substitutions were considered. For example, charged residues were only substituted for residues of the same charge, residues with small side chains were substituted with residues of similar size, hydroxyl side chains were only substituted with other hydroxyls, etc. Because of the unique effect of proline on amino acid secondary structure, no other residues were substituted for proline. A limited number of such potential alternative DDD moiety sequences are shown in SEQ ID NO:27 to SEQ ID NO:46 below. The skilled artisan will realize that an almost unlimited number of alternative species within the genus of DDD moieties can be constructed by standard techniques, for example using a commercial peptide synthesizer or well known site-directed mutagenesis techniques. The effect of the amino acid substitutions on AD moiety binding may also be readily determined by standard binding assays, for example as disclosed in Alto et al. (2003, Proc Natl Acad Sci USA 100:4445-50).

TABLE 2
Conservative Amino Acid Substitutions in DDD1
(SEQ ID NO: 15). Consensus sequence disclosed
as SEQ ID NO: 26.

| S | H | I | Q | I | P | P | G | L | T | E | L | L | Q | G | Y | T | V | E | V | L | R |
|---|---|---|---|---|---|---|---|---|---|---|---|---|---|---|---|---|---|---|---|---|---|
| T | K | | N | | | | A | | S | D | | | N | A | | S | | D | | | K |
| | R | | | | | | | | | | | | | | | | | | | | |
| Q | Q | P | P | D | L | V | E | F | A | V | E | Y | F | T | R | L | R | E | A | R | A |
| N | N | | | E | | | D | | L | | D | | | S | K | | K | D | L | K | L |
| | | | | | | | | | I | | | | | | | | | | I | | I |
| | | | | | | | | | V | | | | | | | | | | V | | V |

(SEQ ID NO: 27)

THIQIPPGLTELLQGYTVEVLRQQPPDLVEFAVEYFTRLREARA

(SEQ ID NO: 28)

SKIQIPPGLTELLQGYTVEVLRQQPPDLVEFAVEYFTRLREARA

(SEQ ID NO: 29)

SRIQIPPGLTELLQGYTVEVLRQQPPDLVEFAVEYFTRLREARA

(SEQ ID NO: 30)

SHINIPPGLTELLQGYTVEVLRQQPPDLVEFAVEYFTRLREARA

(SEQ ID NO: 31)

SHIQIPPALTELLQGYTVEVLRQQPPDLVEFAVEYFTRLREARA

(SEQ ID NO: 32)

SHIQIPPGLSELLQGYTVEVLRQQPPDLVEFAVEYFTRLREARA

(SEQ ID NO: 33)

SHIQIPPGLTDLLQGYTVEVLRQQPPDLVEFAVEYFTRLREARA

(SEQ ID NO: 34)

SHIQIPPGLTELLNGYTVEVLRQQPPDLVEFAVEYFTRLREARA

(SEQ ID NO: 35)

SHIQIPPGLTELLQAYTVEVLRQQPPDLVEFAVEYFTRLREARA

(SEQ ID NO: 36)

SHIQIPPGLTELLQGYSVEVLRQQPPDLVEFAVEYFTRLREARA

(SEQ ID NO: 37)

SHIQIPPGLTELLQGYTVDVLRQQPPDLVEFAVEYFTRLREARA

(SEQ ID NO: 38)

SHIQIPPGLTELLQGYTVEVLKQQPPDLVEFAVEYFTRLREARA

(SEQ ID NO: 39)

SHIQIPPGLTELLQGYTVEVLRNQPPDLVEFAVEYFTRLREARA

(SEQ ID NO: 40)

SHIQIPPGLTELLQGYTVEVLRQNPPDLVEFAVEYFTRLREARA

(SEQ ID NO: 41)

SHIQIPPGLTELLQGYTVEVLRQQPPELVEFAVEYFTRLREARA

(SEQ ID NO: 42)

SHIQIPPGLTELLQGYTVEVLRQQPPDLVDFAVEYFTRLREARA

(SEQ ID NO: 43)

SHIQIPPGLTELLQGYTVEVLRQQPPDLVEFLVEYFTRLREARA

(SEQ ID NO: 44)

SHIQIPPGLTELLQGYTVEVLRQQPPDLVEFIVEYFTRLREARA

(SEQ ID NO: 45)

SHIQIPPGLTELLQGYTVEVLRQQPPDLVEFVVEYFTRLREARA

(SEQ ID NO: 46)

SHIQIPPGLTELLQGYTVEVLRQQPPDLVEFAVDYFTRLREARA

Alto et al. (2003, Proc Natl Acad Sci USA 100:4445-50) performed a bioinformatic analysis of the AD sequence of various AKAP proteins to design an RII selective AD sequence called AKAP-IS (SEQ ID NO:17), with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in SEQ ID NO:17 below. The skilled artisan will realize that in designing sequence variants of the AD sequence, one would desirably avoid changing any of the underlined residues, while conservative amino acid substitutions might be made for residues that are less critical for DDD binding. Table 3 shows potential conservative amino acid substitutions in the sequence of AKAP-IS (AD1, SEQ ID NO:17), similar to that shown for DDD1 (SEQ ID NO:15) in Table 2 above.

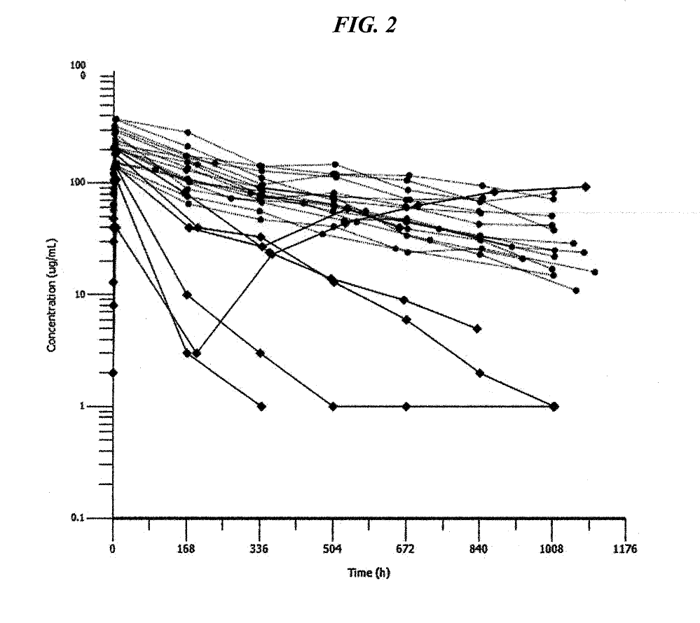

A limited number of such potential alternative AD moiety sequences are shown in SEQ ID NO:47 to SEQ ID NO:64 below. Again, a very large number of species within the genus of possible AD moiety sequences could be made, tested and used by the skilled artisan, based on the data of Alto et al. (2003). It is noted that FIG. 2 of Alto (2003) shows an even large number of potential amino acid substitutions that may be made, while retaining binding activity to DDD moieties, based on actual binding experiments.

AKAP-IS

(SEQ ID NO: 17)

QIEYLAKQIVDNAIQQA

TABLE 3
Conservative Amino Acid Substitutions in AD1
(SEQ ID NO: 17). Consensus sequence disclosed
as SEQ ID NO: 65.

| Q | I | E | Y | L | A | K | Q | I | V | D | N | A | I | Q | Q | A |
|---|---|---|---|---|---|---|---|---|---|---|---|---|---|---|---|---|
| N | L | D | F | I | | R | N | | | E | Q | | | N | N | L |
| | V | | T | V | | | | | | | | | | | | I |
| | | | S | | | | | | | | | | | | | V |

(SEQ ID NO: 47)

NIEYLAKQIVDNAIQQA

(SEQ ID NO: 48)

QLEYLAKQIVDNAIQQA

(SEQ ID NO: 49)

QVEYLAKQIVDNAIQQA

(SEQ ID NO: 50)

QIDYLAKQIVDNAIQQA

(SEQ ID NO: 51)

QIEFLAKQIVDNAIQQA

(SEQ ID NO: 52)

QIETLAKQIVDNAIQQA

(SEQ ID NO: 53)

QIESLAKQIVDNAIQQA

(SEQ ID NO: 54)

QIEYIAKQIVDNAIQQA

(SEQ ID NO: 55)

QIEYVAKQIVDNAIQQA

(SEQ ID NO: 56)

QIEYLARQIVDNAIQQA

(SEQ ID NO: 57)

QIEYLAKNIVDNAIQQA

(SEQ ID NO: 58)

QIEYLAKQIVENAIQQA

(SEQ ID NO: 59)

QIEYLAKQIVDQAIQQA

(SEQ ID NO: 60)

QIEYLAKQIVDNAINQA

(SEQ ID NO: 61)

QIEYLAKQIVDNAIQNA

(SEQ ID NO: 62)

QIEYLAKQIVDNAIQQL

(SEQ ID NO: 63)

QIEYLAKQIVDNAIQQI

(SEQ ID NO: 64)

QIEYLAKQIVDNAIQQV

Gold et al. (2006, Mol Cell 24:383-95) utilized crystallography and peptide screening to develop a SuperAKAP-IS sequence (SEQ ID NO:66), exhibiting a five order of magnitude higher selectivity for the RII isoform of PKA compared with the RI isoform. Underlined residues indicate the positions of amino acid substitutions, relative to the AKAP-IS sequence, which increased binding to the DDD moiety of RIIα. In this sequence, the N-terminal Q residue is numbered as residue number 4 and the C-terminal A residue is residue number 20. Residues where substitutions could be made to affect the affinity for RIIα were residues 8, 11, 15, 16, 18, 19 and 20 (Gold et al., 2006). It is contemplated that in certain alternative embodiments, the SuperAKAP-IS sequence may be substituted for the AKAP-IS AD moiety sequence to prepare DNL® constructs. Other alternative sequences that might be substituted for the AKAP-IS AD sequence are shown in SEQ ID NO:67-69. Substitutions relative to the AKAP-IS sequence are underlined. It is anticipated that, as with the AD2 sequence shown in SEQ ID NO:18, the AD moiety may also include the additional N-terminal residues cysteine and glycine and C-terminal residues glycine and cysteine.

SuperAKAP-IS

(SEQ ID NO: 66)

QIEYVAKQIVDYAIHQA
Alternative AKAP sequences

(SEQ ID NO: 67)

QIEYKAKQIVDHAIHQA

(SEQ ID NO: 68)

QIEYHAKQIVDHAIHQA

(SEQ ID NO: 69)

QIEYVAKQIVDHAIHQA

FIG. 2 of Gold et al. disclosed additional DDD-binding sequences from a variety of AKAP proteins, shown below.

RII-Specific AKAPs
AKAP-KL

(SEQ ID NO: 70)

PLEYQAGLLVQNAIQQAI
AKAP79

(SEQ ID NO: 71)

LLIETASSLVKNAIQLSI
AKAP-Lbc

(SEQ ID NO: 72)

LIEEAASRIVDAVIEQVK
RI-Specific AKAPs
AKAPce

(SEQ ID NO: 73)

ALYQFADRFSELVISEAL
RIAD

(SEQ ID NO: 74)

LEQVANQLADQIIKEAT
PV38

(SEQ ID NO: 75)

FEELAWKIAKMIWSDVF
Dual-Specificity AKAPs
AKAP7

(SEQ ID NO: 76)

ELVRLSKRLVENAVLKAV
MAP2D

(SEQ ID NO: 77)

TAEEVSARIVQVVTAEAV
DAKAP1

(SEQ ID NO: 78)

QIKQAAFQLISQVILEAT
DAKAP2

(SEQ ID NO: 79)

LAWKIAKMIVSDVMQQ

Stokka et al. (2006, Biochem J 400:493-99) also developed peptide competitors of AKAP binding to PKA, shown in SEQ ID NO:80-82. The peptide antagonists were designated as Ht31 (SEQ ID NO:80), RIAD (SEQ ID NO:81) and PV-38 (SEQ ID NO:82). The Ht-31 peptide exhibited a greater affinity for the RII isoform of PKA, while the RIAD and PV-38 showed higher affinity for RI.

Ht31

(SEQ ID NO: 80)

DLIEEAASRIVDAVIEQVKAAGAY
RIAD

(SEQ ID NO: 81)

LEQYANQLADQIIKEATE
PV-38

(SEQ ID NO: 82)

FEELAWKIAKMIWSDVFQQC

Hundsrucker et al. (2006, Biochem J 396:297-306) developed still other peptide competitors for AKAP binding to PKA, with a binding constant as low as 0.4 nM to the DDD of the RII form of PKA. The sequences of various AKAP antagonistic peptides are provided in Table 1 of Hundsrucker et al., reproduced in Table 4 below. AKAPIS represents a synthetic RII subunit-binding peptide. All other peptides are derived from the RII-binding domains of the indicated AKAPs.

TABLE 4
AKAP Peptide sequences

| | Peptide Sequence |
|---|---|
| AKAPIS | QIEYLAKQIVDNAIQQA (SEQ ID NO: 17) |
| AKAPIS-P | QIEYLAKQIPDNAIQQA (SEQ ID NO: 83) |
| Ht31 | KGADLIEEAASRIVDAVIEQVKAAG (SEQ ID NO: 84) |
| Ht31-P | KGADLIEEAASRIPDAPIEQVKAAG (SEQ ID NO: 85) |
| AKAP7δ-wt-pep | PEDAELVRLSKRLVENAVLKAVQQY (SEQ ID NO: 86) |
| AKAP7δ-L304T-pep | PEDAELVRTSKRLVENAVLKAVQQY (SEQ ID NO: 87) |
| AKAP7δ-L308D-pep | PEDAELVRLSKRDVENAVLKAVQQY (SEQ ID NO: 88) |
| AKAP7δ-P-pep | PEDAELVRLSKRLPENAVLKAVQQY (SEQ ID NO: 89) |
| AKAP7δ-PP-pep | PEDAELVRLSKRLPENAPLKAVQQY (SEQ ID NO: 90) |
| AKAP7δ-L314E-pep | PEDAELVRLSKRLVENAVEKAVQQY (SEQ ID NO: 91) |
| AKAP1-pep | EEGLDRNEEIKRAAFQIISQVISEA (SEQ ID NO: 92) |
| AKAP2-pep | LVDDPLEYQAGLLVQNAIQQAIAEQ (SEQ ID NO: 93) |
| AKAP5-pep | QYETLLIETASSLVKNAIQLSIEQL (SEQ ID NO: 94) |
| AKAP9-pep | LEKQYQEQLEEEVAKVIVSMSIAFA (SEQ ID NO: 95) |
| AKAP10-pep | NTDEAQEELAWKIAKMIVSDIMQQA (SEQ ID NO: 96) |
| AKAP11-pep | VNLDKKAVLAEKIVAEAIEKAEREL (SEQ ID NO: 97) |
| AKAP12-pep | NGILELETKSSKLVQNIIQTAVDQF (SEQ ID NO: 98) |
| AKAP14-pep | TQDKNYEDELTQVALALVEDVINYA (SEQ ID NO: 99) |
| Rab32-pep | ETSAKDNINIEEAARFLVEKILVNH (SEQ ID NO: 100) |

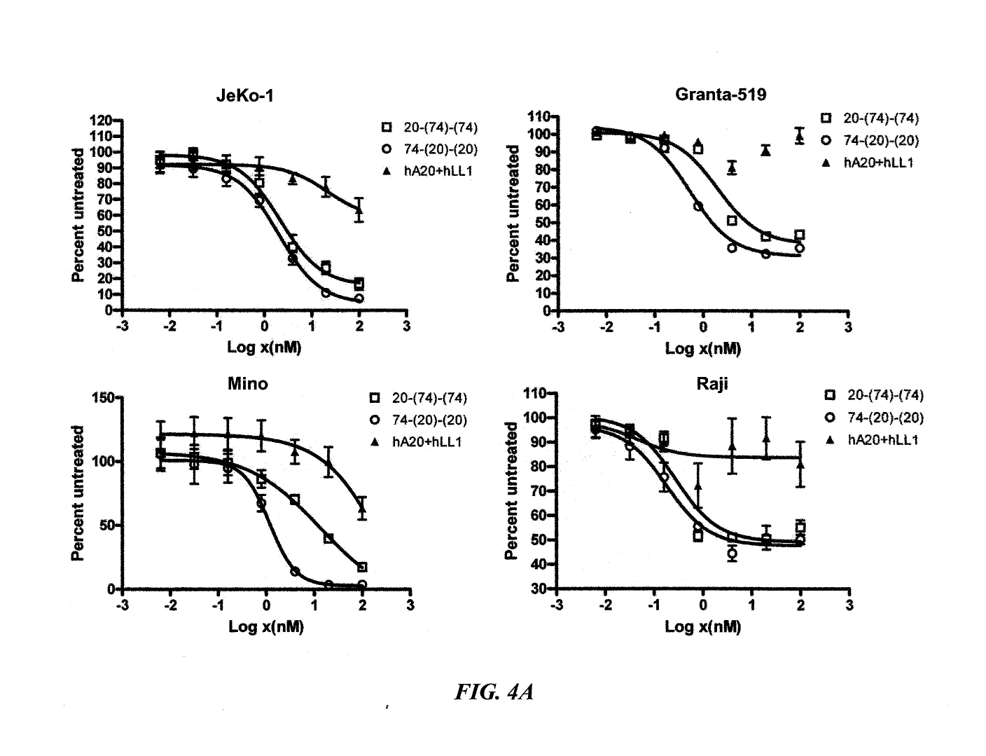

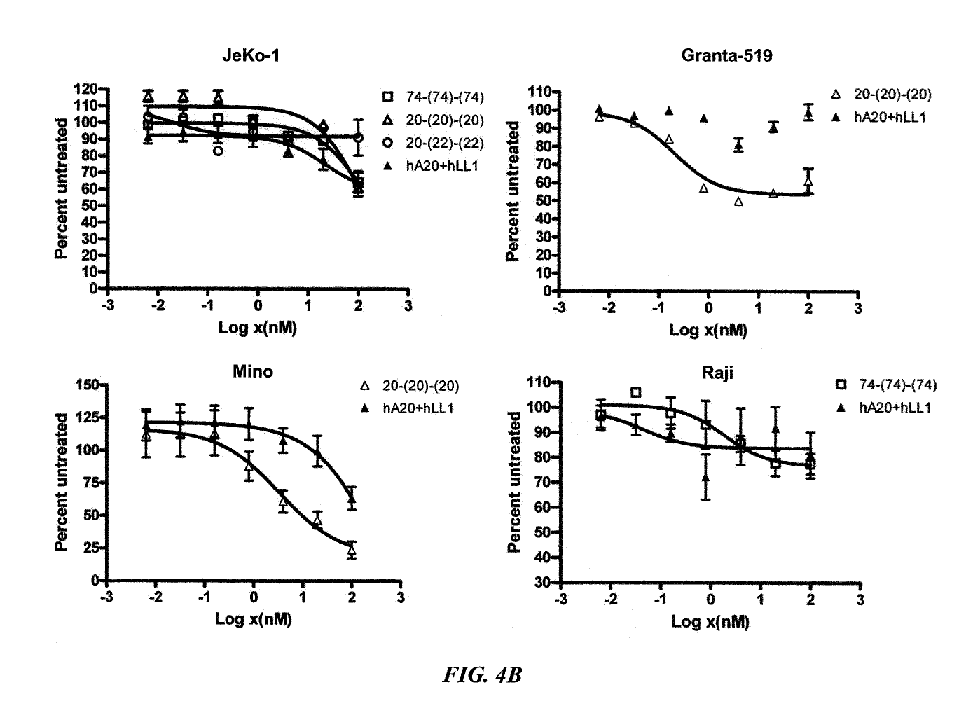

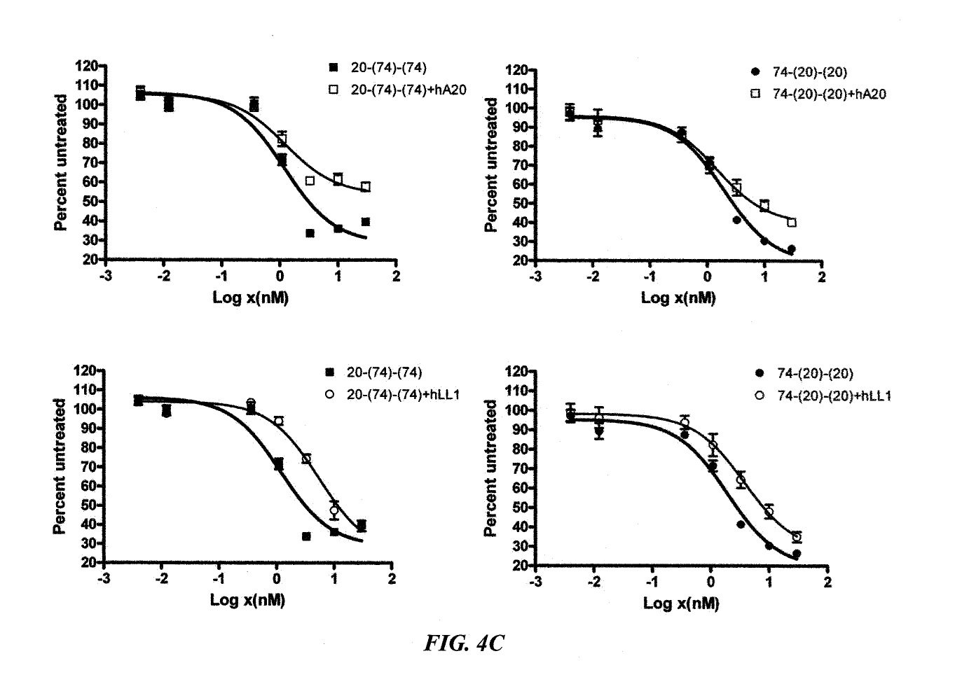

Residues that were highly conserved among the AD domains of different AKAP proteins are indicated below by underlining with reference to the AKAP IS sequence (SEQ ID NO:22). The residues are the same as observed by Alto et al. (2003), with the addition of the C-terminal alanine residue. (See FIG. 4 of Hundsrucker et al. (2006), incorporated herein by reference.) The sequences of peptide antagonists with particularly high affinities for the RII DDD sequence were those of AKAP-IS, AKAP7δ-wt-pep, AKAP7δ-L304T-pep and AKAP7δ-L308D-pep.

AKAP-IS

(SEQ ID NO: 17)

QIEYLAKQIVDNAIQQA

Can et al. (2001, J Biol Chem 276:17332-38) examined the degree of sequence homology between different AKAP-binding DDD sequences from human and non-human proteins and identified residues in the DDD sequences that appeared to be the most highly conserved among different DDD moieties. These are indicated below by underlining with reference to the human PKA RIIα DDD sequence of SEQ ID NO:15. Residues that were particularly conserved are further indicated by italics. The residues overlap with, but are not identical to those suggested by Kinderman et al. (2006) to be important for binding to AKAP proteins. The skilled artisan will realize that in designing sequence variants of DDD, it would be most preferred to avoid changing the most conserved residues (italicized), and it would be preferred to also avoid changing the conserved residues (underlined), while conservative amino acid substitutions may be considered for residues that are neither underlined nor italicized.

(SEQ ID NO: 15)

SHIQIPPGLTELLQGYTVEVLRQQPPDLVEFAVEYFTRLREARA

A modified set of conservative amino acid substitutions for the DDD1 (SEQ ID NO:15) sequence, based on the data of Carr et al. (2001) is shown in Table 5. The skilled artisan could readily derive alternative DDD amino acid sequences of use, as disclosed above for Table 2 and Table 3.

TABLE 5
Conservative Amino Acid Substitutions in DDD1
(SEQ ID NO: 15). Consensus sequence disclosed
as SEQ ID NO: 101.

| S | H | I | Q | [symbol] | [symbol] | P | [symbol] | [symbol] | T | E | [symbol] | [symbol] | Q | [symbol] | [symbol] | [symbol] | V | [symbol] | [symbol] | L | [symbol] |
|---|---|---|---|---|---|---|---|---|---|---|---|---|---|---|---|---|---|---|---|---|---|
| T | | | N | | | | | | S | | | | | | | | I | | | | |
| | | | | | | | | | | | | | | | | | L | | | | |
| | | | | | | | | | | | | | | | | | A | | | | |
| Q | [symbol] | [symbol] | P | [symbol] | [symbol] | V | E | [symbol] | [symbol] | V | E | [symbol] | [symbol] | T | R | [symbol] | R | E | A | [symbol] | A |
| N | | | | | | | | | | I | D | | | S | K | | K | | L | | L |
| | | | | | | | | | | L | | | | | | | | | I | | I |
| | | | | | | | | | | A | | | | | | | | | V | | V |

The skilled artisan will realize that these and other amino acid substitutions in the DDD or AD amino acid sequences may be utilized to produce alternative species within the genus of AD or DDD moieties, using techniques that are standard in the field and only routine experimentation.

Alternative DNL® Structures

In certain alternative embodiments, DNL® constructs may be formed using alternatively constructed antibodies or antibody fragments, in which an AD moiety may be attached at the C-terminal end of the kappa light chain (Cₖ), instead of the C-terminal end of the Fc on the heavy chain. The alternatively formed DNL® constructs may be prepared as disclosed in Provisional U.S. Patent Application Ser. Nos. 61/654,310, filed Jun. 1, 2012, 61/662,086, filed Jun. 20, 2012, 61/673,553, filed Jul. 19, 2012, and 61/682,531, filed Aug. 13, 2012, the entire text of each incorporated herein by reference. The light chain conjugated DNL® constructs exhibit enhanced Fc-effector function activity in vitro and improved pharmacokinetics, stability and anti-lymphoma activity in vivo (Rossi et al., 2013, Bioconjug Chem 24:63-71).

Cₖ-conjugated DNL® constructs may be prepared as disclosed in Provisional U.S. Patent Application Ser. Nos. 61/654,310, 61/662,086, 61/673,553, and 61/682,531. Briefly, Cₖ-AD2-IgG, was generated by recombinant engineering, whereby the AD2 peptide was fused to the C-terminal end of the kappa light chain. Because the natural C-terminus of Cₖ is a cysteine residue, which forms a disulfide bridge to C_{H}1, a 16-amino acid residue “hinge” linker was used to space the AD2 from the C_{K}-V_{H}1 disulfide bridge. The mammalian expression vectors for Cₖ-AD2-IgG-veltuzumab and Cₖ-AD2-IgG-epratuzumab were constructed using the pdHL2 vector, which was used previously for expression of the homologous C_{H}3-AD2-IgG modules. A 2208-bp nucleotide sequence was synthesized comprising the pdHL2 vector sequence ranging from the Bam HI restriction site within the V_{K}/C_{K} intron to the Xho I restriction site 3′ of the Cₖ intron, with the insertion of the coding sequence for the hinge linker (EFPKPSTPPGSSGGAP, SEQ ID NO:102) and AD2, in frame at the 3′end of the coding sequence for C_{K}. This synthetic sequence was inserted into the IgG-pdHL2 expression vectors for veltuzumab and epratuzumab via Bam HI and Xho I restriction sites. Generation of production clones with SpESFX-10 were performed as described for the C_{H}3-AD2-IgG modules. Cₖ-AD2-IgG-veltuzumab and Cₖ-AD2-IgG-epratuzumab were produced by stably-transfected production clones in batch roller bottle culture, and purified from the supernatant fluid in a single step using MabSelect (GE Healthcare) Protein A affinity chromatography.

Following the same DNL® process described previously for 22-(20)-(20) (Rossi et al., 2009, Blood 113:6161-71), Cₖ-AD2-IgG-epratuzumab was conjugated with C_{H}1-DDD2-Fab-veltuzumab, a Fab-based module derived from veltuzumab, to generate the bsHexAb 22*-(20)-(20), where the 22* indicates the Cₖ-AD2 module of epratuzumab and each (20) symbolizes a stabilized dimer of veltuzumab Fab. The properties of 22*-(20)-(20) were compared with those of 22-(20)-(20), the homologous Fc-bsHexAb comprising C_{H}3-AD2-IgG-epratuzumab, which has similar composition and molecular size, but a different architecture.

Following the same DNL® process described previously for 20-2b (Rossi et al., 2009, Blood 114:3864-71), Cₖ-AD2-IgG-veltuzumab, was conjugated with IFNα2b-DDD2, a module of IFNα2b with a DDD2 peptide fused at its C-terminal end, to generate 20*-2b, which comprises veltuzumab with a dimeric IFNα2b fused to each light chain. The properties of 20*-2b were compared with those of 20-2b, which is the homologous Fc-IgG-IFNα.

Each of the bsHexAbs and IgG-IFNα were isolated from the DNL® reaction mixture by MabSelect affinity chromatography. The two Cₖ-derived prototypes, an anti-CD22/CD20 bispecific hexavalent antibody, comprising epratuzumab (anti-CD22) and four Fabs of veltuzumab (anti-CD20), and a CD20-targeting immunocytokine, comprising veltuzumab and four molecules of interferon-a2b, displayed enhanced Fc-effector functions in vitro, as well as improved pharmacokinetics, stability and anti-lymphoma activity in vivo, compared to their Fc-derived counterparts.

Amino Acid Substitutions

In alternative embodiments, the disclosed methods and compositions may involve production and use of proteins or peptides with one or more substituted amino acid residues. For example, the DDD and/or AD sequences used to make DNL® constructs may be modified as discussed above.

The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.). Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Pre-Targeting

Bispecific or multispecific antibodies may be utilized in pre-targeting techniques. Pre-targeting is a multistep process originally developed to resolve the slow blood clearance of directly targeting antibodies, which contributes to undesirable toxicity to normal tissues such as bone marrow. With pre-targeting, a radionuclide or other therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Targetable Constructs

In certain embodiments, targetable construct peptides labeled with one or more therapeutic or diagnostic agents for use in pre-targeting may be selected to bind to a bispecific antibody with one or more binding sites for a targetable construct peptide and one or more binding sites for a target antigen associated with a disease or condition. Bispecific antibodies may be used in a pretargeting technique wherein the antibody may be administered first to a subject. Sufficient time may be allowed for the bispecific antibody to bind to a target antigen and for unbound antibody to clear from circulation. Then a targetable construct, such as a labeled peptide, may be administered to the subject and allowed to bind to the bispecific antibody and localize at the diseased cell or tissue.

Such targetable constructs can be of diverse structure and are selected not only for the availability of an antibody or fragment that binds with high affinity to the targetable construct, but also for rapid in vivo clearance when used within the pre-targeting method and bispecific antibodies (bsAb) or multispecific antibodies. Hydrophobic agents are best at eliciting strong immune responses, whereas hydrophilic agents are preferred for rapid in vivo clearance. Thus, a balance between hydrophobic and hydrophilic character is established. This may be accomplished, in part, by using hydrophilic chelating agents to offset the inherent hydrophobicity of many organic moieties. Also, sub-units of the targetable construct may be chosen which have opposite solution properties, for example, peptides, which contain amino acids, some of which are hydrophobic and some of which are hydrophilic.

Peptides having as few as two amino acid residues, preferably two to ten residues, may be used and may also be coupled to other moieties, such as chelating agents. The linker should be a low molecular weight conjugate, preferably having a molecular weight of less than 50,000 daltons, and advantageously less than about 20,000 daltons, 10,000 daltons or 5,000 daltons. More usually, the targetable construct peptide will have four or more residues, such as the peptide DOTA-Phe-Lys(HSG)-Tyr-Lys(HSG)-NH₂ (SEQ ID NO:103), wherein DOTA is 1,4,7,10-tetraazacyclododecane1,4,7,10-tetraacetic acid and HSG is the histamine succinyl glycyl group. Alternatively, DOTA may be replaced by NOTA (1,4,7-triaza-cyclononane-1,4,7-triacetic acid), TETA (p-bromoacetamido-benzyl-tetraethylaminetetraacetic acid), NETA ([2-(4,7-biscarboxymethyl[1,4,7]triazacyclononan-1-yl-ethyl]-2-carbonylmethyl-amino]acetic acid), NODA (1,4,7-triazacylononane-1,4-diacetate) or other known chelating moieties. Chelating moieties may be used, for example, to bind to a therapeutic and or diagnostic radionuclide, paramagnetic ion or contrast agent, such as Al-¹⁸F.

The targetable construct may also comprise unnatural amino acids, e.g., D-amino acids, in the backbone structure to increase the stability of the peptide in vivo. In alternative embodiments, other backbone structures such as those constructed from non-natural amino acids or peptoids may be used.

The peptides used as targetable constructs are conveniently synthesized on an automated peptide synthesizer using a solid-phase support and standard techniques of repetitive orthogonal deprotection and coupling. Free amino groups in the peptide, that are to be used later for conjugation of chelating moieties or other agents, are advantageously blocked with standard protecting groups such as a Boc group, while N-terminal residues may be acetylated to increase serum stability. Such protecting groups are well known to the skilled artisan. See Greene and Wuts Protective Groups in Organic Synthesis, 1999 (John Wiley and Sons, N.Y.). When the peptides are prepared for later use within the bispecific antibody system, they are advantageously cleaved from the resins to generate the corresponding C-terminal amides, in order to inhibit in vivo carboxypeptidase activity. Exemplary methods of peptide synthesis are disclosed in the Examples below.

Where pretargeting with bispecific antibodies is used, the antibody will contain a first binding site for an antigen produced by or associated with a target tissue and a second binding site for a hapten on the targetable construct. Exemplary haptens include, but are not limited to, HSG and In-DTPA. Antibodies raised to the HSG hapten are known (e.g. 679 antibody) and can be easily incorporated into the appropriate bispecific antibody (see, e.g., U.S. Pat. Nos. 6,962,702; 7,138,103 and 7,300,644, incorporated herein by reference with respect to the Examples sections). However, other haptens and antibodies that bind to them are known in the art and may be used, such as In-DTPA and the 734 antibody (e.g., U.S. Pat. No. 7,534,431, the Examples section incorporated herein by reference).

Preparation of Immunoconjugates

In preferred embodiments, a therapeutic or diagnostic agent may be covalently attached to an antibody or antibody fragment to form an immunoconjugate. Where the immunoconjugate is to be administered in concentrated form by subcutaneous, intramuscular or transdermal delivery, the skilled artisan will realize that only non-cytotoxic agents may be conjugated to the antibody. Where a second antibody or fragment thereof is administered by a different route, such as intravenously, either before, simultaneously with or after the subcutaneous, intramuscular or transdermal delivery, then the type of diagnostic or therapeutic agent that may be conjugated to the second antibody or fragment thereof is not so limited, and may comprise any diagnostic or therapeutic agent known in the art, including cytotoxic agents.

In some embodiments, a diagnostic and/or therapeutic agent may be attached to an antibody or fragment thereof via a carrier moiety. Carrier moieties may be attached, for example to reduced SH groups and/or to carbohydrate side chains. A carrier moiety can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the carrier moiety can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching carrier moieties to a targeting molecule involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.). For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.).

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.). Examples of such substituted cyclooctynes include difluorinated cyclooctynes, 4-dibenzocyclooctynol and azacyclooctyne (Id.). An alternative copper-free reaction involved strain-promoted alkyne-nitrone cycloaddition to give N-alkylated isoxazolines (Id.). The reaction was reported to have exceptionally fast reaction kinetics and was used in a one-pot three-step protocol for site-specific modification of peptides and proteins (Id.). Nitrones were prepared by the condensation of appropriate aldehydes with N-methylhydroxylamine and the cycloaddition reaction took place in a mixture of acetonitrile and water (Id.). These and other known click chemistry reactions may be used to attach carrier moieties to antibodies in vitro.

Agard et al. (2004, J Am Chem Soc 126:15046-47) demonstrated that a recombinant glycoprotein expressed in CHO cells in the presence of peracetylated N-azidoacetylmannosamine resulted in the bioincorporation of the corresponding N-azidoacetyl sialic acid in the carbohydrates of the glycoprotein. The azido-derivatized glycoprotein reacted specifically with a biotinylated cyclooctyne to form a biotinylated glycoprotein, while control glycoprotein without the azido moiety remained unlabeled (Id.). Laughlin et al. (2008, Science 320:664-667) used a similar technique to metabolically label cell-surface glycans in zebrafish embryos incubated with peracetylated N-azidoacetylgalactosamine. The azido-derivatized glycans reacted with difluorinated cyclooctyne (DIFO) reagents to allow visualization of glycans in vivo.

The Diels-Alder reaction has also been used for in vivo labeling of molecules. Rossin et al. (2010, Angew Chem Int Ed 49:3375-78) reported a 52% yield in vivo between a tumor-localized anti-TAG72 (CC49) antibody carrying a trans-cyclooctene (TCO) reactive moiety and an ¹¹¹In-labeled tetrazine DOTA derivative. The TCO-labeled CC49 antibody was administered to mice bearing colon cancer xenografts, followed 1 day later by injection of ¹¹¹In-labeled tetrazine probe (Id.). The reaction of radiolabeled probe with tumor localized antibody resulted in pronounced radioactivity localization in the tumor, as demonstrated by SPECT imaging of live mice three hours after injection of radiolabeled probe, with a tumor-to-muscle ratio of 13:1 (Id.). The results confirmed the in vivo chemical reaction of the TCO and tetrazine-labeled molecules.

Antibody labeling techniques using biological incorporation of labeling moieties are further disclosed in U.S. Pat. No. 6,953,675 (the Examples section of which is incorporated herein by reference). Such “landscaped” antibodies were prepared to have reactive ketone groups on glycosylated sites. The method involved expressing cells transfected with an expression vector encoding an antibody with one or more N-glycosylation sites in the CH1 or Vκ domain in culture medium comprising a ketone derivative of a saccharide or saccharide precursor. Ketone-derivatized saccharides or precursors included N-levulinoyl mannosamine and N-levulinoyl fucose. The landscaped antibodies were subsequently reacted with agents comprising a ketone-reactive moiety, such as hydrazide, hydrazine, hydroxylamino or thiosemicarbazide groups, to form a labeled targeting molecule. Exemplary agents attached to the landscaped antibodies included chelating agents like DTPA, large drug molecules such as doxorubicin-dextran, and acyl-hydrazide containing peptides. The landscaping technique is not limited to producing antibodies comprising ketone moieties, but may be used instead to introduce a click chemistry reactive group, such as a nitrone, an azide or a cyclooctyne, onto an antibody or other biological molecule.

Modifications of click chemistry reactions are suitable for use in vitro or in vivo. Reactive targeting molecule may be formed either by either chemical conjugation or by biological incorporation. The targeting molecule, such as an antibody or antibody fragment, may be activated with an azido moiety, a substituted cyclooctyne or alkyne group, or a nitrone moiety. Where the targeting molecule comprises an azido or nitrone group, the corresponding targetable construct will comprise a substituted cyclooctyne or alkyne group, and vice versa. Such activated molecules may be made by metabolic incorporation in living cells, as discussed above.

Alternatively, methods of chemical conjugation of such moieties to biomolecules are well known in the art, and any such known method may be utilized. General methods of immunoconjugate formation are disclosed, for example, in U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.

Therapeutic and Diagnostic Agents

In certain embodiments, the antibodies or fragments thereof may be used in combination with one or more therapeutic and/or diagnostic agents. Where the agent is attached to an antibody or fragment thereof to be administered by subcutaneous, intramuscular or transdermal administration, then only non-cytotoxic agents are contemplated. Non-cytotoxic agents may include, without limitation, immunomodulators, cytokines (and their inhibitors), chemokines (and their inhibitors), tyrosine kinase inhibitors, growth factors, hormones and certain enzymes (i.e., those that do not induce local necrosis), or their inhibitors. Where the agent is co-administered either before, simultaneously with or after the subcutaneous, intramuscular or transdermal antibody formulation, then cytotoxic agents may be utilized. An agent may be administered as an immunoconjugate with a second antibody or fragment thereof, or may be administered as a free agent. The following discussion applies to both cytotoxic and non-cytotoxic agents.

Therapeutic agents may be selected from the group consisting of a radionuclide, an immunomodulator, an anti-angiogenic agent, a cytokine, a chemokine, a growth factor, a hormone, a drug, a prodrug, an enzyme, an oligonucleotide, a pro-apoptotic agent, an interference RNA, a photoactive therapeutic agent, a tyrosine kinase inhibitor, a Bruton kinase inhibitor, a sphingosine inhibitor, a cytotoxic agent, which may be a chemotherapeutic agent or a toxin, and a combination thereof. The drugs of use may possess a pharmaceutical property selected from the group consisting of antimitotic, antikinase, alkylating, antimetabolite, antibiotic, alkaloid, anti-angiogenic, pro-apoptotic agents, and combinations thereof.

Exemplary drugs may include, but are not limited to, 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, paclitaxel, temazolomide (an aqueous form of DTIC), transplatinum, thalidomide, thioguanine, thiotepa, teniposide, topotecan, uracil mustard, vinorelbine, vinblastine, vincristine and vinca alkaloids.

Toxins may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Immunomodulators may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β, -λ, or -γ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, -λ and -γ; colony stimulating factors (CSFs) such as macrophage-CSF (M-CSF); interleukins (ILs) such as IL-1, IL-la, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-23, IL-25, LIF, kit-ligand or FLT-3, angiostatin, thrombospondin, endostatin, tumor necrosis factor and lymphotoxin.

Chemokines of use include RANTES, MCAF, MIP1-alpha, MIP1-Beta and IP-10.

Radioactive isotopes include, but are not limited to—¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ²²⁷Th, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ⁷⁷Br, ¹¹³In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ²²⁷Th, ⁷⁶Br, ¹⁶⁹Yb, and the like.

A variety of tyrosine kinase inhibitors are known in the art and any such known therapeutic agent may be utilized. Exemplary tyrosine kinase inhibitors include, but are not limited to canertinib, dasatinib, erlotinib, gefitinib, imatinib, lapatinib, leflunomide, nilotinib, pazopanib, semaxinib, sorafenib, sunitinib, sutent and vatalanib. A specific class of tyrosine kinase inhibitor is the Bruton tyrosine kinase inhibitor. Bruton tyrosine kinase (Btk) has a well-defined role in B-cell development. Bruton kinase inhibitors include, but are not limited to, PCI-32765 (ibrutinib), PCI-45292, GDC-0834, LFM-A13 and RN486.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni et al. (eds.), PHOTODYNAMIC THERAPY OF TUMORS AND OTHER DISEASES (Libreria Progetto 1985); van den Bergh, Chem. Britain (1986), 22:430. Moreover, monoclonal antibodies have been coupled with photoactivated dyes for achieving phototherapy. See Mew et al., J. Immunol. (1983),130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Corticosteroid hormones can increase the effectiveness of other chemotherapy agents, and consequently, they are frequently used in combination treatments. Prednisone and dexamethasone are examples of corticosteroid hormones.

In certain embodiments, anti-angiogenic agents, such as angiostatin, baculostatin, canstatin, maspin, anti-placenta growth factor (P1GF) peptides and antibodies, anti-vascular growth factor antibodies (such as anti-VEGF and anti-P1GF), anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, Gro-β, thrombospondin, 2-methoxyoestradiol, proliferin-related protein, carboxiamidotriazole, CM101, Marimastat, pentosan polysulphate, angiopoietin-2, interferon-alpha, interferon-lambda, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline may be of use.

The therapeutic agent may comprise an oligonucleotide, such as a siRNA. The skilled artisan will realize that any siRNA or interference RNA species may be attached to an antibody or fragment thereof for delivery to a targeted tissue. Many siRNA species against a wide variety of targets are known in the art, and any such known siRNA may be utilized in the claimed methods and compositions.

Known siRNA species of potential use include those specific for IKK-gamma (U.S. Pat. No. 7,022,828); VEGF, Flt-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (7,781,575), and apolipoprotein B (7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Minis Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL complexes.

Exemplary siRNA species known in the art are listed in Table 6. Although siRNA is delivered as a double-stranded molecule, for simplicity only the sense strand sequences are shown in Table 6.

TABLE 6
Exemplary siRNA Sequences

| Target | Sequence | SEQ ID NO |
|---|---|---|
| VEGF R2 | AATGCGGCGGTGGTGACAGTA | SEQ ID NO: 104 |
| VEGF R2 | AAGCTCAGCACACAGAAAGAC | SEQ ID NO: 105 |
| CXCR4 | UAAAAUCUUCCUGCCCACCdTdT | SEQ ID NO: 106 |
| CXCR4 | GGAAGCUGUUGGCUGAAAAdTdT | SEQ ID NO: 107 |
| PPARC1 | AAGACCAGCCUCUUUGCCCAG | SEQ ID NO: 108 |
| Dynamin 2 | GGACCAGGCAGAAAACGAG | SEQ ID NO: 109 |
| Catenin | CUAUCAGGAUGACGCGG | SEQ ID NO: 110 |
| E1A binding protein | UGACACAGGCAGGCUUGACUU | SEQ ID NO: 111 |
| Plasminogen | GGTGAAGAAGGGCGTCCAA | SEQ ID NO: 112 |
| activator | | |
| K-ras | GATCCGTTGGAGCTGTTGGCGTAGTT | SEQ ID NO: 113 |
| | CAAGAGACTCGCCAACAGCTCCAACT | |
| | TTTGGAAA | |
| Sortilin 1 | AGGTGGTGTTAACAGCAGAG | SEQ ID NO: 114 |
| Apolipoprotein E | AAGGTGGAGCAAGCGGTGGAG | SEQ ID NO: 115 |
| Apolipoprotein E | AAGGAGTTGAAGGCCGACAAA | SEQ ID NO: 116 |
| Bcl-X | UAUGGAGCUGCAGAGGAUGdTdT | SEQ ID NO: 117 |
| Raf-1 | TTTGAATATCTGTGCTGAGAACACA | SEQ ID NO: 118 |
| | GTTCTCAGCACAGATATTCTTTTT | |
| Heat shock | AATGAGAAAAGCAAAAGGTGCCCTGTCTC | SEQ ID NO: 119 |
| transcription factor 2 | | |
| IGFBP3 | AAUCAUCAUCAAGAAAGGGCA | SEQ ID NO: 120 |
| Thioredoxin | AUGACUGUCAGGAUGUUGCdTdT | SEQ ID NO: 121 |
| CD44 | GAACGAAUCCUGAAGACAUCU | SEQ ID NO: 122 |
| MMP14 | AAGCCTGGCTACAGCAATATGCCTGTCTC | SEQ ID NO: 123 |
| MAPKAPK2 | UGACCAUCACCGAGUUUAUdTdT | SEQ ID NO: 124 |
| FGFR1 | AAGTCGGACGCAACAGAGAAA | SEQ ID NO: 125 |
| ERBB2 | CUACCUUUCUACGGACGUGdTdT | SEQ ID NO: 126 |
| BCL2L1 | CTGCCTAAGGCGGATTTGAAT | SEQ ID NO: 127 |
| ABL1 | TTAUUCCUUCUUCGGGAAGUC | SEQ ID NO: 128 |
| CEACAM1 | AACCTTCTGGAACCCGCCCAC | SEQ ID NO: 129 |
| CD9 | GAGCATCTTCGAGCAAGAA | SEQ ID NO: 130 |
| CD151 | CATGTGGCACCGTTTGCCT | SEQ ID NO: 131 |
| Caspase 8 | AACTACCAGAAAGGTATACCT | SEQ ID NO: 132 |
| BRCA1 | UCACAGUGUCCUUUAUGUAdTdT | SEQ ID NO: 133 |
| p53 | GCAUGAACCGGAGGCCCAUTT | SEQ ID NO: 134 |
| CEACAM6 | CCGGACAGTTCCATGTATA | SEQ ID NO: 135 |

The skilled artisan will realize that Table 6 represents a very small sampling of the total number of siRNA species known in the art, and that any such known siRNA may be utilized in the claimed methods and compositions.

Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹⁸F, ⁵²Fe, ¹¹⁰In, ¹¹¹In, ¹⁷⁷Lu, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²Mn, ⁵⁵Co, ⁷²As, ⁷⁵Br, ⁷⁶Br, ₈₂ₘRb, ⁸³⁵r, or other gamma-, beta-, or positron-emitters.

Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III).

Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Methods of Administration

The subject antibodies and immunoglobulins in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active ingredients (i.e., the labeled molecules) are combined in a mixture with one or more pharmaceutically suitable excipients. Sterile phosphate-buffered saline is one example of a pharmaceutically suitable excipient. Other suitable excipients are well known to those in the art. See, e.g., Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The preferred route for administration of the compositions described herein is parenteral injection, more preferably by subcutaneous, intramuscular or transdermal delivery. Other forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives.

Formulated compositions comprising antibodies can be used for subcutaneous, intramuscular or transdermal administration. Compositions can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The compositions may be administered in solution. The formulation thereof should be in a solution having a suitable pharmaceutically acceptable buffer such as phosphate, TRIS (hydroxymethyl) aminomethane-HCl or citrate and the like. Buffer concentrations should be in the range of 1 to 100 mM. The formulated solution may also contain a salt, such as sodium chloride or potassium chloride in a concentration of 50 to 150 mM. An effective amount of a stabilizing agent such as mannitol, trehalose, sorbitol, glycerol, albumin, a globulin, a detergent, a gelatin, a protamine or a salt of protamine may also be included.

The dosage of an administered antibody for humans will vary depending upon such factors as the patient's age, weight, height, sex, general medical condition and previous medical history. Typically, it is desirable to provide the recipient with a dosage of antibody that is in the range of from about 1 mg to 600 mg as a single infusion or single or multiple injections, although a lower or higher dosage also may be administered. Typically, it is desirable to provide the recipient with a dosage that is in the range of from about 50 mg per square meter (m²) of body surface area or 70 to 85 mg of the antibody for the typical adult, although a lower or higher dosage also may be administered. Examples of dosages of antibodies that may be administered to a human subject are 1 to 1,000 mg, more preferably 1 to 70 mg, most preferably 1 to 20 mg, although higher or lower doses may be used. Dosages may be repeated as needed, for example, once per week for 4-10 weeks, preferably once per week for 8 weeks, and more preferably, once per week for 4 weeks. It may also be given less frequently, such as every other week for several months, or more frequently, such as twice weekly or by continuous infusion.

More recently, subcutaneous administration of veltuzumab has been given to NHL patients in 4 doses of 80, 160 or 320 mg, repeated every two weeks (Negrea et al., 2011, Haematologica 96:567-73). Only occasional, mild to moderate and transient injection reactions were observed, with no other safety issues (Id.). The objective response rate (CR+CRu+PR) was 47%, with a CR/CRu (complete response) rate of 24% (Id.). Interestingly, the 80 mg dosage group showed the highest percentage of objective response (2/3, 67%), with one of three patients showing a complete response (Id.). Four out of eight objective responses continued for 60 weeks (Id.). All serum samples evaluated for HAHA were negative (Id.). Although the low sample population reported in this study precludes any definitive conclusions on optimal dosing, it is apparent that therapeutic response was observed at the lowest dosage tested (80 mg).

In certain alternative embodiments, the antibody may be administered by transdermal delivery. Different methods of transdermal delivery are known in the art, such as by transdermal patches or by microneedle devices, and any such known method may be utilized. In an exemplary embodiment, transdermal delivery may utilize a delivery device such as the 3M hollow Microstructured Transdermal System (hMTS) for antibody based therapeutics. The hMTS device comprises a 1 cm² microneedle array consisting of 18 hollow microneedles that are 950 microns in length, which penetrate approximately 600-700 microns into the dermal layer of the skin where there is a high density of lymphatic channels. A spring-loaded device forces the antibody composition from a fluid reservoir through the microneedles for delivery to the subject. Only transient erythema and edema at the injection site are observed (Burton et al., 2011, Pharm Res 28:31-40). The hMTS device is not perceived as a needle injector, resulting in improved patient compliance.

In alternative embodiments, transdermal delivery of peptides and proteins may be achieved by (1) coadministering with a synthetic peptide comprising the amino acid sequence of ACSSSPSKHCG (SEQ ID NO:136) as reported by Chen et al. (Nat Biotechnol 2006; 24: 455-460) and Carmichael et al. (Pain 2010; 149:316-324); (2) coadministering with arginine-rich intracellular delivery peptides as reported by Wang et al. (BBRC 2006; 346: 758-767); (3) coadminstering with either AT1002 (FCIGRLCG, SEQ ID NO:137) or Tat (GRKKRRNRRRCG, SEQ ID NO:138) as reported by Uchida et al. (Chem Pharm Bull 2011; 59:196); or (4) using an adhesive transdermal patch as reported by Jurynczyk et al (Ann Neurol 2010; 68:593-601). In addition, transdermal delivery of negatively charged drugs may be facilitated by combining with the positively charged, pore-forming magainin peptide as reported by Kim et al. (Int J Pharm 2008; 362:20-28).

In preferred embodiments where the antibody is administered subcutaneously, intramuscularly or transdermally in a concentrated formulation, the volume of administration is preferably limited to 3 ml or less, more preferably 2 ml or less, more preferably 1 ml or less. The use of concentrated antibody formulations allowing low volume subcutaneous, intramuscular or transdermal administration is preferred to the use of more dilute antibody formulations that require specialized devices and ingredients (e.g., hyaluronidase) for subcutaneous administration of larger volumes of fluid, such as 10 ml or more. The subcutaneous, intramuscular or transdermal delivery may be administered as a single administration to one skin site or alternatively may be repeated one or more times, or even given to more than one skin site in one therapeutic dosing session. However, the more concentrated the formulation, the lower the volume injected and the fewer injections will be needed for each therapeutic dosing.

Methods of Use

In preferred embodiments, the anti-CD74/CD20 antibodies or fragments thereof are of use for therapy of B-cell malignancies, such as NHL. Examples of cancers include, but are not limited to, lymphoma, leukemia and lymphoid malignancies. In preferred embodiments, the antibodies or fragments thereof are of use to treat hematopoietic cancers. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: acute childhood lymphoblastic leukemia, acute lymphoblastic leukemia, acute lymphocytic leukemia, acute myeloid leukemia, adult acute lymphocytic leukemia, adult acute myeloid leukemia, adult Hodgkin's disease, adult Hodgkin's lymphoma, adult lymphocytic leukemia, adult non-Hodgkin's lymphoma, AIDS-related lymphoma, AIDS-related malignancies, central nervous system (primary) lymphoma, central nervous system lymphoma, childhood acute lymphoblastic leukemia, childhood acute myeloid leukemia, childhood Hodgkin's disease, childhood Hodgkin's lymphoma, childhood lymphoblastic leukemia, childhood non-Hodgkin's lymphoma, chronic lymphocytic leukemia, chronic myelogenous leukemia, cutaneous T-cell lymphoma, hairy cell leukemia, Hodgkin's disease, Hodgkin's lymphoma, hypergammaglobulinemia, lymphoproliferative disorders, macroglobulinemia, multiple myeloma, multiple myeloma/plasma cell neoplasm, myelodysplastic syndrome, myelogenous leukemia, myeloid leukemia, myeloproliferative disorders, non-Hodgkin's lymphoma during pregnancy, plasma cell neoplasm/multiple myeloma, primary central nervous system lymphoma, T-cell lymphoma, Waldenstrom's macroglobulinemia, and any other hyperproliferative disease.

The methods and compositions described and claimed herein may be used to detect or treat malignant or premalignant conditions. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)).

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, and Waldenstrom's macroglobulinemia.

Exemplary autoimmune diseases include acute idiopathic thrombocytopenic purpura, chronic immune thrombocytopenia, dermatomyositis, Sydenham's chorea, myasthenia gravis, systemic lupus erythematosus, lupus nephritis, rheumatic fever, polyglandular syndromes, bullous pemphigoid, pemphigus vulgaris, juvenile diabetes mellitus, Henoch-Schonlein purpura, post-streptococcal nephritis, erythema nodosum, Takayasu's arteritis, ANCA-associated vasculitides, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjögren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis and fibrosing alveolitis.

Kits

Various embodiments may concern kits containing components suitable for treating diseased tissue in a patient. Exemplary kits may contain at least one anti-CD20 antibody or fragment thereof and at least one anti-CD74 antibody or fragment thereof, as described herein. A device capable of delivering the kit components by injection, for example, a syringe for subcutaneous injection, may be included. Where transdermal administration is used, a delivery device such as hollow microneedle delivery device may be included in the kit. Exemplary transdermal delivery devices are known in the art, such as 3M's hollow Microstructured Transdermal System (hMTS), and any such known device may be used.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Alternatively, the antibody or fragment may be delivered and stored as a liquid formulation. Other containers that may be used include, but are not limited to, a pouch, tray, box, tube, or the like. Kit components may be packaged and maintained sterilely within the containers. Another component that can be included is instructions to a person using a kit for its use.

### EXAMPLES

Various embodiments of the present invention are illustrated by the following examples, without limiting the scope thereof.

### Example 1

### Combination Therapy of Relapsed/Refractory NHL with Anti-CD74 and Anti-CD20 Antibodies

Preclinical studies demonstrated the efficacy of milatuzumab, a humanized anti-CD74 antibody combined with anti-CD20 monoclonal antibodies in vitro and in an in vivo preclinical model of mantle cell lymphoma (Alinari et al., 2009, Blood 114, Abstract 1694; Alinari et al., 2011, Blood 117:4530-41; Gupta et al., 2012, Blood 119:3767-78). As a result of the anti-tumor activity observed in vitro and in vivo with combined anti-CD20 and anti-CD74 antibodies, we initiated a phase I/II trial of veltuzumab and milatuzumab in patients with relapsed or refractory B-cell non-Hodgkin's lymphoma after at least one prior therapy to determine the safety, tolerability, and overall response rate with this combination. Methods: Patients received an induction consisting of veltuzumab at 200 mg/m² on day 1 of weeks 1-4 combined with escalating doses of milatuzumab at 8, 16, and 20 mg/kg on day 2 of week 1 and days 1 and 4 of weeks 2-4. Patients without disease progression could continue to receive an extended induction consisting of veltuzumab 200 mg/m² on day 1 with milatuzumab at the same doses as during induction on days 1 and 4 of weeks 12, 20, 28, and 36. Dose limiting toxicity (DLT) was defined during weeks 1-4. Although not defined as a DLT, 3 of the first 6 patients enrolled at dose levels 1-2, had grade 3 infusion reactions with milatuzumab. The study was amended to drop milatuzumab dosing on day 1 of weeks 2-4, 12, 20, 28, and 36 and to add 20 mg dexamethasone immediately prior to and 10 mg post each dose of milatuzumab. Enrollment resumed with 3 additional patients at dose levels 1 and 2. Dose escalation reached dose level 3 and was followed by a phase II study which enrolled a total of 17 patients. Results: A total of 35 patients enrolled on the study with follicular lymphoma (46%), diffuse large B-cell lymphoma (23%), mantle cell lymphoma (17%), lymphoplasmacytic lymphoma (9%), and marginal zone lymphoma (6%). Median age was 63 (range 39-82), median number of prior therapies was 3 (range 1-10), and 63% of patients were rituximab refractory. No dose limiting toxicities were observed in the phase I study. Related grade 3-4 toxicities included lymphopenia (31%), leukopenia (9%), neutropenia (11%), anemia (3%), infusion reactions (14%), hyperglycemia (6%), fatigue (3%), and atrial tachycardia (3%). Median weeks of therapy received was 12 and 29% of patients completed all 36 weeks of therapy. The overall response rate was 24%, median duration of response was 12 months, and responses were observed at all dose levels. Responses were observed in patients with FL, MZL, and MCL, and 50% of responding patients were refractory to rituximab. Conclusions: Combination therapy with veltuzumab and milatuzumab was well-tolerated and demonstrated activity in a population of heavily pre-treated patients with relapsed or refractory indolent non-Hodgkin's lymphoma.

Introduction

With the use of rituximab in the treatment of B-cell non-Hodgkin's lymphoma (NHL), as a single agent (Coiffier et al., 1998, Blood 92:1927-32; McLaughlin et al., 1998, J Clin Oncol 16:2825-33; Hainsworth et al., 2000, Blood 95:3052-56), in combination with chemotherapy (Czuczman et al., 1999, J Clin Oncol 17:268-76; Coiffier et al., 2002, N Engl J Med 346:235-42), and in maintenance therapy (e.g., van Oers et al., 2006, Blood 108:3295-301), novel approaches are increasingly important to overcome rituximab resistance for patients with relapsed disease. Milatuzumab is a humanized anti-CD74 monoclonal antibody which has shown activity against several NHL cell lines in vitro. CD74 is a transmembrane protein which associates with MCH class II α and β chains and is expressed on normal monocytes, macrophages, dendritic cells, and malignant B-cells, including 90% of B-cell NHL, chronic lymphocytic leukemia, and multiple myeloma specimens (Stein et al., 2007, Clin Cancer Res 13:5556s-63s). CD74 serves as a chaperone for MHC class II molecules, functions in B-cell survival pathways, and activates syk, phophatidylinositol 3-kinase, AKT, and nuclear factor (NF)-κB with resultant transcription of anti-apoptotic genes including bcl-xl (Stein et al., 2007, Clin Cancer Res 13:5556s-63s). Although CD74 is observed on normal B-cells, monocytes, macrophages, and dendritic cells, milatuzumab appears to selectively target malignant B-cells, with minimal impact on the viability of normal NK, T, dendritic, or B-cells (Chen et al., 2009, ASH Annual Meeting Abstracts, 114:2744 (Abstr); Hertlein et al., 2009, ASH Annual Meeting Abstracts, 114:721 (Abstr). In vivo studies confirm the single agent activity of milatuzumab, with a prolongation of survival observed in murine Raji and Daudi xenograft models following milatuzumab therapy (Stein et al., 2007, Clin Cancer Res 13:5556s-63s). A phase I study of milatuzumab in multiple myeloma utilizing doses of 1.5, 4, 8 and 16 mg/kg demonstrated no objective responses but was well-tolerated. One grade 3 infusion reaction occurred, but the remaining infusion reactions were grade 1-2. The incidence of anemia was 28% and other hematologic toxicities were not observed (Kaufman et al., 2013, Br J Haematol 163:478-86).

Veltuzumab is a fully humanized anti-CD20 antibody. Elimination of the murine components of the antibody was postulated to favorably alter pharmacokinetics and toxicity profiles through the reduction of human anti-chimeric antibody responses and an increase in the serum T1/2 to permit extended dosing intervals, limit infusion reactions, allow use of smaller doses, and ultimately improve efficacy (Stein et al., 2004, Clin Cancer Res 10:2868-78). In vitro, veltuzumab appears similar to rituximab with similar antigen-binding specificity, binding avidity, and dissociation. Induction of apoptosis, ADCC, and complement mediated cytotoxicity (CDC) appear similar between veltuzumab and rituximab (Stein et al., 2004, Clin Cancer Res 10:2868-78). However, in vivo in 3 different NHL SCID mouse models, veltuzumab significantly improves survival compared to rituximab with repetitive dosing (Goldenberg et al., 2009, Blood 113:1062-70). This improved efficacy may be due in part to a single amino acid change in the complementarity determining region, CDR3-VH, of veltuzumab compared to rituximab that contributes to a lower off-rate of veltuzumab in pre-clinical models (Goldenberg et al., 2009, Blood 113:1062-70). In a phase VII trial, 82 patients received 4 weekly doses of veltuzumab ranging from 80-750 mg/m². Infusion times were 60-120 minutes and toxicities consisted primarily of grade 1-2 infusion reactions, with no grade 3-4 adverse events. The ORR was 40.7%, with 21% complete responses. Responses were observed in patients previously treated with 2-5 prior rituximab-containing regimens and in patients with follicular, marginal zone, diffuse large cell, and MCL at all dose levels (Morschhauser et al., 2009, J Clin Oncol 27:3346-53). Subcutaneous administration of veltuzumab was evaluated in a phase I study, where patients received 80, 160, or 320 mg every other week for a total of 4 doses. For the seventeen patients enrolled in the study with predominantly follicular histology, the ORR was 47%.

A preclinical analysis demonstrated that combined treatment with rituximab and milatuzumab led to enhanced cell death in NHL cell lines through actin polymerization at the cell surface, loss of NF-κB survival signals, generation of radical oxygen species, and loss of mitochondrial membrane potential (Alinari et al., 2011, Blood 117:4530-41). Therefore, based on these promising preclinical data, we conducted a phase I/II trial of veltuzumab and milatuzumab in patients with relapsed and refractory NHL. The fully humanized anti-CD20 antibody veltuzumab was utilized instead of rituximab due to the lower potential risk of infusion reactions with the humanized antibody, greater single efficacy with veltuzumab compared to rituximab in a NHL SCID mouse model (Goldenberg et al., 2009, Blood 113:1062-70), and encouraging clinical efficacy with single agent veltuzumab in patients with a variety of NHL subtypes who have previously received rituximab (Morschhauser et al., J Clin Oncol 27:3346-53; Alinari et al., 2011, Blood 118:6893-903), as most enrolling patients were anticipated to be heavily pretreated with rituximab. Advantages of dual monoclonal antibody therapy include favorable toxicity profiles which permit frequent dosing and maintenance treatment, particularly in heavily pre-treated or older patients; potentially increased efficacy when compared to single agent regimens; and the ability to overcome resistance mechanisms that develop in the setting of single agent therapy.

Patients and Methods

Patient Selection—

The study opened to accrual on Jan. 22, 2010 and closed to accrual on May 31, 2013. Inclusion criteria included patients with histologically confirmed B-cell NHL including marginal zone lymphoma, Waldenstrom's macroglobulinemia or lymphoplasmacytic lymphoma, follicular lymphoma, diffuse large B-cell lymphoma, transformed lymphoma, and mantle cell lymphoma by the World Health Organization criteria. Patients were required to have relapsed or refractory disease after at least one prior therapy. Prior rituximab was permitted under the following conditions: patients who were rituximab-refractory, defined as having less than a partial response to the previous rituximab-containing regimen were eligible at any time, and patients who were rituximab-sensitive, defined as having a complete response or partial response to the last rituximab-containing regimen, were eligible at least three months after the last infusion of rituximab. Additional inclusion criteria were age >18 years, Eastern Cooperative Oncology Group (ECOG) performance status ≦2, an absolute neutrophil count ≧1000/ μL, platelets ≧75,000/μL, total bilirubin ≦2.0 times the institutional upper limit of normal, AST(SGOT)/ALT(SGPT)≦2.5 times the institutional upper limit of normal, and creatinine ≦2.0 mg/dL. Patients who had relapsed after stem cell transplant were eligible for this trial. Patients with diffuse large B-cell lymphoma were eligible provided they had undergone or were not candidates for autologous stem cell transplant. Exclusion criteria included HIV infection, hepatitis B, central nervous system involvement with lymphoma, a history of human anti-globulin antibodies, active secondary malignancies, and pregnant or nursing women. In the phase II portion of the study, measurable disease was required. The institutional review board of Ohio State University approved the protocol, and all patients provided written informed consent. The Ohio State University Data Safety Monitoring Board provided oversight and safety monitoring for the study. The trial was registered at clinicaltrials.gov (NCT00989586).

Study Design—

The phase I study was conducted utilizing a standard cohorts of 3 dose escalation schema with three to six patients treated at each dose level to determine the dose limiting toxicity (DLT) and maximum tolerated dose (MTD) of the combination. The MTD was defined as that dose beneath the dose at which 2 or more of 6 patients experienced DLT. A phase II study designed to determine the overall response rate for the combination followed the phase I study at the recommended phase II dose.

Patients received veltuzumab at 200 mg/m² weekly combined with escalating doses of milatuzumab at 8, 16, and 20 mg/kg for four weeks of induction therapy. During week 1 of induction therapy, patients received veltuzumab alone on day 1 and milatuzumab alone on day 2 to prevent overlapping infusion reactions. Starting in week 2, veltuzumab was given first on day 1 followed by milatuzumab on days 1 and 4. Patients without progressive disease or unacceptable toxicity following induction therapy were eligible to receive extended induction, which consisted of veltuzumab on day 1 and milatuzumab on days 1 and 4 of weeks 12, 20, 28, and 36. All patients received premedication with acetaminophen, diphenhydramine, hydrocortisone 50 mg, and famotidine 20 mg prior to veltuzumab and milatuzumab doses. The actual body weight was used for all dose calculations for all cycles. No dose reductions of veltuzumab or milatuzumab were permitted, although delays for toxicity were permitted. DLT was defined during weeks 1 through 4. Dose limiting toxicity was defined as any of the following: treatment delays >14 days, any grade 3-4 non-hematologic toxicity with the exception of infusion reactions, grade 4 febrile neutropenia, grade 3 febrile neutropenia or infection with fever or infection that failed to resolve within 7 days, and grade 4 neutropenia or thrombocytopenia persisting >7 days.

Assessment of Toxicity and Response—

Toxicity was graded according to NCI Common Toxicity Criteria for Adverse Events v. 3.0 and classified as either unrelated, unlikely, possibly, probably, or definitely related to study treatment. Response was assessed by CT scans during weeks 5, 10, 24, 40 and then every 4 months until disease progression or for a maximum 2 years. Combined PET/CT was performed at weeks 5 and 40. Patients removed from study for unacceptable adverse events were followed until resolution of any treatment-related abnormalities or changes that warranted additional follow-up.

Pharmacokinetics (PK)—

Blood samples for veltuzumab PK were collected at the following time points: pre- and post-infusion on day 1 of weeks 1, 2, 4, 12 and 36. One additional veltuzumab blood sample was also collected each of weeks 5 through 10 (post 4^{th} infusion). Blood samples for milatuzumab PK were collected at the following time points: pre- and post-infusion on day 2 of week 1 and day 1 of weeks 2, 4, 12 and 36. Additional samples were collected days 3 through 6 of week 1 (post 1^{st} infusion). Serum veltuzumab and milatuzumab levels were determined by using enzyme-linked immunosorbent assay (ELISA) performed by the Study Sponsor. Concentration-time data were analyzed using WinNonlin 6.3 (Pharsight, Mountain View, Calif.). Standard non-compartmental analyses (NCA) were performed with concentration-time data from the 1⁴ infusion of milatuzumab (20 mg/kg cohort) and 4^{th} dose of veltuzumab. NCA was unable to be performed for the 8 mg/kg and 16 mg/kg milatuzumab cohorts due to sparse data points in the terminal phase. Veltuzumab data was also analyzed using one-compartment PK model.

Immunogenicity—

Patients were monitored for the development of human anti-veltuzumab antibodies and human anti-milatuzumab antibodies (HAHA) at the following timepoints: pre-treatment on day 1 of week 1, pre-treatment on day 1 of week 4, pre-treatment on day 1 of week 12, and pre-treatment on day 1 of week 36. HAHA levels were by the study sponsor using an ELISA with a lower level of quantification (LLQ) of 50 ng/ml.

Statistical Methods—

The primary objective of the phase I dose escalation study was to determine the DLT, MTD, and toxicity profile of the combination of veltuzumab and milatuzumab in relapsed and refractory NHL. The phase I study was followed by a phase II study at the recommended phase II dose. The primary endpoint for the phase II study was overall objective response rate, defined as the proportion of patients demonstrating a complete or partial response to treatment per International response criteria (Cheson et al., 2007, J Clin Oncol 25:579-86; Kimby et al., 2006, Clin Lymphoma Myeloma 6:380-83). A Minimax two-stage design (Simon, 1989, Control Clin Trials 10:1-10) was utilized and considered the regimen to be ineffective or uninteresting if the true overall response (OR) rate was less than 10% (p₀). The regimen was deemed worthy of further study if the true OR probability or target OR rate was 35% or greater (p₁). These figures resulted in a Simon's two-stage Minimax design of 8 and 18 patients, with an alpha of 0.10 and beta of 0.10. At this interim analysis after eight patients were enrolled, the endpoint was met and enrollment resumed. After a total 17 patients were accrued to the phase II study, further enrollment was held and the results were analyzed. Summary statistics (i.e. median and range for continuous variables, and frequency for discrete data) were calculated for patient demographics. The maximum grade for each type of toxicity was recorded for each patient, and frequency tables were provided. Progression free survival (PFS) was calculated from the date of start of therapy to disease progression or death, whichever occurred first. PFS was censored at the time of stem cell transplant. Overall survival (OS) was determined from the start of treatment to death from any cause. Patients who were still alive were censored at the date of last follow-up. Survival curves were estimated using the method of Kaplan-Meier. Median PFS and OS were assessed with 95% confidence interval.

Results

Patient Characteristics—

A total of 35 patients were enrolled, including 18 patients in the phase I study and 17 patients in the phase II study. Baseline patient characteristics are summarized in Table 7. Histologies included follicular lymphoma (n=16, 46%), diffuse large B-cell lymphoma (n=7, 20%), mantle cell lymphoma (n=6, 17%), transformed follicular lymphoma (n=1, 3%), marginal zone lymphoma (n=2, 6%), Waldenstrom's macroglobulinemia (n=2, 6%), and lymphoplasmacytic lymphoma (n=1, 3%). Median age was 63 years (range 39-83 years) and median number of prior therapies was 3 (range 1-10). All patients had received prior rituximab and 63% were rituximab refractory. Four patients had relapsed following prior autologous stem cell transplant.

TABLE 7
Baseline patient characteristics

| | Phase I | | Phase II | | All Patients | |
|---|---|---|---|---|---|---|
| Characteristics | (n = 18) | | (n = 17) | | (n = 35) | |
| Median age, years | 65 | (44-81) | 63 | (39-82) | 63 | (39-82) |
| (range) | | | | | | |

| | | | |
|---|---|---|---|
| Gender (M/F) | 13/5 | 10/7 | 23/12 |

Histology

| Follicular lymphoma | 5 | (28%) | 6 | (35%) | 11 | (31%) |
|---|---|---|---|---|---|---|
| Grade 1-2 | | | | | | |
| Follicular lymphoma | 5 | (28%) | 0 | | 5 | (14%) |
| Grade 3 | | | | | | |
| Transformed follicular | 1 | (6%) | 0 | | 1 | (3%) |
| Diffuse large B-cell | 4 | (22%) | 3 | (18%) | 7 | (20%) |
| lymphoma | | | | | | |
| Mantle cell lymphoma | 1 | (6%) | 5 | (29%) | 6 | (17%) |
| Lymphoplasmacytic | 1 | (6%) | 2 | (12%) | 3 | (9%) |
| lymphoma | | | | | | |
| Marginal zone lymphoma | 1 | (6%) | 1 | (6%) | 2 | (6%) |
| Rituximab refractory | 10 | (56%) | 12 | (71%) | 22 | (63%) |
| Bone marrow | 4 | (22%) | 5 | (29%) | 9 | (26%) |
| involvement | | | | | | |
| Prior transplant | 3 | (17%) | 1 | (6%) | 4 | (11%) |

| | | | |
|---|---|---|---|
| LDH (average), normal | 223 | 203 | 213 |
| 190 | | | |

ECOG Performance Status

| 0 | 8 | | 6 | | 14 | |
|---|---|---|---|---|---|---|
| 1 | 9 | | 7 | | 16 | |
| 2 | 1 | | 4 | | 5 | |
| Median number of prior | 3 | (1-9) | 3 | (1-10) | 3 | (1-10) |
| treatments (range) | | | | | | |

Dose Escalation and Safety Evaluation—

No dose limiting toxicities (DLT) were observed, although 3 of the first 6 patients had grade 3 infusion reactions with milatuzumab, consisting of fevers, rigors, nausea, vomiting, diarrhea, and rash requiring hospitalization despite pre-medication and treatment. As a result of the infusional toxicity, the study was amended to remove milatuzumab on day 1 of weeks 2-4, 12, 20, 28, and 36 and add 20 mg dexamethasone immediately prior to and 10 mg dexamethasone post each milatuzumab dose. DLT was amended to include grade 3 infusion reactions requiring hospitalization and grade 4 infusion reactions. Enrollment resumed with three additional patients treated at dose levels 1 and 2 and six patients treated at dose level 3, and no DLTs were observed in the phase I study. A summary of the related toxicities is detailed in Table 8.

TABLE 8
Common Treatment-Related Toxicities in 35
patients on the phase I and II studies

| | | | | |
|---|---|---|---|---|
| Toxicity n = 35 (%) | Grade 2 | Grade 3 | Grade 4 | All |

Hematologic Toxicity

| | | | | |
|---|---|---|---|---|
| Leukopenia | 5 (14) | 3 (9) | 0 | 8 (23) |
| Neutropenia | 1 (3) | 4 (11) | 0 | 5 (14) |
| Lymphopenia | 8 (23) | 5 (14) | 6 (17) | 19 (54) |
| Anemia | 4 (11) | 1 (3) | 0 | 5 (14) |
| Thrombocytopenia | 7 (20) | 0 | 0 | 7 (20) |

Non-hematologic Toxicity

| | | | | |
|---|---|---|---|---|
| Infection | 5 (14) | 0 | 0 | 5 (14) |
| Infusion reactions | 16 (46) | 5 (14) | 0 | 21 (60) |
| Nausea | 2 (6) | 0 | 0 | 2 (6) |
| Vomiting | 1 (3) | 0 | 0 | 1 (3) |
| Diarrhea | 2 (6) | 0 | 0 | 2 (6) |
| Fatigue | 2 (6) | 1 (3) | 0 | 3 (9) |
| Atrial Tachycardia | 0 | 1 (3) | 0 | 1 (3) |
| Hyperglycemia | 2 (6) | 2 (6) | 0 | 4 (11) |
| Abdominal pain | 1 (3) | 0 | 0 | 1 (3) |
| Chills | 2 (6) | 0 | 0 | 2 (6) |
| Dyspnea | 1 (3) | 0 | 0 | 1 (3) |
| Hypophosphatemia | 2 (6) | 0 | 0 | 2 (6) |
| Muscle weakness | 1 (3) | 0 | 0 | 1 (3) |
| Mucositis | 1 (3) | 0 | 0 | 1 (3) |
| Respiratory congestion | 1 (3) | 0 | 0 | 1 (3) |
| Urinary retention | 1 (3) | 0 | 0 | 1 (3) |

Infusion reactions were common, but were generally grade 1-2. Only one patient experienced a grade 3 infusion reaction to milatuzumab after the amendment which occurred on week 28 of extended induction. The patient did not require hospitalization and responded to interventions, but was taken off of study therapy. Hematologic toxicity consisted of grade 3-4 lymphopenia (31%), grade 3 leukopenia (9%), grade 3 neutropenia (11%), and grade 3 anemia (3%). One patient discontinued study therapy for grade 3 neutropenia which occurred on week 20 and was considered likely related to study therapy. A bone marrow biopsy was performed and demonstrated no evidence of NHL or myelodysplasia. The neutropenia resolved by week 23. Infectious complications were all grade 1-2 and included an upper respiratory infection, shingles, sinusitis, pneumonia, a urinary tract infection, and thrush. Common grade non-hematologic toxicities in both the phase I and II studies included nausea, diarrhea, and fatigue.

Delivery of Therapy and Response—

All 35 patients received at least one dose of both veltuzumab and milatuzumab and thirty-four patients were evaluable for response. One patient who experienced a supraventricular tachycardia during week 3 was removed from study therapy and was not assessed for response. Three patients, two with diffuse large B-cell lymphoma and one with mantle cell lymphoma, did not complete induction due to progressive disease. The remaining patients (n=30, 86%) completed at least all 4 weeks of induction therapy. A total of 10 of 35 patients (29%) completed all 36 weeks of therapy including induction and extended induction. The median number of weeks of therapy was 12. Reasons for discontinuation of therapy during extended induction included progression (n=14), toxicity (n=4), and drug supply (n=2). In terms of the toxicities which lead to discontinuation, two patients elected to stop therapy due to recurrent grade 3 infusion reactions despite premedication, at weeks 12 and 28, respectively. One patient, previously mentioned, developed grade 3 neutropenia during week 20 that resolved by week 23, but elected to discontinue therapy. The fourth patient who discontinued treatment due to toxicity had a pulmonary embolism which was unrelated to study therapy and was later diagnosed with myelodysplasia, after 4 prior therapies. One patient remained on follow up for response at the time the data was censored.

Eight of 35 patients achieved a partial or complete response, resting in an overall response rate for the entire evaluable cohort including both the phase I and phase II study participants of 24%. Responses were durable with a median duration of response of 12 months (range 3-23 months). Complete responses were observed in two patients; one patient with grade 1-2 follicular NHL who was rituximab-refractory after three prior therapies who ultimately underwent allogeneic stem cell transplant and one patient with marginal zone lymphoma with one prior therapy. Partial responses were observed in 6 patients with indolent NHL and MCL. Stable disease (SD) was observed in 17 patients with a median duration of 6 months (range 2-22 months). Notably, one patient with follicular lymphoma was removed from study therapy after week 20 of extended induction for progression but subsequent imaging showed regression of the lymph nodes, and the patient has no evidence of disease or additional therapy at a follow up of 44 months.

Of the eight patients with aggressive NHL including seven with diffuse large B-cell lymphoma and one with transformed follicular lymphoma, one patient achieved stable disease at week 5, but progressed prior to extended induction and the remaining patients had progressive disease during induction. Of the patients with indolent NHL and mantle cell lymphoma, the ORR for evaluable patients was 31% with an additional 47% achieving stable disease of 7.5 months median duration. Of the responding patients, 50% were refractory to rituximab. Responses by histology are described in Table 9. Median progression free survival for all patients was 4.7 months (95% CI 2.2, 9.3 months) and overall survival was 27.3 months (95% CI 11.4, 34.0 months).

TABLE 9
Response by histology
Response in Evaluable Patients
N = 34

| Histology | CR | PR | SD | PD | ORR |
|---|---|---|---|---|---|
| Follicular | 1 | 4 | 9 | 1 | 5 (33%) |
| lymphoma | | | | | |
| N = 15 | | | | | |
| Diffuse large | 0 | 0 | 1 | 7 | 0 |
| B-cell lymphoma | | | | | |
| and transformed | | | | | |
| lymphoma | | | | | |
| N = 8 | | | | | |
| Mantle cell | 0 | 1 | 4 | 1 | 1 (17%) |
| lymphoma | | | | | |
| N = 6 | | | | | |
| Lymphoplasmacytic | 0 | 0 | 3 | 0 | 0 |
| lymphoma/ | | | | | |
| Waldenstrom's | | | | | |
| macroglobulinemia | | | | | |
| N = 3 | | | | | |
| Marginal zone | 1 | 1 | 0 | 0 | 2 (100%) |
| lymphoma | | | | | |
| N = 2 | | | | | |
| Total | 2 (6%) | 6 (18%) | 17 (50%) | 9 (26%) | 8 (24%) |
| N = 34 | | | | | |

Pharmacokinetics—

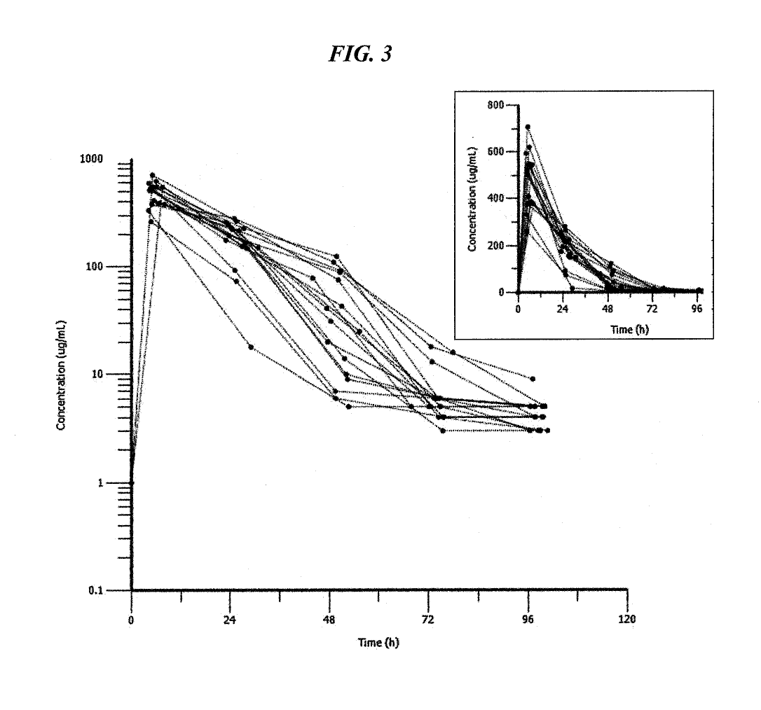

The mean (standard deviation) and median (range) serum veltuzumab levels pre- and post-infusion are summarized in Table 10. Semilog plots of serum veltuzumab concentration versus time at week 4 for patients with evaluable PK profiles are shown in FIG. 2. Table 11 summarizes the overall PK parameters of the 4^{th} veltuzumab infusion. Follicular lymphoma represented the largest cohort in this study (n=16; 46%), and 14 of the 16 patients received the 4^{th} veltuzumab infusion and therefore serum PK samples were collected. Of the 14 patients, NCA and one-compartment PK analysis were performed on 12 evaluable PK profiles. Subgroup veltuzumab PK analysis for follicular lymphoma patients showed that the half-lives of veltuzumab were longer in responders than nonresponders (P=0.002 for terminal phase half-life using NCA; P=0.004 for one-compartment model). No statistical significant differences were observed for AUC, Cₘₐₓ, V and CL, albeit there was a trend towards significance for AUC (P ≦0.1) (Data not shown). Semilog plots of serum milatuzumab concentration versus time at 1^{st} infusion for patients dosed at 20 mg/kg with evaluable PK profiles are shown in FIG. 3. First dose milatuzumab PK parameters using NCA for 17 patients dosed at 20 mg/kg (n=17) are summarized in Table 12. Consistent with previous findings that milatuzumab has relatively short half-life as a monoclonal antibody, likely the result of rapid internalization by cells. The estimated mean half-life of milatuzumab is approximately 10 hours.

TABLE 10
Veltuzumab serum levels at 200
mg/m² dose: pre-dose and post-dose levels

| | |
|---|---|
| Mean (SD) | Median (range) |
| μg/mL | μg/mL |

1^{st} Infusion

| | | |
|---|---|---|
| Pre (n = 32) | 11.2 (24.1) | 1.0 (<0.5-92) |
| Post (n = 32) | 119.5 (53.9) | 111.0 (30-276) |

2^{nd} infusion

| | | |
|---|---|---|
| Pre (n = 30) | 36.1 (27.1) | 35.5 (<0.5-102) |
| Post (n = 29) | 152.4 (57.2) | 144 (59-282) |

4^{th} infusion

| | | |
|---|---|---|
| Pre (n = 28) | 81.8 (59.7) | 74.5 (2-242) |
| Post (n = 27) | 205.7 (88.6) | 201 (40-374) |

Week-12

| | | |
|---|---|---|
| Pre (n = 19) | 20.4 (17.1) | 17.0 (1-62) |
| Post (n = 19) | 122.3 (30.5) | 114 (55-180) |

Week-36

| | | |
|---|---|---|
| Pre (n = 8) | 7.8 (5.7) | 9.5 (<0.5-16) |
| Post (n = 7) | 122.6 (22.4) | 126 (79-150) |

Note:
Most of the pre-dose levels were collected within 2 hours prior to infusion, a few were collected up to 5 hours prior to infusion. Most of the post-dose levels were collected within 30 min of infusion completion, all were collected within 1 hour. SD, standard deviation

Note: Most of the pre-dose levels were collected within 2 hours prior to infusion, a few were collected up to 5 hours prior to infusion. Most of the post-dose levels were collected within 30 min of infusion completion, all were collected within 1 hour. SD, standard deviation

TABLE 11
Veltuzumab pharmacokinetics after fourth infusion (n = 19)

| | | |
|---|---|---|
| Parameter | Mean (SD) | Median (range) |

Noncompartmental analysis

| | | |
|---|---|---|
| AUC_{0-∞} (d*μg/mL) | 4267 (2360) | 3747 (422-8845) |
| Cₘₐₓ (μg/mL) | 228 (76) | 206 (138-373) |
| t_{1/2} (d)* | 16.9 (7.3) | 16.9 (4.3-28.6) |
| Vz (mL) | 2739 (1363) | 2661 (1035-6664) |
| CL (mL/d/m²) | 80.9 (101.7) | 53.4 (22.6-473.9) |

One compartment model

| | | |
|---|---|---|
| AUC_{0-∞} (d*μg/mL) | 3911 (2060) | 3586 (384-7420) |
| Cₘₐₓ (μg/mL) | 206 (70) | 197 (112-371) |
| t_{1/2} (d) | 13.0 (5.4) | 13.3 (1.9-20.4) |
| Vc (mL) | 2139 (805) | 2132 (1015-3457) |
| CL (mL/d/m²) | 86.6 (111.1) | 55.8 (27.0-521.4) |

*terminal phase half life; SD, standard deviation; AUC_{0-∞}, area under the concentration time curve from dosing time extrapolated to infinity; CL, total body clearance; Cₘₐₓ, maximum observed concentration; t_{1/2}, half life; Vz, volume of distribution for terminal phase; Vc, volume of distribution of the central compartment.

terminal phase half life; SD, standard deviation; AUC_{0-∞}, area under the concentration time curve from dosing time extrapolated to infinity; CL, total body clearance; Cₘₐₓ, maximum observed concentration; t_{1/2}, half life; Vz, volume of distribution for terminal phase; Vc, volume of distribution of the central compartment.

Immunogenicity (HAHA)—

Of the 33 patients whose HAHA titers were assayed, only one patient developed human anti-veltuzumab antibodies (HAHA). That patient displayed an atypical veltuzumab PK profile, as denoted in FIG. 1. There were no clinical consequences. None of these patients developed detectable human anti-milatuzumab antibody.

TABLE 12
First dose milatuzumab pharmacokinetics
for patients dosed at 20 mg/kg (n = 17)

| | | |
|---|---|---|
| Parameter | Mean (SD) | Median (range) |

Noncompartmental analysis

| | | |
|---|---|---|
| AUC_{0-∞} (d*μg/mL) | 422 (121) | 417 (162-627) |
| Cₘₐₓ (μg/mL) | 480 (116) | 511 (262-708) |
| t_{1/2} (h)* | 10.3 (2.3) | 10.0 (7.0-15.3) |
| Vz (mL) | 2432 (1100) | 2107 (1497-6051) |
| CL (mL/hr) | 175 (105) | 151 (84-525) |

*terminal phase half life; SD, standard deviation; AUC_{0-∞}, area under the concentration time curve from dosing time extrapolated to infinity; CL, total body clearance; Cmax, maximum observed concentration; t_{1/2}, half life; Vz, volume of distribution for terminal phase.

Discussion

In the current study, we demonstrated the activity and tolerability of combined milatuzumab and veltuzumab in a group of heavily pretreated patients with NHL. The primary toxicity observed was infusion reactions associated with milatuzumab. The addition of scheduled steroids pre-treatment and post-treatment, as well as separating the dosing of the antibodies to different days, mitigated the reactions. After the study amendment, only one patient in the phase II study developed a grade 3 infusion reaction which was reversible and did not require hospitalization. The majority of reactions were manageable grade 1-2 reactions consisting of rigors, nausea, and diarrhea. The rate of infections was low, and all infections were grade 1-2. Preliminary efficacy was observed in patients with indolent and MCL NHL, with an ORR of 31%, and median duration of response of 12 months in these patients. Responses were observed in 4/8 (50%) of rituximab-refractory patients. In the patients with diffuse large B-cell lymphoma and transformed lymphoma, there were no objective responses and several of the patients progressed rapidly during induction. Based on these results, combined milatuzumab and veltuzumab has demonstrated preliminary efficacy in patients with indolent and rituximab-refractory NHL.

Due to the safety and tolerability of combined antibody therapy, the results of this trial and other studies (Leonard et al., 2005, J Clin Oncol 23:5044-51; Leonard et al., 2007, Ann Oncol 18:1216-23; Strauss et al., 2006, J Clin Oncol 24:3880-86; Leonard et al., 2008, Cancer 113:2714-23) support the use of combined monoclonal antibody therapy for indolent NHL subtypes. In the future, combined monoclonal antibody therapy may supplant the use of traditional cytotoxic chemotherapy combinations as front-line and relapsed therapy for indolent NHL due to its limited toxicity and the ability for prolonged maintenance administration.

Further combinations of veltuzumab or milatuzumab with other novel agents that target differing apoptotic pathways designed to minimize toxicity, maximize patient convenience, improve response rate are also under investigation. Specifically, preclinical studies (Alinari et al., 2011, Blood 118:6893-903) demonstrated that FYT720 (fingolimod), an immunosuppressive drug currently FDA approved for the treatment of multiple sclerosis, increases CD74 expression in MCL cell lines by blocking autophagy and lysosomal membrane permeabilization resulting in decreased degradation of CD74. An in vivo preclinical model of MCL in SCID (CB17 scid/scid) mice also demonstrated a statistically significant increase in median survival in the group treated with the combination of FTY720 and milatuzumab compared to control, FTY720 alone, and milatuzumab alone (Alinari et al., 2011, Blood 118:6893-903). Therefore, combination treatment with agents that result in increased expression of CD74 may enhance responses and overcome resistance. Furthermore, preclinical evaluation of two novel bispecific hexavalent antibodies constructed from veltuzumab and milatuzumab demonstrated promising in vitro activity in NHL cell lines and primary patient samples (Gupta et al., 2012, Blood 119:3767-78). As milatuzumab is internalized, exploration of radioimmunotherapy and toxin-antibody conjugates is also under active investigation (Stein et al., 2007, Clin Cancer Res 13:5556s-63s). Milatuzumab is currently being evaluated in a phase I study in combination with standard graft-versus-host disease prophylaxis for patients undergoing allogeneic stem cell transplant for B-cell malignancies, due to its ability to selectively reduce CD74 expressing myeloid dendritic cells (NCT01663766) (Chen et al., 2013, Biol Blood Marrow Transplant 19:28-39).

In conclusion, the efficacy and limited toxicity of veltuzumab and milatuzumab in single agent trials and from our phase I/II trial, as well as the promising in vitro and in vivo synergy when these agents are combined, demonstrate the unexpected superior efficacy of therapy with anti-CD20 (veltuzumab) and anti-CD74 (milatuzumab) antibodies in indolent NHL subtypes, compared to standard therapies such as treatment with rituximab.

### Example 2

### Combination DNL Constructs Comprising Anti-CD74 Antibodies Show Potent Toxicity Against B Cell Lymphoma

### Juxtaposing CD20 and CD74 by Bispecific Antibodies Evokes Potent Cytotoxicity in Mantle Cell Lymphoma

Abstract

We describe the potent growth-inhibitory and apoptotic activities of two bispecific hexavalent antibodies (HexAbs) constructed by the DOCK-AND-LOCK® (DNL®) technique from veltuzumab (anti-CD20) and milatuzumab (anti-CD74) in mantle cell lymphoma (MCL) and other lymphoma/leukemia lines, as well as primary patient tumor samples. In vitro, the bispecific HexAbs had different properties and were more potent than their parental antibodies. The juxtaposition of CD20 and CD74 on MCL cells by the HexAbs resulted in homotypic adhesion and triggered intracellular changes that included loss of mitochondrial membrane potential, production of reactive oxygen species, increased phosphorylation of ERKs and JNK, downregulation of pAkt and Bcl-xL, and enlargement of lysosomes, culminating in cell death. The two HexAbs also displayed different potencies in depleting lymphoma cells from whole blood ex vivo, and significantly extended the survival of nude mice bearing MCL xenografts.

Introduction

Mantle cell lymphoma (MCL) is an aggressive subtype of B-cell non-Hodgkin lymphoma (NHL) generally having a poor prognosis. Currently, there is no established standard of care and relapsed MCL remains a major clinical challenge. Thus, there is a need to develop new targeted therapeutics to treat this disease (Diefenbach & O'Connor, 2010, Curr Opin Oncol. 22:419-423).

Monoclonal antibodies (MAbs), exemplified by rituximab, are among the treatment options for MCL (Weigert et al., 2009, Leuk Lymphoma 50:1937-1950; Zhou et al., 2007, Am J Hematol 83:144-149) and have shown encouraging results in MCL (Lenz et al., 2005, J Clin Oncol 23:1984-1992; Sachanas et al., 2011, Leuk Lymphoma 52:387-93). However, resistance to rituximab therapy remains a problem (Lim et al., 2011, Blood July 18, Epub ahead of print) and more effective methods of treatment for MCL are needed.

The combination of two different targeting MAbs to achieve improved efficacy without increased toxicity was shown in NHL patients receiving both rituximab and epratuzumab (humanized anti-CD22 IgG₁, also referred to as hLL2) (Leonard et al., 2005, J Clin Oncol 23:5044-5051; Strauss et al., 2006, J Clin Oncol 24:3880-3886; Leonard et al., 2008, Cancer 113:2714-2723). More recently, the potential advantage of targeting both CD20 and CD74 was reported in a preclinical study involving rituximab and milatuzumab (humanized anti-CD74 IgG₁, also referred to as hLL1) which, in the presence of a crosslinking antibody, showed superior anti-tumor activity in MCL lines and primary patient samples than either parental antibody alone (Alinari et al., 2011, Blood 117:4530-41; Alinari et al., 2008, Blood 112:Abstract 886). In principle, combination antibody therapy can be accomplished with a bispecific antibody (bsAb) to avoid the need for administering two different antibodies sequentially, which is time-consuming, expensive, and inconvenient.

The potential of bsAbs as novel therapeutics for cancer and autoimmune disease is being explored with various constructs differing in design, structure, and antigen-binding properties. Depending on the built-in dual specificity, a bsAb may serve to recruit effector cells or effector molecules to target cells, or it may improve the target selectivity by concurrent ligation of two different antigens expressed on the same cell, wherein a multivalent bsAb should also enhance its functional affinity, resulting in increased retention on the bound cells and, likely, a higher potency.

The DOCK-AND-LOCK® (DNL®) method, described above, is a platform technology that combines genetic engineering with site-specific conjugation to enable self-assembly of two modular components only with each other, resulting in a covalent structure of defined composition with retained bioactivity. We have applied DNL® to generate various multivalent, multispecific structures that include mono- and bi-specific HexAbs, each comprising a pair of stabilized dimers of Fab linked to a full IgG, thus conferring six Fab-arms and a common Fc entity. To identify these HexAbs, each is assigned a code of X-(Y)-(Y), where X and Y are specific numbers given to differentiate the antibodies, and a designated number enclosed in a parenthesis representing the antibody as a Fab. For example, 20-(74)-(74) designates the bispecific HexAb comprising a divalent anti-CD20 IgG of veltuzumab (also referred to as hA20) and a pair of stabilized dimers of Fab derived from milatuzumab. The designations of various HexAbs relevant to this study, which include 20-(74)-(74), 74-(20)-(20), 74-(74)-(74), 20-(20)-(20), 20-(22)-(22), and 22-(20)-(20), along with their modular components, are provided in Table 13.

TABLE 13
Hexavalent Antibody Designations

| | | CH₃-AD2-IgG | | CH₁DDD2-Fab | |
|---|---|---|---|---|---|
| Designation | | module | | module | |
| Current | Previous | Ab | Ag | Ab | Ag |
| 20-(74)-(74) | N/A | Veltuzumab | CD20 | Milatuzumab | CD74 |
| 74-(20)-(20) | N/A | Milatuzumab | CD74 | Epratuzumab | CD20 |
| 74-(74)-(74) | N/A | Milatuzumab | CD74 | Milatuzumab | CD74 |
| 20-(20)-(20) | 20-20 | Veltuzumab | CD20 | Veltuzumab | CD20 |
| 20-(22)-(22) | 20-22 | Veltuzumab | CD20 | Epratuzumab | CD22 |
| 22-(20)-(20) | 22-20 | Epratuzumab | CD22 | Veltuzumab | CD20 |

In our initial efforts to develop HexAbs from veltuzumab and epratuzumab, we found both 20-(22)-(22) and 22-(20)-(20) induced growth inhibition and apoptosis in Burkitt lymphoma lines (Daudi, Raji and Ramos) in the absence of a crosslinking antibody, which is often required for the parental antibodies to be effective in vitro (Rossi et al., 2008, Cancer Res 68:8384-8392; Rossi et al., 2009, Blood 113:6161-6171; Gupta et al., 2010, Blood 116:3258-3267). Such direct cytotoxicity, however, was not observed in JeKo-1, a MCL line expressing comparable levels of CD20 and CD22 as Daudi NHL.

In the present Example, we describe the generation and characterization of three novel HexAbs, 20-(74)-(74), 74-(20)-(20), and 74-(74)-(74), from veltuzumab (hA20) and milatuzumab (hLL1). Surprisingly, even though mantle cell lymphoma was resistant to anti-CD20/CD22 HexAbs and to the parental anti-CD74 and anti-CD20 antibodies, the HexAbs based on the combination of anti-CD74 and anti-CD20 antibodies were highly cytotoxic in three blastoid MCL lines, JeKo-1, Granta-519 and Mino, as well as in primary tumor cells from patients with MCL or chronic lymphocytic leukemia (CLL). Selective experiments performed to investigate the intracellular events triggered by juxtaposing CD20 and CD74 revealed the prominent roles of actin reorganization and lysosomal membrane permeabilization (LMP) in the mechanisms of cell death.

Methods

Cell Lines, Antibodies, and Reagents—

All cell lines were purchased from ATCC (Manassas, Va.). Humanized antibodies, including veltuzumab, milatuzumab, epratuzumab, labetuzumab (anti-CEACAM5 IgG₁, also referred to as hMN-14), and hRS7 (anti-human Trop-2 IgG₁), were obtained from Immunomedics, Inc. Tositumomab and rituximab were obtained commercially. Phospho-specific antibodies and other commercially available antibodies were acquired from Cell Signaling (Beverly, Mass.) or Santa Cruz Biotechnology (Santa Cruz, Calif.). Cell culture media, supplements, annexin V ALEXA FLUOR® 488 conjugate, tetramethylrhodamine ethyl ester (TMRE), LYSOTRACKER® Red DND-99, CM-H₂DCF-DA, DAPI, ALEXA FLUOR® phalloidin, and acridine orange were bought from Invitrogen (Carlsbad, Calif.). One Solution Cell Proliferation assay (MTS) was obtained from Promega (Madison, Wis.). PHOSPHOSAFE™ buffer, latrunculin B, cytochalasin D, bafilomycin Al and concanamycin A were procured from EMD chemicals (Gibbstown, N.J.). MAGIC RED′ Cathepsin B assay kit was purchased from ImmunoChemistry Technologies (Bloomington, Minn.). All other chemicals were purchased from Sigma (St. Louis, Mo.).

Cell Culture—

Malignant cell lines were cultured at 37° C. in 5% CO₂ in RPMI 1640 medium supplemented with 10% heat-inactivated fetal bovine serum, 2 mM L-glutamine, 200 U/ml penicillin and 100 μg/ml streptomycin. Cells from CLL and MCL patients were collected from whole blood by Ficoll-Hypaque separation and grown in RPMI media as described for the cell lines.

Cell Proliferation Assay—

Cells were seeded in 48-well plates (5×10⁴ cells per well) and incubated with each test article at a final concentration of 0.006 to 100 nM for 4 days. The number of viable cells was then determined using the MTS assay per the manufacturer's protocol, plotted as percent of the untreated, and analyzed by Prism software.

Effector Function Assays—

ADCC was performed as described previously (Rossi et al., 2008, Cancer Res 68:8384-8392), using JeKo-1 as the target cell and freshly isolated peripheral blood mononuclear cells as the effector cells. To perform CDC, cells were seeded in black 96-well plates at 5×10⁴ cells in 50 μl per well and incubated with serial dilutions (concentration range 3.3×10⁻⁸ to 2.6×10⁻¹⁰M) of test articles in the presence of human complement (1/20 final dilution, Quidel Corp., San Diego, Calif.) for 2 h at 37° C. and 5% CO₂. Viable cells were then quantified using the MTS assay. Controls included cells treated with 0.25% Triton X-100 (100% lysis) and cells treated with complement alone (background).

Annexin V Binding Assay—

Cells in 6-well plates (2×10⁵ cells per well) were treated with each test article at 10 nM for 24 to 48 h, washed, resuspended in 100 μl of annexin-binding buffer (10 mM HEPES, 140 mM NaCl and 2.5 mM CaCl₂ in PBS), stained with 5 μl of Annexin V-ALEXA FLUOR® 488 conjugate for 20 min, then with 1 μg/ml of propidium iodide (PI) in 400 μl of annexin binding buffer, and analyzed by flow cytometry (FACSCALIBUR®, Becton Dickinson, San Jose, Calif.). Cells stained positive with annexin V (including both PI-negative and PI-positive) were counted as apoptotic populations. When required, cells were pretreated with the indicated inhibitors for 2 h before adding the test article.

Immunoblot Analysis—

JeKo-1 cells (6×10⁶ cells in 6 ml) were treated with each test article at 10 nM for a predetermined time. Cells were washed with PBS, centrifuged, and lysed in ice-cold PHOSPHOSAFE™ buffer followed by centrifugation at 13,000×g. Supernatants were collected and protein samples (20 μg) were separated by SDS-PAGE on 4-20% gradient Tris-glycine gels followed by transfer onto nitrocellulose membranes (Bio-Rad, Hercules, Calif.), which were probed with suitable antibodies and developed as described (Rossi et al., 2009, Blood113:6161-6171).

Nuclear Extracts—

JeKo-1 cells (6×10⁶ cells in 6 ml) were treated with each test article at 10 nM for 72 h. Cells were collected and cytosolic and nuclear extracts were obtained as described (Mayo et al., 2001, Methods Enzymol 333:73-87). Equal amounts of nuclear and cytosolic proteins (10 μg) were separated on SDS-PAGE and analyzed for NF-κB protein p65, Brg-1 and β-actin, with the latter two serving as loading controls for nuclear and cytosolic proteins, respectively.

Assessment of Δ_{ψm} and ROS—

Flow cytometry was used to determine Δ_{ψm} and ROS. Briefly, cells in 6-well plates (2×10⁵ cells per well) were treated with each test article at 10 nM for 48 h, washed, stained for 30 min in the dark at 37° C. with TMRE (50 nM) for Δ_{ψm} or CM-H₂DCF-DA (1 μM) for ROS. Samples were then washed with PBS and analyzed.

Assessment of Lysosomal Changes and Cathepsin B Release—

To determine the changes in lysosomal volumes, 2×10⁵ cells per well in 6-well plates were treated with each test article at 10 nM for 48 h. After washing, cells were labeled with LYSOTRACKER® Red DND-99 (75 nM) followed by incubation in the dark at 37° C. for 1 h. Cells were washed with PBS and samples were analyzed by flow cytometry. To evaluate lysosomal membrane permeabilization, JeKo-1 cells were treated with select antibodies at 10 μg/ml for 4 h, labeled with acridine orange, and examined under a fluorescence microscope. To study cathepsin B release, JeKo-1 cells were treated with select antibodies (10 nM) for 48 h, fixed with 4% paraformaldehyde, permeabilized with 0.1% tritonX-100, costained with MAGIC RED™ Cathepsin B and DAPI, and examined under a fluorescence microscope.

Effect on Actin—

Cells were treated with various antibodies or combinations of antibodies as indicated, stained for 30 min with either rhodamine phalloidin and DAPI for actin and nucleus, respectively, or rhodamine phalloidin and FITC-conjugated, Fc fragment-specific, goat anti-human (GAH) antibody (for the location of test antibodies), and visualized under a fluorescence microscope after washing.

Ex Vivo Depletion of JeKo-1 and B Cells from Whole Blood—

JeKo-1 cells (5×10⁴) were mixed with heparinized whole blood (150 μl) from healthy volunteers and incubated with varying concentrations of each test article for 2 d at 37° C. and 5% CO₂. After lysing the red blood cells and washing, the remaining cells were stained with FITC-anti-CD19, PE-anti-CD14 or allophycocyanin (APC)-conjugated mouse IgG₁ isotype control, and analyzed by flow cytometry. JeKo-1 cells and monocytes were identified in the monocyte gate as CD 19⁺ and CD14⁺ populations, respectively. Normal B cells are CD 19⁺ in the lymphocyte gate.

In Vivo Efficacy—

Female 8-week-old SCID mice (Taconic Farms; Germantown, N.Y.) were used. Seven different treatment groups of eight mice each were inoculated i.v. with JeKo-1 (2.5×10⁷ cells). After seven days, one group received 370 μg of 20-(74)-(74) i.p. twice weekly for two weeks. A second group received 74-(20)-(20) with the same dose and schedule. Two lower doses (37 μg and 3.7 μg) also were examined for each HexAb with the same schedule and injection route. The control group received saline. The mice were observed daily for signs of distress or paralysis, weighed weekly, and killed humanely when they developed hind-limb paralysis, became moribund, or lost more than 20% of initial body weight.

Statistical Analyses—

For in vitro studies, the statistical difference between two populations was determined by Student's t-test. For in vivo studies, statistical differences in survival between treatment groups were analyzed using Kaplan-Meier plots provided by Prism software. P<0.05 was considered statistically significant.

Results

Generation of HexAbs and Demonstration of Direct Cytotoxicity In Vitro—

The generation of monospecific and bispecific HexAbs by the DNL® method from the cognate C_{H}3-AD2-IgG-X and C_{H}1-Fab-DDD2-Y, where X and Y can be either hLL1 (milatuzumab) or hA20 (veltuzumab), was performed as previously described (see, e.g., U.S. Pat. No. 7,527,787). The HexAbs were purified to near homogeneity, as indicated by SDS-PAGE and SE-HPLC analyses (not shown) for 20-(74)-(74) and 74-(20)-(20). Both also showed stronger binding to three MCL cell lines (JeKo-1, Granta-519 and Mino) than their parental antibodies (data not shown).

In the cell proliferation assays (FIG. 4A), the two bispecific anti-CD20/CD74 HexAbs demonstrated potent cytotoxicity against JeKo-1, Granta-519, Mino, and Raji, with the half maximal effector concentration (EC₅₀) in the low nanomolar range. In comparison, we observed <20% (Granta-519 and Raji) and <50% (Jeko-1 and Mino) growth inhibition when both parental antibodies were combined at the highest concentration tested (100 nM) (FIG. 4A). Additional results shown in FIG. 4B revealed that the anti-proliferative activity of the monospecific 74-(74)-(74) and 20-(20)-(20) HexAbs, as well as the bispecific anti-CD20/CD22 HexAb, 20-(22)-(22), paralleled that of combined hLL1 and hA20 in JeKo-1, and thus was considerably lower in activity than the bispecific anti-CD20/CD74 HexAbs.

On the other hand, notable cytotoxicity of 20-(20)-(20) was found in Mino and Granta-519, and an initial survey showed cell lines derived from CLL (WAC and MEC-1), acute lymphocytic leukemia (REH-1 and MN60), and multiple myeloma (CAG, RPMI8266, KMS11, KMS12-BM, and KMS12-PE) were relatively resistant to the HexAbs. For both 20-(74)-(74) and 74-(20)-(20), the addition of either parental antibody at a moderate concentration of 10 μg/mL partially reduced their anti-proliferative effects in JeKo-1 (FIG. 4C). A list of the EC₅₀ values (nM), as determined by the MTS assay, is provided in Table 14. Surprisingly, DNL® constructs comprising anti-CD74 and anti-CD20 antibodies or fragments thereof were substantially more effective at treating mantle cell lymphoma than anti-CD20/anti-CD22 DNL constructs or either parental antibody administered alone or together.

TABLE 14
EC₅₀ values (nM) for HexAbs comprising hA20 and hLL1 in three MCL (JeKo-1, Granta-519, Mino) and
2 Burkitt lymphoma lines (Daudi, Raji). The EC₅₀ values as determined by the MTS assay for 20-
(74)-(74) and 74-(20)-(20) in two CLL lines (WAC, MEC-1), 2 ALL lines (REH-1, MN60), and 5 MM
lines ((CAG, RPMI8226, KMS11, KMS12-BM, KMS12-PE), were all greater than 100 nM. In MM lines,
hLL1, hA20, hLL1 + GAH, hA20 + GAH, and hLL1 + hA20 + GAH show no anti-proliferative effect.

| | EC₅₀ (nM) | | | | | Percent inhibition (%) at 100 nM | | | |
|---|---|---|---|---|---|---|---|---|---|
| | JeKo-1 | Granta-519 | Mino | Daudi | Raji | JeKo-1 | Granta-519 | Mino | Raji |
| hLL2 | | | | | | | | | |
| hLL1 | | | | | | 0 | 0 | | |
| hA20 | | | | | | 0 | 8 | | |
| Rituximab | | | | | | 0 | 11 | | |
| hLL2 + GAH | | | | | | | | | |
| hLL1 + GAH | | | | | | 36 | 38 | | |
| hA20 + GAH | | | | | | 27 | 37 | | |
| Rituximab + GAH | | | | | | 20 | 45 | | |
| hA20 + hLL1 | >100 | >100 | >100 | | >100 | 0 | 34 | | |
| Rituximab + hLL1 | | | | | | 0 | 40 | | |
| hA20 + hLL1 + GAH | | | | | | 68 | 61 | | |
| Rituximab + hLL1 + GAH | | | | | | 67 | 60 | | |
| 20-(74)-(74) | 3 | 2 | 13.7 | 5.3 | 0.3 | 70 | 55 | 80 | 50 |
| 74-(20)-(20) | 2 | 0.6 | 1.2 | 1.2 | 0.3 | 90 | 65 | 100 | 50 |
| 74-(74)-(74) | >100 | | | | | | | | |
| 20-(20)-(20) | >100 | 0.2 | 3.4 | 5.8 | 8 | | | | |
| 22-(20)-(20) | | | | | | | | | |
| 20-(22)-(22) | >100 | | | | | | | | |

Antibody-Dependent Cellular Cytotoxicity (ADCC)—

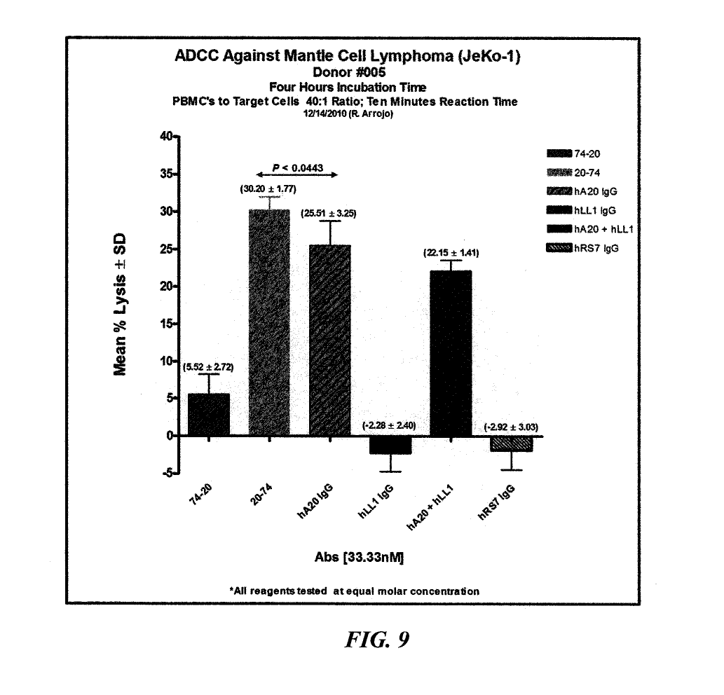

HexAbs based on anti-CD20 IgG, i.e., the 20-(X)-(X) series, were expected to display ADCC with a similar potency to hA20 IgG (Rossi et al., 2008, Cancer Res 68:8384-8392; Rossi et al., 2009, Blood 113:6161-6171). As shown in FIG. 9, the mean cell lysis obtained in JeKo-1 with 20-(74)-(74) dosed at 33 nM, with a 40 to 1 effector to target cell ratio, was 30.2%, slightly higher but statistically significantly (P<0.0443) more than hA20 IgG (25.5%). Under the same conditions, a much weaker ADCC (5.5% lysis) was observed for 74-(20)-(20), whereas hLL1 IgG or a non-binding control (hRS7 IgG) had no ADCC.

Complement-Dependent Cytotoxicity (CDC)—

The results of CDC were determined for JeKo-1, Granta-519, and Daudi cells. In all three cell lines, 74-(20)-(20) displayed negligible CDC (not shown), whereas 20-(74)-(74) was moderately active in Jeko-1 and Granta-519, but as potent as hA20 IgG in Daudi (not shown).

Apoptosis—

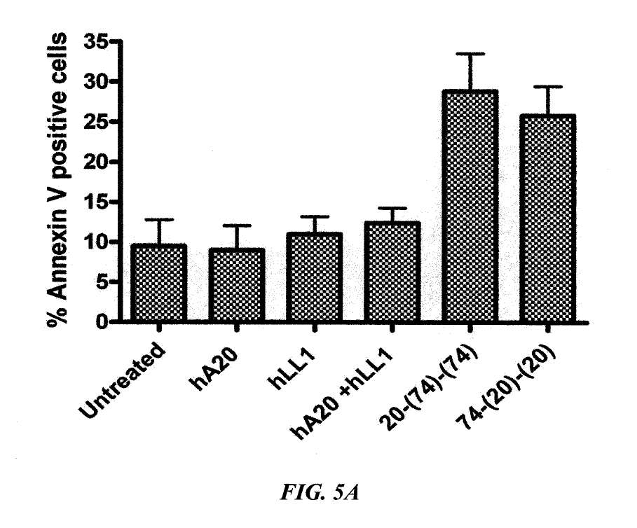

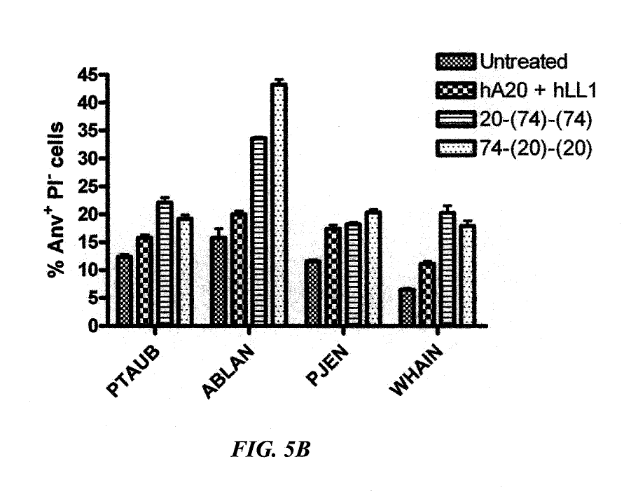

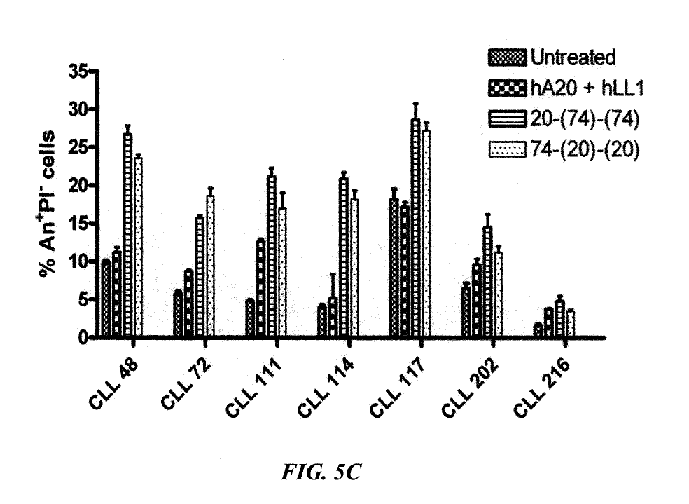

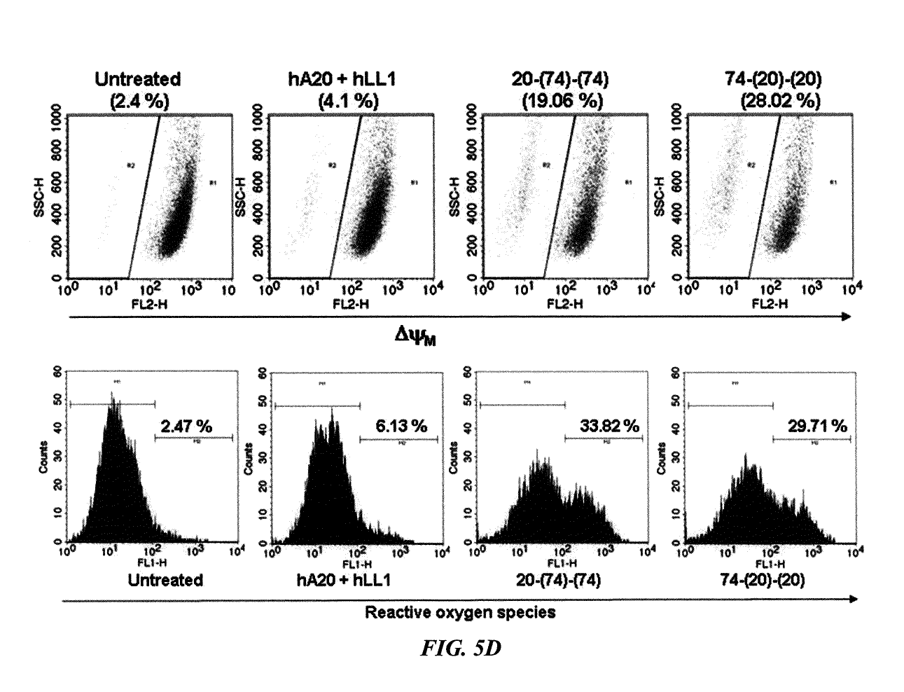

The ability of 20-(74)-(74) and 74-(20)-(20) to induce apoptosis was evaluated by flow cytometry using the Annexin V binding assay. In JeKo-1, both bispecific anti-CD20/CD74 HexAbs at 10 nM showed a statistically significant (P<0.033) increase of 10-15% in Annexin V-positive cells over the various controls (FIG. 5A), which included untreated cells, cells treated with either parental antibody, and cells treated with both parental antibodies combined. Statistically significant increases of 10 to 25% in annexin V-positive cells were also observed for all four MCL patient samples (P<0.008; FIG. 5B) and six of seven CLL patient samples (P<0.03, FIG. 5C). Immunoblot results indicated that the treatment of JeKo-1 with the bispecific anti-CD20/CD74 HexAbs had no apparent effect on the expression levels of Bcl-2, Mcl-1, and Bax, but substantially reduced the amount of Bcl-xL (not shown). The lack of appreciable change in the expression level and cleavage of caspase 3, caspase 8, and caspase 9 (not shown) suggest that the bispecific anti-CD20/CD74 mediate a caspase-independent apoptosis, which was associated with 20 to 30% changes in mitochondrial membrane potential (Δ_{ψm}) and about 30% increase in ROS in Granta-519 (FIG. 5D), and somewhat less in JeKo-1 (not shown).

Homotypic Adhesion and Actin Reorganization—

The 20-(74)-(74) and 74-(20)-(20) hexavalent DNL constructs, but not the parental antibodies, evoked in JeKo-1 a strong homotypic adhesion (not shown), which was prevented by latrunculin B, an inhibitor of actin polymerization. Similar results were observed for Granta-519, Mino and Raji (data not shown). Significant homotypic adhesion (>40%) also could be induced in Jeko-1, but not in KMS-11 (a CD20-negative multiple myeloma line expressing a high level of CD74), by tositumomab (murine anti-human CD20 IgG_{2b}) alone, or by the parental antibodies in the presence of a crosslinking antibody (Table 15). JeKo-1 and KMS-11 cells (2×10⁶/mL) were incubated with the indicated treatments for 2 and 24 h at 37° C. The values (%) shown were the mean from three different fields. KMS-11 is a multiple myeloma cell line with low CD20 expression and high CD74. nd, not determined.

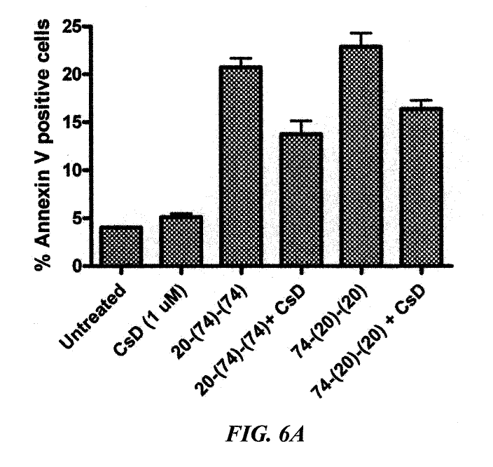

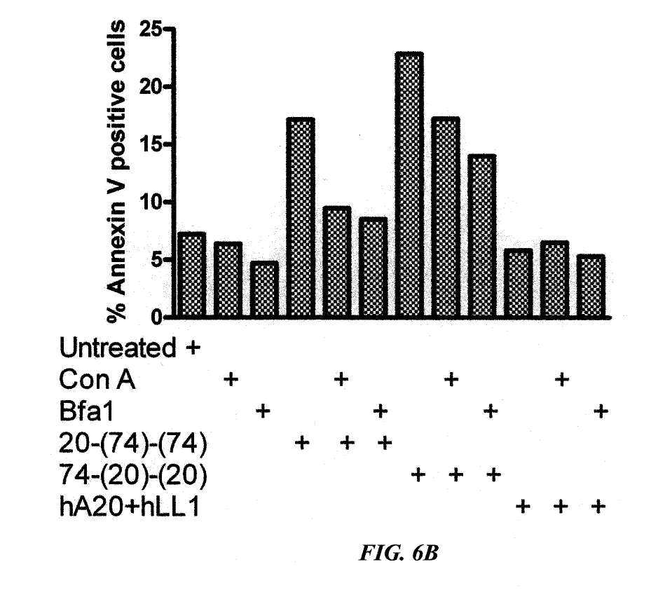

Pretreatment of JeKo-1 with cytochalasin D, which is less toxic than latrunculin B and allows a longer period of incubation, decreased the extent of annexin V-positive cells (FIG. 6A) from 21±1% to 13±2% (P<0.02) by 20-(74)-(74) or from 23±2% to 16±1% (P<0.025) by 74-(20)-(20). These results correlate homotypic adhesion and actin reorganization with apoptosis in cell lines sensitive to treatment with the bispecific anti-CD20/CD74 HexAbs. Additional studies in JeKo-1 revealed that treatment with 20-(74)-(74) or 74-(20)-(20) induced actin to cluster at the cell-cell junction (not shown) and similar results could be produced either with B1 in the absence of a crosslinking antibody, or with rituximab, veltuzumab, or milatuzumab in the presence of a crosslinking antibody (not shown). However, neither 20-(74)-(74) nor 74-(20)-(20) appeared to co-localize with actin at the cell-cell junction when cells were co-stained with FITC-conjugated anti-human-Fc (not shown).

TABLE 15
Homotypic adhesion induced in JeKo-1 by various
antibodies and combinations of antibodies.

| | | JeKo-1 | | KMS-11 | |
|---|---|---|---|---|---|
| | | 2 h | 24 h | 2 h | 24 h |
| | μg/mL | | | | |
| Untreated | — | 5 ± 2 | <8 | <2 | <4 |
| GAH | 20 | 6 ± 3 | <8 | <3 | <4 |
| hLL2 | 5 | 5 ± 2 | <8 | <2 | <5 |
| hLL1 | 5 | 5 ± 3 | <8 | <3 | <5 |
| hA20 | 5 | 5 ± 3 | <14 | <2 | <4 |
| Rituximab | 5 | 10 ± 4 | <18 | <2 | <5 |
| B1 | 5 | 95 ± 4 | nd | nd | nd |
| hLL2 + GAH | 5 + 20 | 4 ± 2 | <7 | <2 | <8 |
| hLL1 + GAH | 5 + 20 | 8 ± 3 | >95 | <2 | <6 |
| hA20 + GAH | 5 + 20 | 55 ± 10 | >95 | <4 | <4 |
| Rituximab + GAH | 5 + 20 | 42 ± 8 | >95 | <4 | <7 |
| hA20 + hLL1 | 5 + 5 | 10 ± 5 | <18 | <4 | <4 |
| Rituximab + hLL1 | 5 + 5 | 21 ± 5 | <24 | <3 | <8 |
| hA20 + hLL1 + GAH | 5 + 5 + 20 | 60 ± 4 | >95 | <4 | <9 |
| Rituximab + hLL1 + GAH | 5 + 5 + 20 | 56 ± 12 | >95 | <2 | <8 |
| | (nM) | | | | |
| B1 | 10 | 90 ± 5 | >95 | <3 | <9 |
| 20-(74)-(74) | 10 | 90 ± 5 | >95 | <4 | <8 |
| 74-(20)-(20) | 10 | 90 ± 5 | >95 | <2 | <9 |
| 74-(74)-(74) | 10 | nd | nd | nd | nd |
| 20-(20)-(20) | 10 | 16 ± 2 | <21 | <3 | <5 |
| 22-(20)-(20) | 10 | 18 ± 4 | <20 | <3 | <8 |
| 20-(22)-(22) | 10 | 15 ± 4 | <24 | <2 | <6 |

Involvement of Lysosomes—

HexAb-mediated apoptosis could be reduced effectively with concanamycin A or bafilomycin Al, both functioning by blocking lysosomal acidification through selective inhibition of the V-type ATPase (Drose & Altendorf, 1997, J Exp Biol 200:1-8). As shown in FIG. 6B, treatment of JeKo-1 with concanamycin A (10 nM) or bafilomycin Al (50 nM) before the addition of either 20-(74)-(74) or 74-(20)-(20) largely decreased the extent of annexin V-positive cells. Further, a sizable enlargement of the lysosomal compartments by the two bispecific anti-CD20/CD74 HexAbs was demonstrated using flow cytometry with a fluorescent acidotropic probe, LYSOTRACKER® Red DND-99 (not shown). These results implicate a causal role of lysosomes in cell death, and are supported by fluorescence microscopic evidence of lysosomal membrane permeabilization and release of cathepsin B into the cytosol (not shown).

Effect on MAP Kinases, Src, p65/NF-κB, and Akt—

The bispecific anti-CD20/CD74 HexAbs induced rapid and sustained activation of ERK and JNK kinases in Jeko-1. For 74-(20)-(20) and 20-(74)-(74) phosphorylated ERKs and INK, respectively, could be detected within 30 min (not shown), which persisted for the next 6 h and returned to basal levels by 24 h (not shown). In contrast, no phosphorylation of ERKs and INK was observed in those cells incubated with both parental antibodies (not shown). Due to the high expression of the p38 MAP kinase in Jeko-1, we were unable to determine whether its level had changed upon treatment with the bispecific HexAbs.

Select signals upstream and downstream of ERKs and INK were also found to be modulated by treating JeKo-1 with the bispecific anti-CD20/CD74 HexAbs. Pertinent results include the ability of both HexAbs to induce a larger decrease (˜60%) of the upstream phospho-Src than that of the parental hA20 MAb (˜30%) (not shown), and the effective inhibition of the downstream p65/NF-κB from nuclear translocation by both HexAbs (not shown). We also observed that the bispecific HexAbs notably reduced the level of constitutively activated Akt in JeKo-1 (not shown).

Ex Vivo Depletion of JeKo-1 and Normal B Cells from Human Whole Blood—

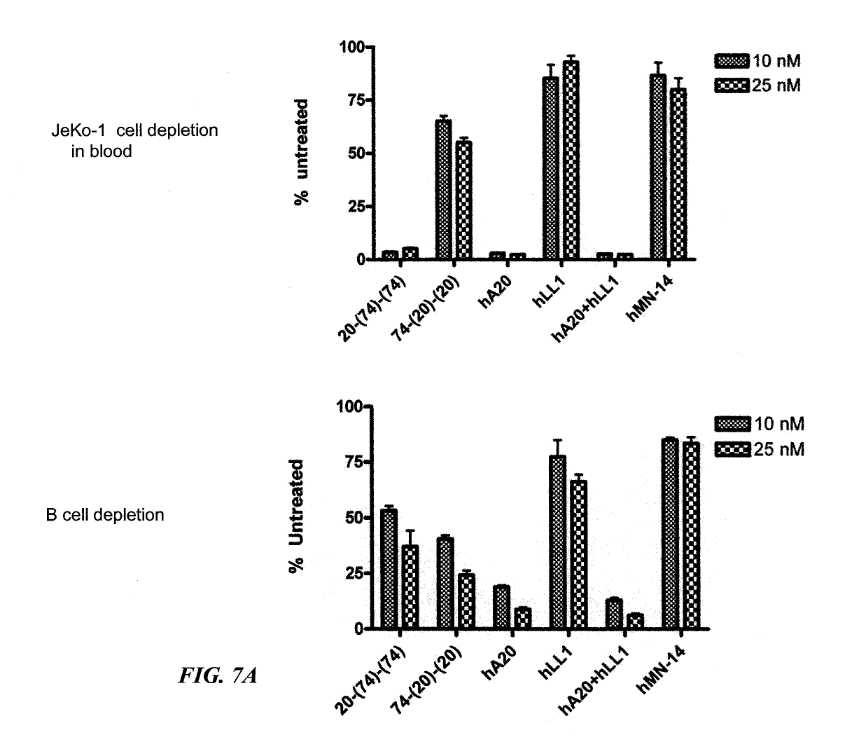

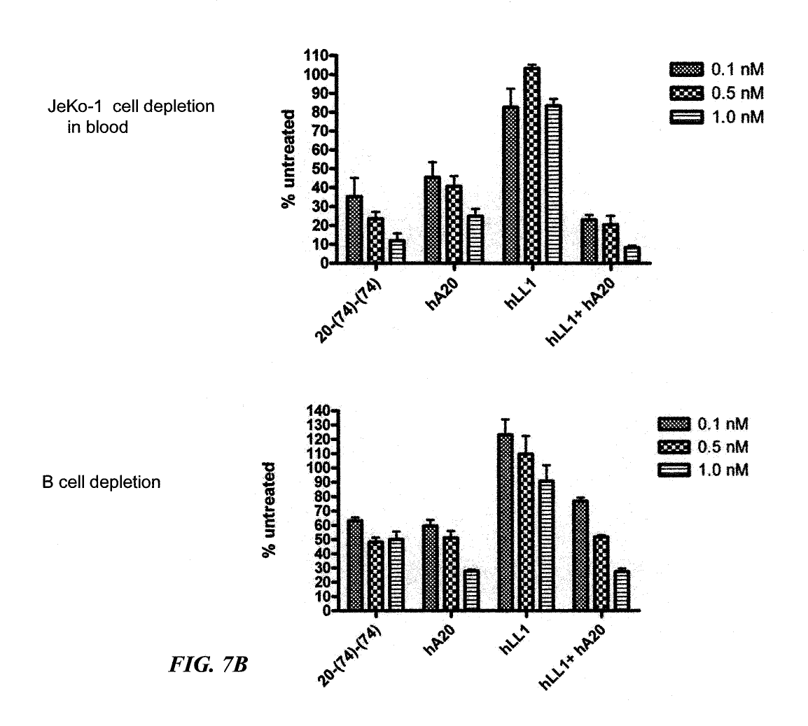

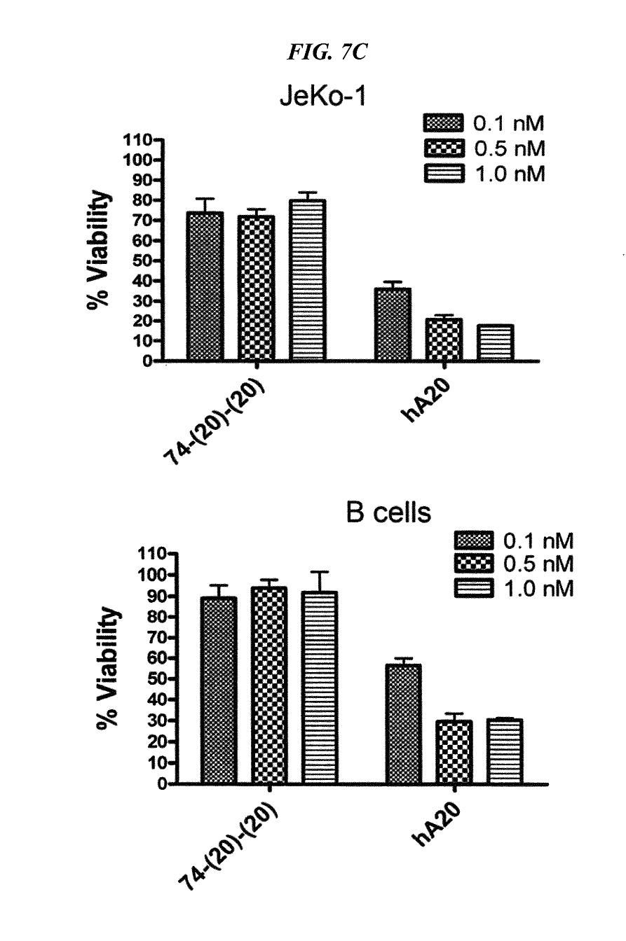

When evaluated at high concentrations (10 and 25 nM) in whole blood spiked with JeKo-1 cells, we observed >90% depletion of JeKo-1 cells for 20-(74)-(74), hA20, and hA20+hLL1, but no more than 50% depletion for 74-(20)-(20), and <20% depletion for hLL1 or the non-targeting hMN-14 (anti-CEACAM5) control antibody (FIG. 7A. upper panel). The depletion of normal B cells, on the other hand, was similar for the two HexAbs, in the range of 50 to 70% for 20-(74)-(74) and 60 to 75% for 74-(20)-(20), with higher depletion (80 to 90%) attained by hA20 or the combination of hA20 and hLL1 (FIG. 7A, lower panel). The apparently higher potency of 20-(74)-(74) as compared to 74-(20)-(20) in depleting JeKo-1 cells from whole blood prompted subsequent studies using three lower concentrations (0.1, 0.5 and 1 nM), and the results shown in FIG. 7B (upper panel) and FIG. 7C (upper panel) confirmed the higher activity of 20-(74)-(74) than 74-(20)-(20) in depleting JeKo-1 cells from whole blood, since 20-(74)-(74) at 0.1 nM was able to deplete 70% of JeKo-1 cells, whereas 74-(20)-(20) at 25 nM could not achieve more than 50% depletion. The high potency of 20-(74)-(74) was also manifested by its ability to deplete 40 to 50% of normal B cells at 0.1 to 1 nM (FIG. 7B, lower panel), a blood concentration that should be easily attainable clinically. Under the same conditions, 74-(20)-(20) at 0.1 to 1 nM depleted 10% or less of normal B cells (FIG. 7C, lower panel). It is noted that the ability of 20-(74)-(74) to deplete either JeKo-1 or normal B cells from whole blood is comparable, not superior, to that of hA20 or the combination of hA20 and hLL1.

In Vivo Studies—

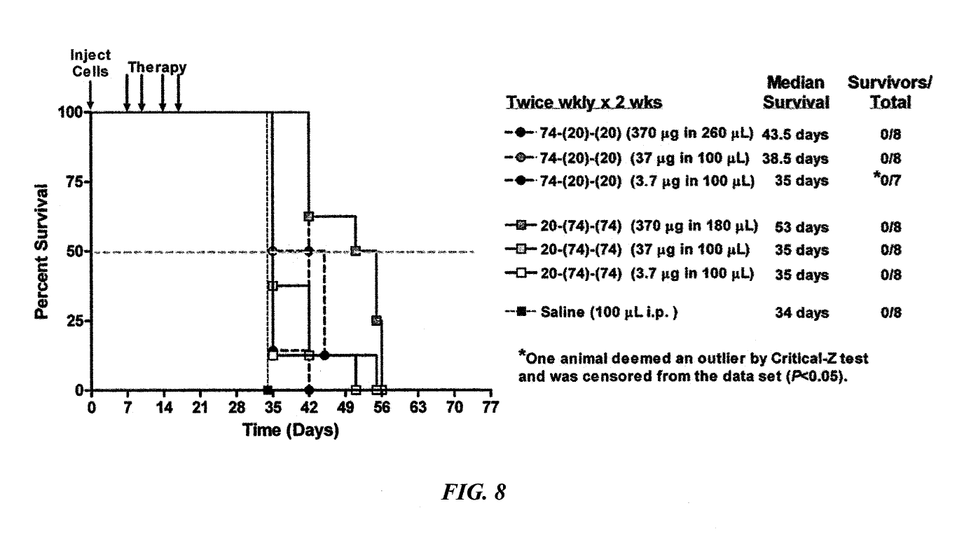

To evaluate the efficacy of HexAbs in vivo, mice bearing disseminated JeKo-1 were treated with increasing doses (3.7, 37, or 370 μg) of either 20-(74)-(74) or 74-(20)-(20) given twice weekly for two weeks. Survival curves for the various treatment groups are shown in FIG. 8. Saline control mice succumbed to disease progression by day 34. Both treatments at all three doses significantly improved survival compared to the control animals (P=0.0001). Mice treated with the highest dose of 74-(20)-(20) had an approximate 30% increase in median survival over saline controls (43.5 days vs. 34 days; P=0.0001). A 60% increase in median survival (MST=53 days) over saline controls was observed in mice treated with 20-(74)-(74) at 370 μg (P=0.0001). Both HexAbs given at 370 μg were more effective than the two lower doses (P<0.0143). However, there were no significant differences between mice treated with 20-(74)-(74) and 74-(20)-(20) at the same dose.

Discussion

CD74 is the cell surface form of the HLA class II-associated invariant chain, which plays a key role as a chaperone protein in antigen presentation by HLA-DR to Th cells (Weenink & Gautam, 1997, Immunol Cell Biol 75:69-81). In addition, CD74 mediates macrophage migration inhibitory factor (MIF)-induced signal transduction as its cognate membrane receptor (Leng et al., 2003, J Exp Med 197:1467-1476), resulting in activation of ERK1/2 and protection from p53-dependent apoptosis in a process that requires CD44 as a signaling component and involves PKA and c-Src (Shi et al., 2006, Immunity 25:595-606). Moreover, the intracellular domain of CD74 (CD74-ICD) released from intramembrane proteolysis in the endocytic compartments can activate NF-κB to induce B-cell maturation (Becker-Herman et al., 2005, Mol Biol Cell 16:5061-5069), and this activity is further enhanced by stimulating CD74 with an agonistic antibody or MIF, either of which augments CD74-ICD release, increases Bcl-xL expression, and elevates the phosphorylation of both Akt and Syk (Starlets, 2006, Blood 107:4807-4816). In CLL cells (Binsky et al., 2007, Proc Natl Acad Sci USA 104:13408-13413) and B-lymphocytes (Gore et al., 2008, J Blot Chem 283:2784-2792), binding of CD74 to MIF initiates a signaling cascade that promotes cell survival via activation of NF-κB and secretion of IL-8, which can be inhibited by the antagonistic anti-CD74 MAb, milatuzumab (Binsky et al., 2007, Proc Natl Acad Sci USA 104:13408-13413; Shachar & Haran 2011, Leuk Lymphoma 52:1446-1454), thus further substantiating the rationale to develop milatuzumab-based therapeutic agents for the treatment of cancer (Stein et al., 2007, Clin Cancer Res 13:5556s-5563s) and autoimmune disease (Borghese & Clanchy, 2011, Exp Opin Ther Targets 15:237-251) by blocking the CD74/MIF pathway.

The commercial success of rituximab in treating certain B-cell malignancies and autoimmune disorders has stimulated much interest in developing new anti-CD20 antibodies with improved efficacy, as well as elucidating the in vitro and in vivo mechanisms of action for rituximab and its analogues (Lim et al., 2010, Haematologica 95:135-143). At present, next-generation anti-CD20 MAbs include human and humanized forms, with some claiming enhanced potency by antibody reengineering (Pawluczkowycz et al., 2009, J Immunol 183:749-758; Mossner et al., 2010, Blood 115:4393-4402). Although CD20 is a well-validated therapeutic target and despite more than 10 years of clinical use of rituximab and an expansive preclinical literature on the use of anti-CD20 MAbs in lymphoma models in vitro and in vivo, how rituximab or other anti-CD20 MAbs kill lymphoma cells is still being debated (Glennie et al., 2007, Mol Immunol 44:3823-3837) among the three principal mechanisms proposed, CDC, ADCC, and direct toxicity induced by signaling. Functional differences, as revealed by a variety of assays such as induction of homotypic adhesion, stimulation of calcium mobilization, association with lipid rafts, capability for effective CDC, ADCC or both, and requirement of hypercrosslinking for direct in vitro cytotoxicity, have been used (Cragg et al., 2003, Blood 101:1045-52) to classify anti-CD20 MAbs into type I, represented by rituximab, or type II, represented by tositumomab, with some data indicating that type II MAbs appear to outperform type I in preclinical models (Cardarelli et al., 2002, Cancer Immunol Immunother 51:15-24; Beers et al., 2008, Blood 112:4170-4177), which awaits confirmation in patients. We have previously noted (Rossi et al., 2008, Cancer Res 68:8384-8392) that one effective approach to converting a type I anti-CD20 MAb to a type II can be achieved by making the type I MAb multivalent, as shown by the HexAb generated from the type I veltuzumab, 20-(20)-(20), which exhibits biological properties attributable to both type II (for example, negative for CDC and calcium mobilization; positive for anti-proliferation, apoptosis, and homotypic adhesion) and type I (for example, positive for trafficking to lipid rafts).

The strategy to target both CD20 and CD74 with distinct MAbs was reported recently in a preclinical study using a combination of milatuzumab and rituximab plus a secondary crosslinking Ab in MCL lines and primary tumor cells (Alinari et al., 2011, Blood 117:4530-41), which showed that the treatment resulted in rapid cell death, generation of ROS, loss of mitochondria membrane potential, strong homotypic adhesion, and inhibition of p65 nuclear translocation. The observed cell death was attributed to a nonclassical apoptotic mechanism, because it lacks evidence of autophagy and caspase-activation, but requires the participation of actin and lysosomes. In the current study, we evaluated the potential of two anti-CD20/CD74 HexAbs for the therapy of MCL, and observed similar intracellular events to those obtained with milatuzumab and rituximab combined in the presence of a secondary crosslinking Ab, which include generation of ROS, loss of mitochondria membrane potential, inhibition of p65 nuclear translocation, absence of autophagy, and caspase-independence. Surprisingly, the two anti-CD

20/CD74 HexAbs, were capable of manifesting direct in vitro cytotoxicity in 3 MCL, 2 NHL, and 2 CLL cell lines, as well as inducing a significantly higher number of annexin V-positive cells in primary tumor samples from MCL and CLL patients, compared to untreated controls.

We also investigated the signaling pathways triggered in JeKo-1 cells by the two anti-CD20/CD74 HexAbs. Our findings suggest that the rapid and sustained activation of ERKs and JNK may contribute to cell death (Zhuang & Schnellmann, 2006, J Pharmacol Exp Ther 319:991-997), as shown in Raji and SU-DHL4 treated with tositumomab and radiation (Ivanov et al., 2008, Clin Cancer Res 14:4925-4934), in renal epithelial cells during oxidative injury (Di Mari et al., 1999, Am J Physiol 277:F195-F203), and in Raji and other B-lymphoma lines (including Jeko-1 and Granta-519) upon ligation to hL243 (anti-HLA-DR) MAb (Stein et al., 2010, Blood 115:5180-5190). We also found that both anti-CD20/CD74 HexAbs disrupt the NF-κB pathway by inhibiting the translocation of p65 from cytosol to the nucleus, and downregulate Bcl-xL, which may further promote cell death.

Ligation of various antibodies with receptors such as HLA-DR, CD 19, CD20, CD39, CD40, CD43, and others has been observed to induce homotypic adhesion in a panel of B-cell lymphoma lines (Kansas & Tedder, 1991, J Immunol 147:4094-4102). The anti-CD20/CD74 HexAbs, like the anti-CD20/CD22 HexAbs, also induced strong homotypic adhesion in JeKo-1 cells that was not observed with the parental MAbs alone or in combination. The HexAb-induced homotypic adhesion was blocked by inhibitors of actin polymerization, which also reduced cell death. Similar results have been observed with the combination of rituximab and milatuzumab in the presence of secondary crosslinking antibody in MCL lines (Alinari et al., 2011, Blood 117:4530-41). Our studies also indicate that the induction of homotypic adhesion by the anti-CD20/CD74 HexAbs is independent of their susceptibility to internalization, and neither classical apoptosis nor autophagy is involved in cell death, which appeared to closely resemble the actin- and lysosome-dependent cell death evoked by tositumomab and hL243 in Raji and SU-DHL4 (Ivanov et al., 2009, J Clin Invest 119:2143-2159).

When examined ex vivo using normal human blood spiked with JeKo-1 cells, 20-(74)-(74) depleted JeKo-1 cells more effectively than the parental MAb hA20, while hLL1 and 74-(20)-(20) did not show much activity. This improved efficacy may result from the fact that 20-(74)-(74), but not 74-(20)-(20), displays effector functions, as determined by ADCC and CDC. Both HexAbs, nevertheless, showed reduced but similar potency in depleting normal B cells, which could be due to a lower expression of CD20 or CD74 antigens, as well as the more mature cell type.

In summary, bispecific anti-CD20/CD74 HexAbs, as represented by 20-(74)-(74) and 74-(20)-(20), were successfully generated and their potential for therapy of MCL evaluated and demonstrated in preclinical studies. The key findings are as follows. (1) Effective inhibition of proliferation requires juxtaposing CD20 and CD74 in close proximity. (2) The observed direct in vitro cytotoxicity is accompanied by extensive homotypic adhesion, relocation of actin to the cell-cell junction, notable lysosomal enlargement, release of cathespin B into the cytosol, loss of mitochondria membrane potential, generation of ROS, deactivation of the PI3K/Akt signaling pathway, as well as rapid and sustained activation of ERK and JNK MAPKs. (3) Homotypic adhesion can be a first indicator for determining whether a certain antibody or combination of antibodies will display toxicity against antigen-expressing hematological cells. (4) Both 20-(74)-(74) and 74-(20)-(20) will be of use in additional B-cell malignancies, in particular CLL, and in autoimmune disease. These compounds constitute a new therapeutic class of anticancer antibodies.

### Example 3

### Anti-CD74 Antibodies Improve the Efficacy of Standard Treatments for B Cell Malignancies

Milatuzumab (humanized anti-CD74 monoclonal antibody) is in clinical evaluation for therapy of multiple myeloma, CLL and NHL (Berkova et al., 2010, Expert Opin Invest Drugs 19:141-49). CD74, the MHC class-II chaperone molecule, also functions as the cellular receptor for the proinflammatory cytokine, macrophage migration-inhibitory factor, and initiates a signaling cascade resulting in proliferation and survival (e.g., Leng et al., 2003, J Exp Med 197:1467-76). Preclinically, milatuzumab demonstrates therapeutic activity against various B-cell malignancies when used alone (Berkova et al., 2010, Expert Opin Invest Drugs 19:141-49), and the therapeutic efficacies of bortezomib, doxorubicin, and dexamethasone are enhanced in multiple myeloma cell lines when given combined with milatuzumab (Stein et al., 2009, Clin Cancer Res 15:2808-17). Milatuzumab acts through distinct mechanisms from rituximab, and exhibits different expression and sensitivity profiles. We examined the effects of milatuzumab given in combination with rituximab or fludarabine in human NHL, CLL, and ALL cell lines.

Methods

Three human NHL (WSU-FSCCL, Raji, and RL); two ALL (MN60 and REH), and two CLL (MEC-1 and WAC) cell lines were tested, with evaluation of therapeutic efficacies of milatuzumab and fludarabine performed in the NHL and CLL cell lines. The cell lines were selected to evaluate a range of different CD74 and CD20 expression levels (Table 16).

TABLE 16
Cell surface expression of CD74 and CD20 (mean fluorescence)

| | | Isotype control | CD74 | CD20 |
|---|---|---|---|---|
| | | (hMN14, | (hLL1, | (hA20, |
| | Cell line | anti-CEA) | milatuzumab) | veltuzumab) |
| NHL | Raji | 11.6 | 318.6 | 760.4 |
| | RL | 6.2 | 56.0 | 264.5 |
| | WSU-FSCCL | 5.3 | 20.9 | 36.6 |
| ALL | REH | 3.0 | 70.6 | 15.0 |
| | MN60 | 8.4 | 57.0 | 719.4 |
| CLL | MEC-1 | 5.4 | 41.4 | 270.5 |
| | WAC | 5.2 | 17.4 | 210.6 |

Results

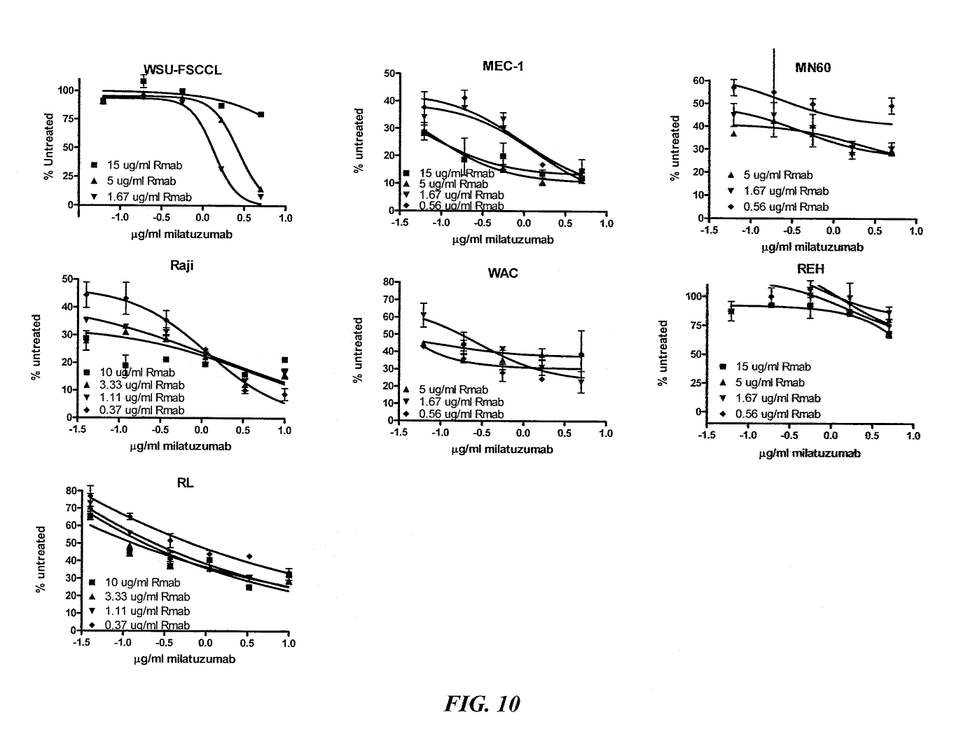

Anti-proliferative activity was augmented in vitro when milatuzumab and rituximab were combined (FIG. 10). For example in WSU-FSCCL cells, which are relatively insensitive to rituximab, inhibition of proliferation in the presence of 33.3 nM rituximab increased from 12.6±3.7% in the absence of milatuzumab to 85.5±0.0% (P=0.023) in the presence of 33.3 nM milatuzumab (FIG. 10). In Raji, a more sensitive cell line, inhibition of proliferation in the presence of 22.2 nM rituximab increased from 64.8±1.3% without milatuzumab to 86.6±0.9% (P=0.018) with 22.2 nM milatuzumab (FIG. 10). Significant increases in the anti-proliferative activity of rituximab were similarly observed in all but one of the tested NHL, CLL, and ALL cell lines (FIG. 10), with the exception of REH, which was not sensitive to killing by either milatuzumab or rituximab. Unlike rituximab, milatuzumab induced little or no ADCC or CDC (not shown). However, in vitro exposure of cells to milatuzumab did not affect rituximab mediated ADCC or CDC (not shown).

The effects of milatuzumab on mitochondrial membrane potential were examined using an alamar blue assay. Alamar blue (10%) was added to wells 24 h after each period of incubation and changes in alamar blue reduction were measured every fifteen minutes for a total of 4 hours. Rituximab-resistant cell lines were generated from the Raji parental cell lines by exposing cells to an escalating dose of rituximab (0.1 to 128 μg/ml), either without human serum (Raji 2R) or in the presence of human serum (Raji4RH). The combination of milatuzumab and rituximab was observed to result in a more potent decrease in the mitochondrial potential in rituximab-sensitive cell lines, but not in rituximab-resistant cell lines.

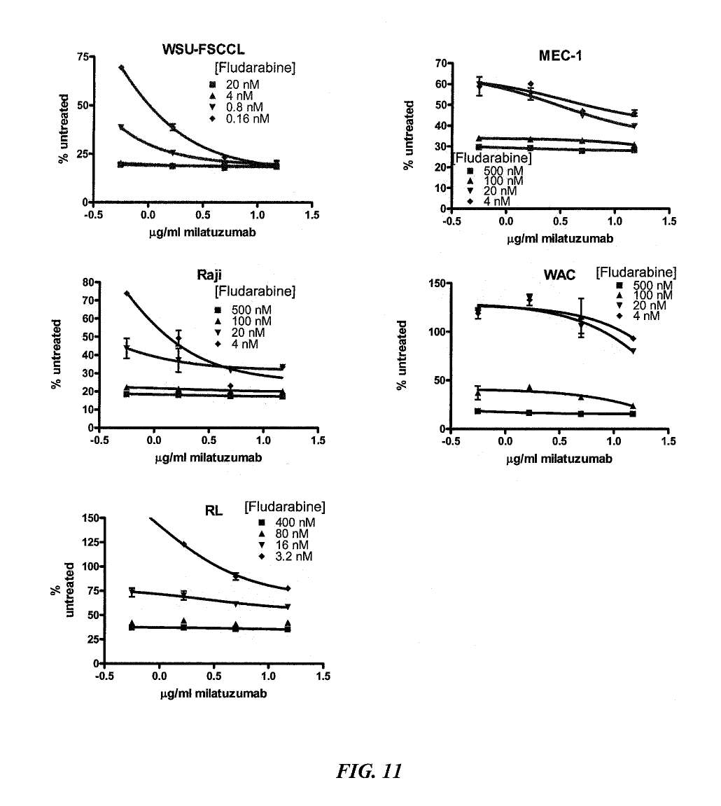

It was found that milatuzumab increased the efficacy of fludarabine-induced cytotoxicity in 3 NHL and 2 CLL cell lines (FIG. 11). For example, in Raji cells, which are relatively insensitive to fludarabine, inhibition of proliferation in the presence of 4 nM fludarabine increased from no inhibition in the absence of milatuzumab to 76.9±0.7% (P=0.009) in the presence of 33.3 nM milatuzumab (FIG. 11). In WSU-FSCCL cells, a more fludarabine-sensitive cell line, inhibition of proliferation in the presence of 0.8 nM fludarabine increased from 41.3±0.3% in the absence of milatuzumab to 79.7±0.1% (P<0.0001) with 33.3 nM milatuzumab (FIG. 11).

### CONCLUSIONS

Milatuzumab and other antagonistic anti-CD74 antibodies can significantly add to the efficacy of currently approved therapies, such as fludarabine and rituximab, for B cell diseases, including NHL, CLL and ALL.

What is claimed is:

1. A method of treating non-Hodgkin's lymphoma, comprising administering to a human patient with non-Hodgkin's lymphoma (NHL) a combination of an anti-CD74 antibody or antigen-binding fragment thereof and an anti-CD20 antibody or antigen-binding fragment thereof, wherein the patient is relapsed from or resistant to at least one therapy for NHL.

2. The method of claim 1, wherein the patient is relapsed from or resistant to treatment with rituximab.

3. The method of claim 2, wherein the combination of anti-CD74 and anti-CD20 antibody or fragment thereof is effective to treat a patient who is relapsed from or resistant to treatment with rituximab.

4. The method of claim 1, wherein the anti-CD20 antibody is veltuzumab.

5. The method of claim 1, wherein the anti-CD74 antibody is milatuzumab.

6. The method of claim 1, wherein the anti-CD20 antibody is administered at a dosage of 200 mg/m² once a week.

7. The method of claim 1, wherein the anti-CD74 antibody is administered at a dosage of 8, 16, and 20 mg/kg once a week or twice a week.

8. The method of claim 1, wherein the NHL is selected from the group consisting of follicular lymphoma, diffuse large B-cell lymphoma, mantle cell lymphoma, lymphoplasmacytic lymphoma, and marginal zone lymphoma.

9. The method of claim 1, wherein the NHL is indolent NHL.

10. The method of claim 1, wherein the incidence of Grade 3 or higher leukopenia, neutropenia, anemia, infusion reactions, hyperglycemia, fatigue and atrial tachycardia is 25% or less in the population of treated patients.

11. The method of claim 5, wherein the patient is administered dexamethasone prior to and after each dose of milatuzumab.

12. The method of claim 11, wherein dexamethasone is administered at a dose of 20 mg.

13. The method of claim 1, wherein the anti-CD20 antibody is administered on day 1 of weeks 1 to 4 and days 1 and 4 of weeks 12, 20, 28 and 36 of therapy,

14. The method of claim 1, wherein the anti-CD74 antibody is administered on day 2 of week 1 and days 1 and 4 of weeks 2 to 4.

15. The method of claim 1, wherein the anti-CD74 antibody is administered on days 1 and 4 of weeks 12, 20, 28 and 36.

16. The method of claim 1, wherein the anti-CD74 antibody is administered on day 2 of week 1 and day 4 of weeks, 2, 3, 4, 12, 20 and 36.

17. The method of claim 1, wherein the combination of anti-CD20 and anti-CD74 antibody does not induce a dose-limiting toxicity.

18. The method of claim 1, further comprising administering fingolimod to the patient.

19. The method of claim 1, further comprising administering acetaminophen, diphenhydramine, hydrocortisone and famotidine to the patient prior to antibody administration.

20. The method of claim 1, wherein combination therapy with anti-CD20 and anti-CD74 antibody is capable of inducing a complete response in the patient.

21. The method of claim 1, wherein combination therapy with anti-CD20 and anti-CD74 antibody results in an objective response rate of about 30% or higher.

22. A method of treating rituximab-resistant non-Hodgkin's lymphoma, comprising administering to a patient with rituximab-resistant non-Hodgkin's lymphoma a combination of veltuzumab (anti-CD20 antibody) and milatuzumab (anti-CD74 antibody).
